(19) United States
(12) Patent Application Publication
(10) Pub. No.: US 2018/0046754 A1
(43) Pub. Date: Feb. 15, 2018

(54) NORMALIZATION METHODS FOR MEASURING GENE COPY NUMBER AND EXPRESSION

(71) Applicant: AGENCY FOR SCIENCE, TECHNOLOGY AND RESEARCH (A*STAR), Singapore (SG)

(72) Inventors: Arsen BATAGOV, Singapore (SG); Surya Pavan YENAMANDRA, Singapore (SG); Vladimir KUZNETSOV, Singapore (SG)

(21) Appl. No.: 15/561,025

(22) PCT Filed: Mar. 24, 2016
(86) PCT No.: PCT/SG2016/050140

§ 371 Date: Sep. 22, 2017

(30) Foreign Application Priority Data
Mar. 24, 2015 (SG) 10201502276Q

(51) Int. Cl. G06F 19/22; C12Q 1/68; G06F 19/20

(52) U.S. Cl. CPC G06F 19/22; G06F 19/20; C12Q 1/6886; C12Q 2600/156

(57) ABSTRACT

The present invention provides method(s) for measuring gene copy number (CN) of a given locus of interest, comprising 1) obtaining the CN value of the locus of interest, 2) obtaining the CN value or values of one or more CN-invariant locus reference(s) (CNILR) in the biological sample, where the CNILR is a locus which is locally CN-invariant or a locus with a minimal coefficient of variation, 3) obtaining the CN value or values of one or more CN-invariant and survival insignificant locus reference reference(s) (CNISILR) determined based on survival prediction analysis for a specific subgroup; and 4) normalizing the CN value of the locus of interest by the CN values of one or more CNISILRs if defined, otherwise normalizing the CN value of the locus of interest by the CN values of said one or more CNILRs. In one embodiment, the CNILRs or CNISILRs is one or more loci from the group consisting of XRCC5, AUTS2, EIF5, PARN, YEATS2 and FHL2. Also encompassed are kits and computer program or computer device for use in the methods of the invention.

### FIELD OF THE INVENTION

The present invention relates to method(s) for measuring gene copy number and gene expression, quantitative PCR, qRT-PCR, normal individuals, medical conditions including the patients with cancer, ovarian cancer, ovarian serous adenocarcinoma, cancer diagnosis, cancer detection, therapy monitoring and laboratory diagnostics.

### BACKGROUND OF THE INVENTION

The gene copy number (also gene “copy number variants” or CNV) is the number of copies of a particular gene in the genotype of an individual. In the human genome, DNA encodes more than 25,000 protein coding genes and many thousands of non-protein coding genes. It was generally thought that genes in somatic cells were almost always present in two copies in a genome. However, recent discoveries have revealed that larger numbers of the segments of DNA could be observed. The size of such segments ranges from hundreds to millions of DNA bases, providing variation in DNA segment/gene copy-number. Such differences in the CNV of the individual genomes occurs in normal body cells, contributing to the organism's uniqueness. However, these DNA amount changes also influence most traits including susceptibility to disease. CNV can encompass individual genes and their clusters leading to dosage imbalances. For example, genes that were thought to always occur in two copies per genome have now been found to sometimes be present in one, three, or more than three copies. In various medical conditions and disease progression states, some DNA loci containing key regulatory genes are missing.

Gene or DNA copy number (CN) is usually measured by an average number of DNA copies per genome per cell in a biological sample. Gene copy number variation (CNV) is observed in normal tissue samples and is amplified in certain diseases, such as cancers. It has previously been demonstrated that CNV of a given gene directly affects its expression. The exact relationship between the CNV and the gene expression values is poorly studied but it is thought to be a nonlinear relationship which depends on cell, tissue, organism and medical conditions. The accurate and reproducible detection of CN and CNV of a given genome locus (or loci) and an establishment of their quantitative interconnection with the variation of expression of a gene belonging to a given CNV locus (or loci) is a great challenge. A practical solution of this problem is urgently needed for optimization of healthcare strategies, evaluation of the status of normal individuals and for diagnosis, prognosis and prediction for patients with medical conditions.

qPCR-based assays are considered as “gold standards” for detecting a variety of medical conditions attributed to gene expression changes and are broadly used in common clinical practice. Gene expression level in the cells and/or tissue samples is usually ranged within 5-6 orders of magnitude and a detection of the variation of such characteristics is provided by qPCR-based techniques, often with high accuracy. However qPCR-based assay interpretation is majorly dependent on measurement of cycle threshold (CT) values of the target gene(s) relative to CT values of reference/normalizing gene(s) (e.g. ACT B, GAPDH etc.). This condition might be a limitation in the context of cell or tissue specification and of bio-medical or environmental conditions, due to a systematic or random error variation that could occur in the reference/normalizing gene(s). In particular, some of the reference/normalizing gene(s) can also vary in a correlated manner with expression levels of the gene(s) of interest in a given cell/tissue sample. For example, GAPDH, commonly used as a reference gene, is considered to be an oncogene in breast cancer as its expression level is highly correlated with cancer progression level. Therefore, this gene cannot be used as an invariant reference for breast cancer assays. The variation in expression levels of the reference/normalizing gene(s) could also be prone to non-specific and poorly controlled noise, due to the heterogeneous sample cell composition. Thus, in many cases conventional reference/normalizing gene(s) might not be usable as “universal” and “independent” controls providing robust, unbiased and accurate measurements of the expression of a given gene of interest estimated via CT value analysis calculations for a qPCR assay. An identification of adequate reference/normalizing gene(s) for the accurate, robust and reliable detection of the DNA copy number variation (CNV) of a given gene locus using qPCR-based assays appears to be more challenging. Firstly, the dynamical range of CNV detection is limited to a few delta-delta CT-values, which is a less accurate and more noise-prone measurement procedure than that of gene expression. Secondly, the actual measurement in a cell/tissue sample is defined by delta-delta CT-values, averaged across many cells of a biological sample. CNV of the “control” genes across a single sample can be observed even in normal tissue samples, and is much more amplified in some pathological cases. Thirdly, in certain diseases, such as serous ovarian carcinoma, CNV of a given gene might directly affect the gene expression. The exact relationship between the CNV and the expression values is poorly understood and might be non-linear. Present methods for measuring gene CN and expression have been designed ignoring these facts. Therefore, gene CN and expression values obtained with any existing measurement method are affected by the unobserved CNV. Therefore, in such cases the CNV of the reference gene set also affects the observed expression values of any other gene measured in a given assay. Thus, the problem of indefinite CNV may invalidate any gene expression measurement. In many situations, such as those indicated above, more accurate, unbiased and robust reference/normalizing gene(s) should be identified, and appropriate primers should be optimized for use in detecting gene expression (mRNA/ncRNA) and CN (DNA) level.

### SUMMARY OF THE INVENTION

Some embodiments relate to a method for determining a quantitative measure of a target gene in a biological sample from a subject, the method comprising:
conducting an assay to measure respective quantities of the target gene and one or more reference genes or loci; and
normalizing the quantity of the target gene using the quantity or quantities of the one or more reference genes or loci, or a normalization function thereof;
wherein the one or more reference genes or loci are copy number-invariant genes or loci.

Other embodiments relate to a kit for obtaining reference gene measurements in one or more biological samples, the kit comprising oligonucleotide primers capable of binding to and/or amplifying at least a portion of the nucleic acid sequence, and/or cDNA derived therefrom, of at least one gene selected from the group consisting of: XRCC5; AUTS2; EIF5; PARN; YEATS2; and FHL2.

According to a preferred embodiment of the kit, the primer sequences are selected from or derived from oligonucleotide sequences identified in Table 6 as SEQ ID Nos: 1-24.

According to a preferred embodiment of the kit, the primers are capable of binding to and/or amplifying at least a portion of the nucleic acid sequence, and/or cDNA derived therefrom, of at least one locus selected from Table 1, Table 2, Table 3, Table 4, Table 5, Table 8, Table 9, Table 10, Table 11, Table 13 or Table 14.

Further embodiments relate to a computer program or a computer device comprising a computer program which is capable of implementing the method according to any aspect of the present invention.

Further embodiments relate to a computer-implemented method for identifying reference genes and/or loci for relative quantitation of a target gene or locus, the method comprising:
receiving, by a reference gene/locus identification component, training data indicative of: copy numbers of a plurality of genomic segments in a plurality of pathological and/or non-pathological biological samples; corresponding RNA expression levels of genes/loci within or overlapping with said segments; and ranges of genomic coordinates of said segments;
assigning respective ones of the plurality of genomic segments to one of a plurality of non-overlapping genomic partitions;
determining, by the reference gene/locus identification component from the copy numbers of genomic segments in respective partitions, invariant partitions which are not subject to copy number variation; and
identifying, by the reference gene/locus identification component using RNA expression levels of genes/loci in the invariant partitions, a set of reference genes/loci comprising genes/loci which do not substantially vary in expression level across the plurality of biological samples.

Yet further embodiments relate to a computer-implemented method for identifying reference genes/loci for relative quantitation of a target gene/locus, the method comprising:
receiving, by a reference gene/locus identification component, training data indicative of: copy numbers of a plurality of genomic segments in a plurality of pathological and/or non-pathological biological samples and ranges of genomic coordinates of said segments;
assigning respective ones of the plurality of genomic segments to one of a plurality of non-overlapping genomic partitions;
determining, by the reference gene/locus identification component from the copy numbers of genomic segments in respective partitions, invariant partitions which are not subject to copy number variation.

Yet further embodiments relate to a method for measuring target gene(s) DNA copy number in one or more samples, the method comprising:
identifying one or more reference loci by a method according to any of the above embodiments;
for each sample, obtaining copy number measurements for the one or more reference loci;
for each reference locus, obtaining a numeric integrative measure of its DNA copy number values across the training data samples as a normalization factor;
for each of the one or more samples, obtaining the copy number value of the target locus (or loci); and
for each DNA copy number value of the target locus (or loci), obtaining its normalized copy number value by applying a normalization procedure using the normalization factor and a normalization function.

Further embodiments relate to a system for identifying reference genes and/or loci for relative quantitation of a target gene or locus, the system comprising:
a reference gene/locus identification component which is configured to:
receive training data indicative of: copy numbers of a plurality of genomic segments in a plurality of pathological and/or non-pathological biological samples; corresponding RNA expression levels of genes/loci within or overlapping with said segments; and ranges of genomic coordinates of said segments;
assign respective ones of the plurality of genomic segments to one of a plurality of non-overlapping genomic partitions;
determine, from the copy numbers of genomic segments in respective partitions, invariant partitions which are not subject to copy number variation; and
identify, using RNA expression levels of genes/loci in the invariant partitions, a set of reference genes/loci comprising genes/loci which do not substantially vary in expression level across the plurality of biological samples.

Yet further embodiments relate to a system for identifying reference genes/loci for relative quantitation of a target gene/locus, the system comprising:
a reference gene/locus identification component which is configured to:
receive training data indicative of: copy numbers of a plurality of genomic segments in a plurality of pathological and/or non-pathological biological samples and ranges of genomic coordinates of said segments;
assign respective ones of the plurality of genomic segments to one of a plurality of non-overlapping genomic partitions;
determine, from the copy numbers of genomic segments in respective partitions, invariant partitions which are not subject to copy number variation.

Other embodiments relate to a non-transitory computer readable medium having program instructions stored thereon for causing at least one processor to carry out the method according to any of the above embodiments.

Embodiments of the present disclosure relate to a novel method for obtaining accurate CN and gene expression measures of a given gene of a given subject via normalizing the measured values onto CN of the proposed DNA sequences (rtPCR/qPCR) primers associated with one (or more) of the obtained reference genes selected by a reference gene identification method which works at the genome level across populations of individuals and diverse medical conditions.

In certain embodiments, specified DNA sequences of a reference gene set, along with loci coordinates of the respective primers, might be optimized for a given patho-biological context and medical conditions. The practical efficacy/power of embodiments of the method is demonstrated using epithelial ovarian cancer (EOC) samples. Embodiments propose a reference gene set previously never used as a reference or normalization control in qPCR-based assays. This set is proposed for use in detection of expression and DNA copy number variation in ovarian serous adenocarcinoma samples. Embodiments also provide a computational method allowing one to select “reference and normalization” genes for any sample set, sharing specific biological or pathological characteristics, such as tissue of origin or/and medical condition.

Some embodiments relate to an in vitro method for obtaining information on the number of DNA copies (CN) of a given locus of interest in a biological sample, the method comprising:

i) obtaining the CN value of the locus of interest in the biological sample;

ii) obtaining the CN value or values of one or more CN-invariant locus reference(s) (CNILR) in the biological sample, wherein the CNILR is defined as a which is locally CN-invariant, or as a locus with a minimal coefficient of variation value of its CN values across said group;

iii) obtaining the CN value or values of or one or more CN-invariant survival-insignificant locus reference(s) (CNISILR), wherein the CNISILR being defined as a CNILR, whose CN value, or any expression value of the genes within the locus, cannot define more than one subgroup of said group, based on survival prediction analysis; and

iv) normalizing the CN value of the locus of interest by the CN value of said one or more CNISILRs if defined, otherwise normalizing the CN value of the locus of interest by the CN value of said one or more CNILRs.

In a preferred embodiment, said one or more CNILRs in the biological sample is/are determined by:

i) providing a representative reference data set containing measurements of genome-wide CN variation with respect to a group of samples;

ii) identifying a set of loci with the lowest variation across the reference data set as the reference loci;

iii) ranking the reference loci by their median CN values across the reference data set; and

iv) selecting one locus or a set of loci with the highest median CN value(s) as the CNILR(s).

In another preferred embodiment, said one or more CNISILRs in the biological sample is/are determined by:

i) providing a representative reference data set containing measurements of genome-wide CN variation with respect to a group of samples;

ii) identifying a set of loci with the lowest variation across the reference data set as the reference loci;

iii) identifying a subset of loci, whose functions and/or transcriptional activity are not statistically associated in the reference data set, as loci with no significant statistical association;

iv) ranking the loci with no significant statistical association by the coefficients of variation of the expression values of the transcripts originating in these loci across the reference data set; and

v) selecting one locus or a set of loci with the lowest coefficient(s) of variation of the CN values as the CNISILRs.

The normalization may be conducted by normalizing the CN value of the locus of interest by the CN value of the CNISILRs. Alternatively, or in addition, normalization is conducted by normalizing the CN values of the locus of interest by the median CN values of more than one CNISILRs. Normalization may also be conducted by normalizing the CN value of the locus of interest by the CN value of one CNILR or by the median CNNILRs.

According to a preferred embodiment, said one or more CNILRs or CNISILRs is one or more loci from the group consisting of: XRCC5; AUTS2; EIF5; PARN; YEATS2; and FHL2.

More particularly, said one or more CNILRs or CNISILRs is/are selected from the loci identified in Table 1, Table 2, Table 3, Table 4, Table 5, Table 8, Table 9, Table 10, Table 11, Table 13 or Table 14.

According to a preferred embodiment, said one or more CNILRs or CNISILRs is/are selected if the coefficient of variation is less than a computationally or empirically predetermined threshold is equal to 0.05.

Some embodiments relate to an in vitro method for determining the CN of a target gene in a biological sample, the method comprising:
1. obtaining the CN measurement of one or more CN-invariant genes
2. obtaining the CN measurement of the target gene
3. determining the CN value of the target gene from the ratio of the first two measurements.

Other embodiments relate to a method for determining the set of CN-invariant loci in a given set of samples, the method comprising:
1. obtaining the set of samples as the training set
2. for the samples in the training set, obtaining the genome-wide segmentation by uniform CN values
3. for each said CN segment determining its CN value in each sample
4. from the CN of the segments across all the samples, calculating the upper and lower CN thresholds that would mark a segment as amplified or deleted if its CN is above the upper or below the lower threshold, respectively
5. using the upper and the lower CN thresholds, identify the CN-aberrated (i.e. amplified or deleted) segments across all the samples
6. partitioning the genome in non-overlapping intervals without gaps (e.g. cytobands)
7. define individual loci in the genomic coordinates (e.g. genomic coordinates of genes)
8. for each genomic partition and each locus, identifying the number CN-aberrated segments overlapping with its genomic coordinates
9. identifying the partitions and the loci containing no CN-aberrated segments as CN-free loci and partitions, respectively
10. identifying such said CN-free loci that are located within the genomic coordinates of the said CN-free partitions as CN-invariant loci.

Further embodiments relate to an in vitro method for determining the expression of a target gene in a biological sample, the method comprising:
1. obtaining the gene expression measurement of one or more CN- and expression-invariant genes
2. obtaining the gene expression measurement of the target gene
3. determining the gene expression value of the target gene from the ratio of the first two measurements.

The CN value of the locus of interest and/or of said reference locus or loci in the biological sample may be determined as a gene expression value originating from a transcript of said locus.

In a preferred embodiment of any aspect of the present invention, the sample is obtained from cells or tissues from cancer patients or cell cultures derived from cancer patients.

The cancer patients may have a cancer type or subtype selected from ovarian cancer, breast invasive carcinomas, head and neck squamous cell carcinoma, lung adenocarcinoma, lung squamous cell carcinoma, prostate adenocarcinoma, colon adenocarcinoma, stomach adenocarcinoma, hepatocellular carcinoma, or cervical squamous cell carcinoma.

In a preferred embodiment, the sample is obtained from cells or tissues obtained from myocardial infarction patients or cell cultures derived from myocardial infarction patients.

Yet further embodiments relate to a method for determining the set of CN- and expression-invariant loci that can be used as a references for target gene expression measurements, the method comprising:
1. obtaining the set of CN-invariant loci for a given training set of samples
2. for each said CN-invariant locus, measuring across the samples the expression of the gene (or genes) located within the genomic coordinates of the locus
3. for each said CN-invariant locus, identifying a single gene with the highest measure of variation (e.g. coefficient of variation) of expression across the samples as the representative gene
4. from the list of the loci—representative gene pairs selecting such whose measure of variation is smaller than a given threshold (e.g. coefficient of variation less than 0.05) as the set of CN-invariant loci that can be used as references for target gene expression measurements

Yet further embodiments relate to a method for determining the optimal range of gene expression values that can be measured using the CN- and expression-invariant genes as references.

Yet further embodiments relate to CN- and gene expression measurements in ovarian cancer samples.

The present invention is further defined in accordance with the claims appended hereto.

### DETAILED DESCRIPTION

The present invention will now be further described by way of example and with reference to the Figures which show:

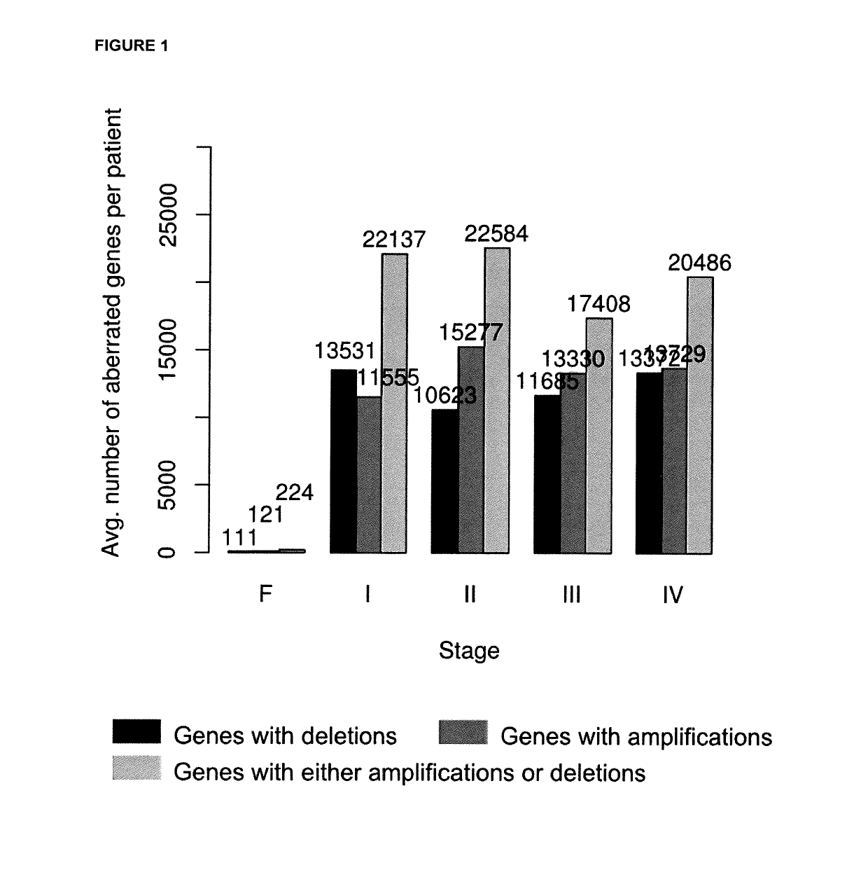

FIG. 1. The majority of genes in HG-SOC samples obtained from patients at any stage of the disease contain CNVs. The disease stages are denoted with Roman numerals (I-IV). Fallopian tube samples (denoted as “F”) obtained from HG-SOC-affected patients were used as a control;

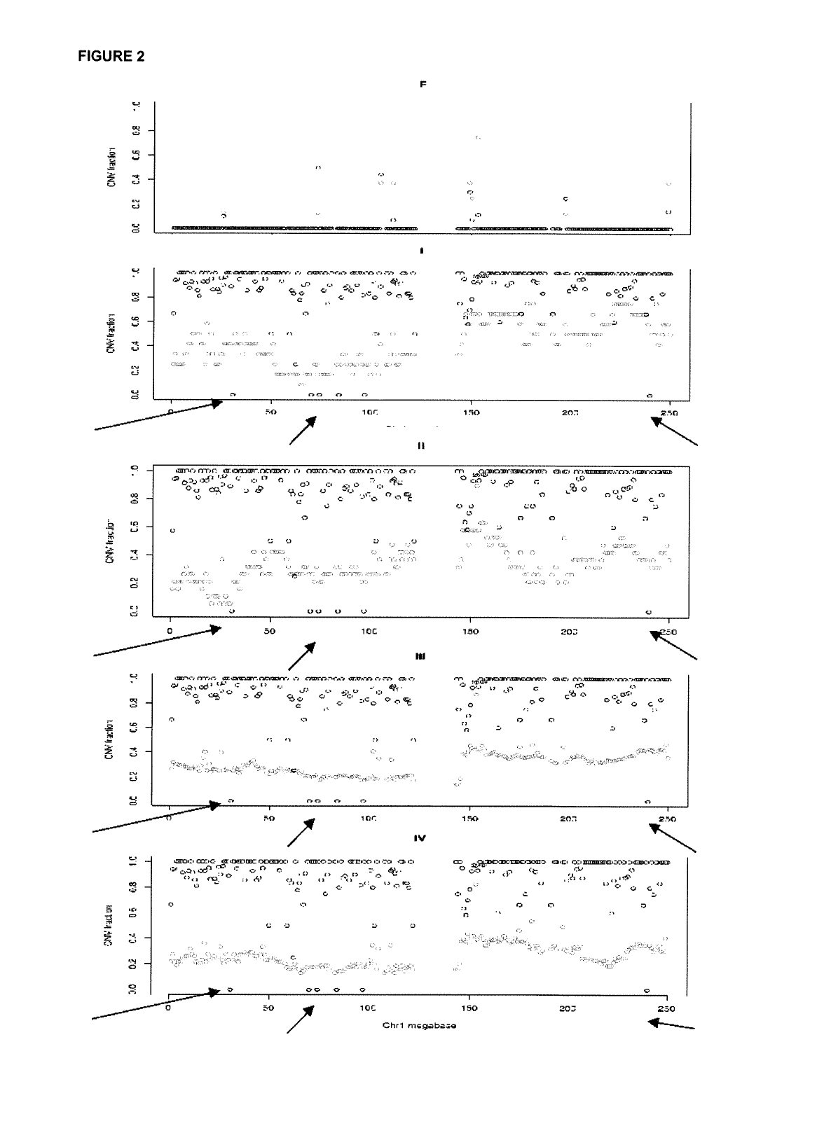

FIG. 2. CNV in chromosome 1 of HG-SOC samples (stages I-IV) and fallopian tubes (“F”) per megabase of the genomic distance (X axis). The Y axis shows the fraction of a) samples with CNV in a given megabase (black circles) and b) genes with CNV in a given megabase (grey circles). The arrows indicate the CNV-invariant regions that are used as sources of CNV-invariant genes;

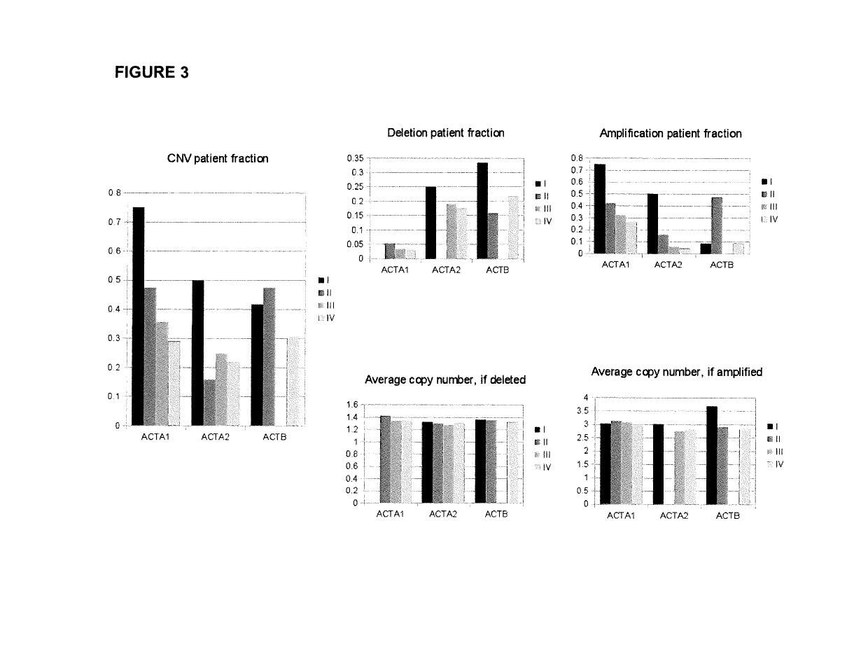

FIG. 3. Actin family genes reveal CNV in HG-SOC patients;

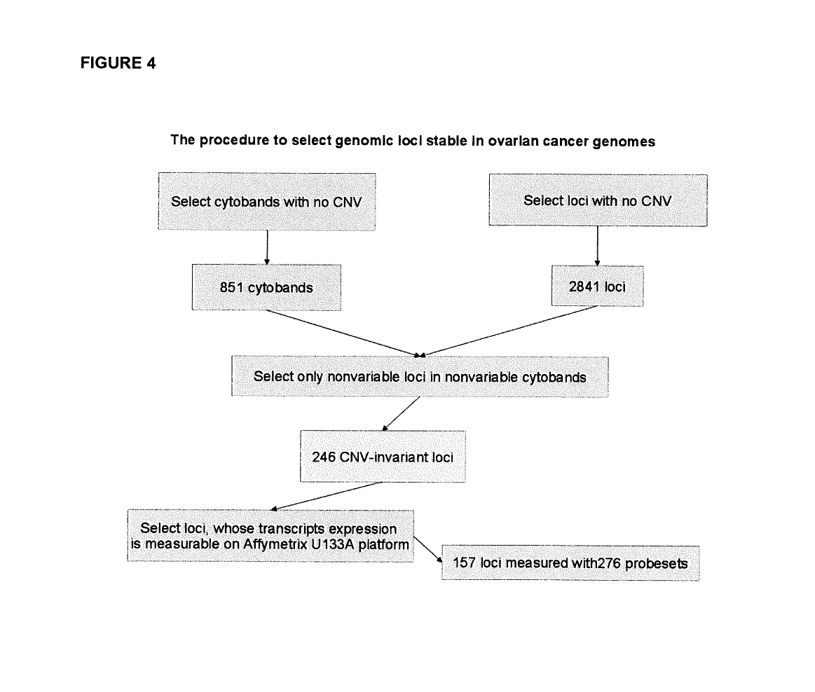

FIG. 4. An embodiment of an algorithm to choose CNV-invariant genes;

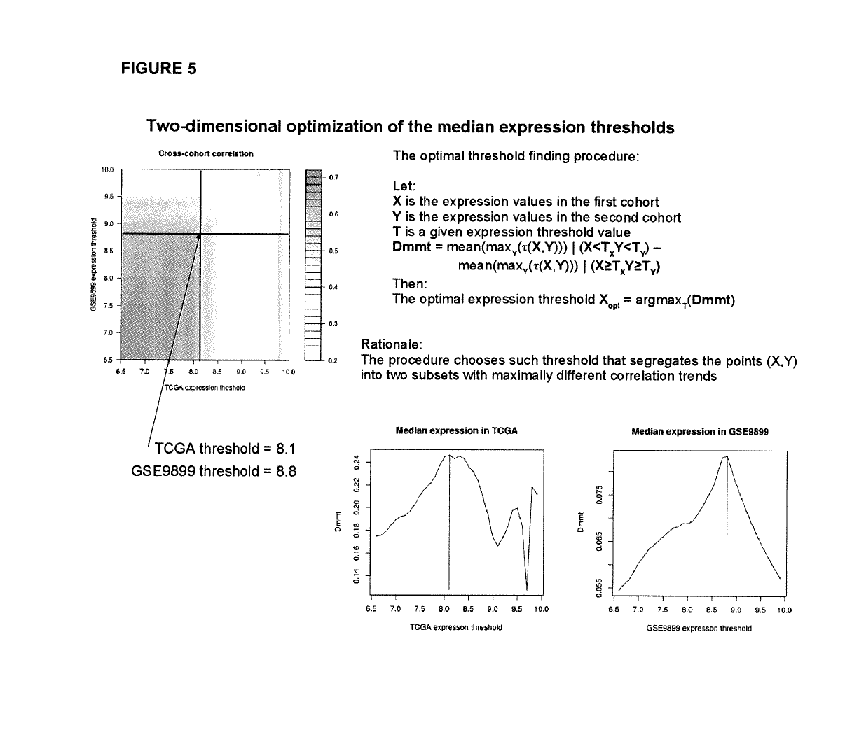

FIG. 5. An embodiment of an algorithm to choose the gene expression range optimal for using the CNV-invariant genes as references for gene expression measurements;

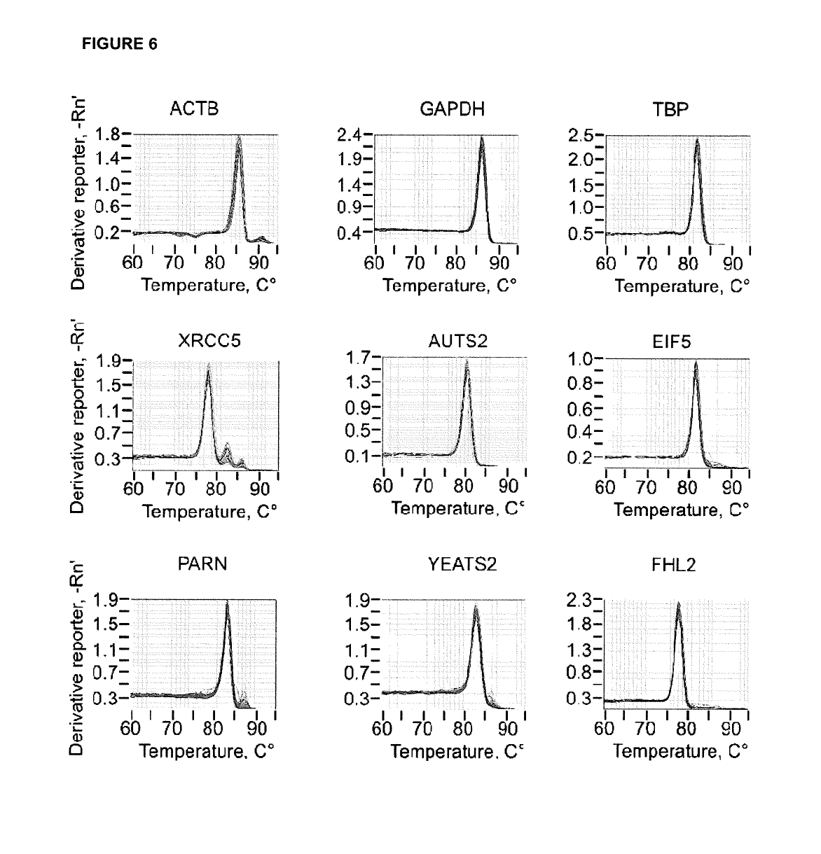

FIG. 6. Primer melting curves for exemplary reference genes;

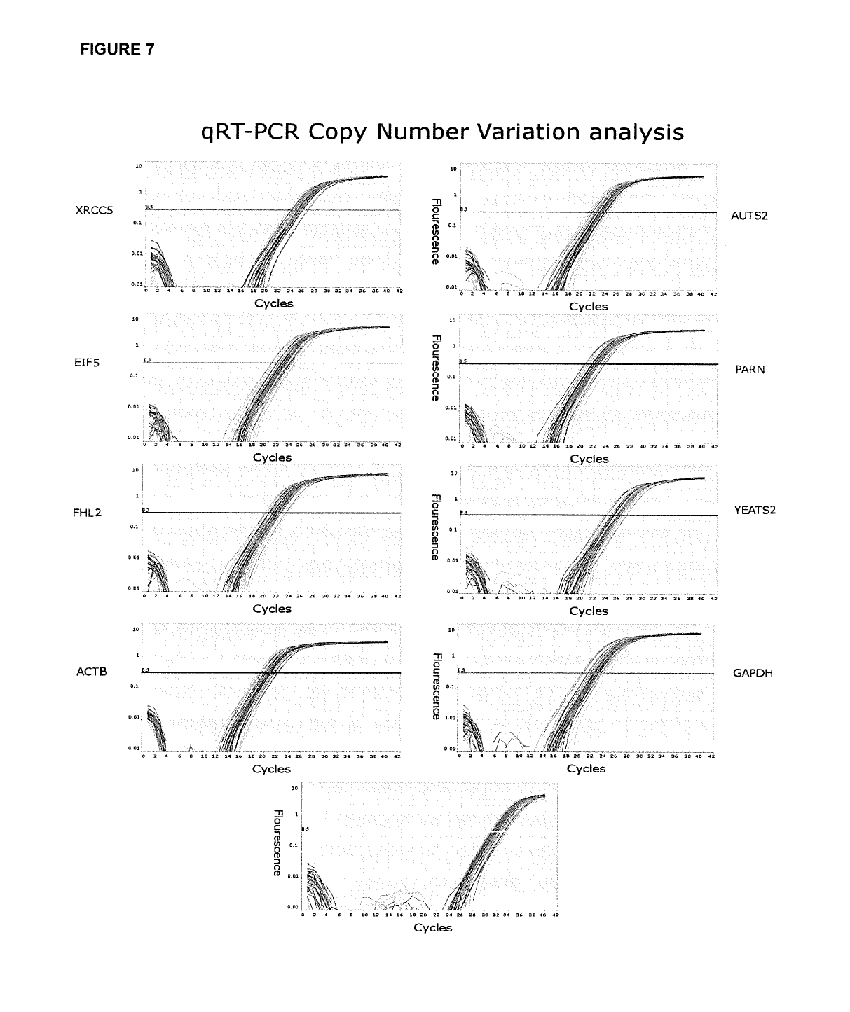

FIG. 7. Reproducibility of the qPCR signal measuring the reference genes CN values in biological replicas;

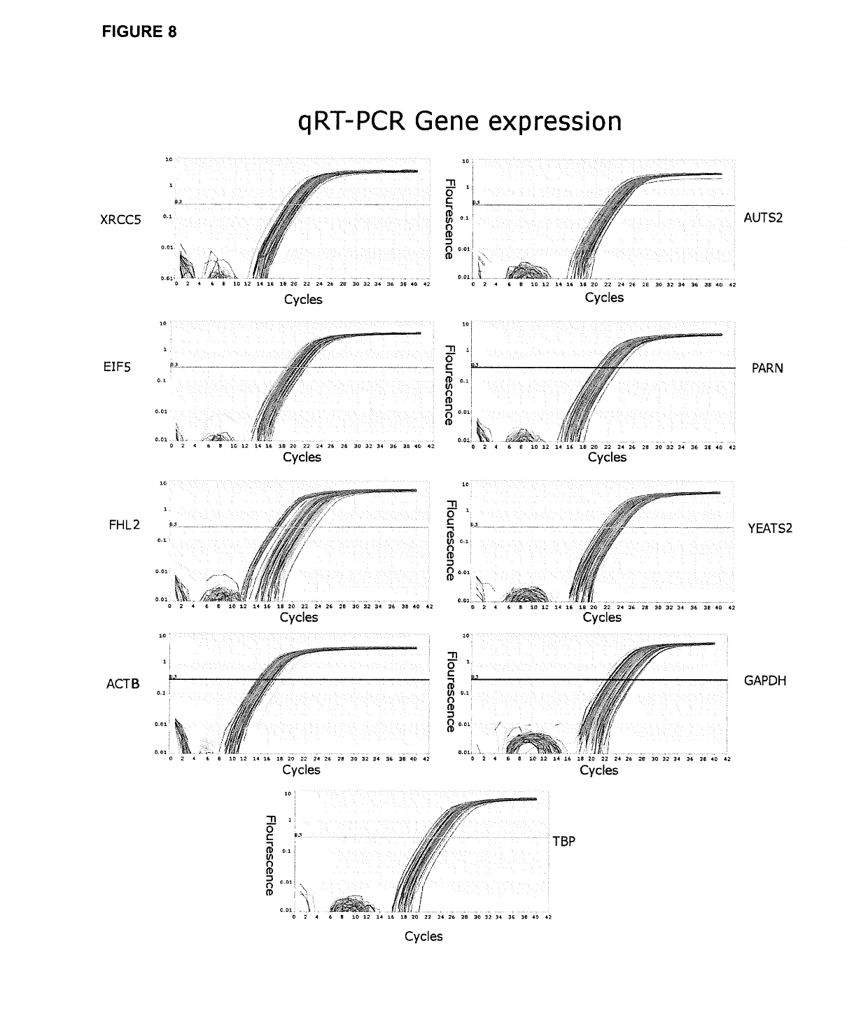

FIG. 8. Reproducibility of the qPCR signal measuring the reference genes expression values across biological replicas;

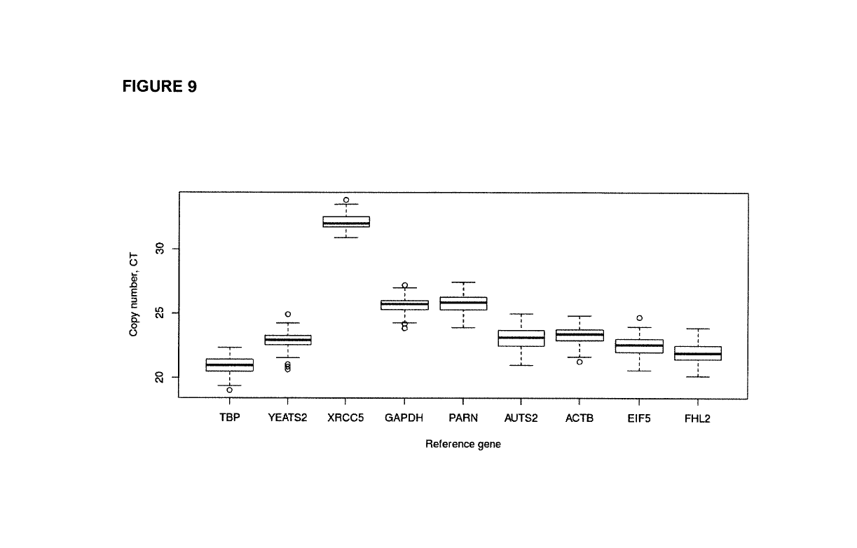

FIG. 9. The CT values variation obtained from the qPCR of the reference genes genomic DNA;

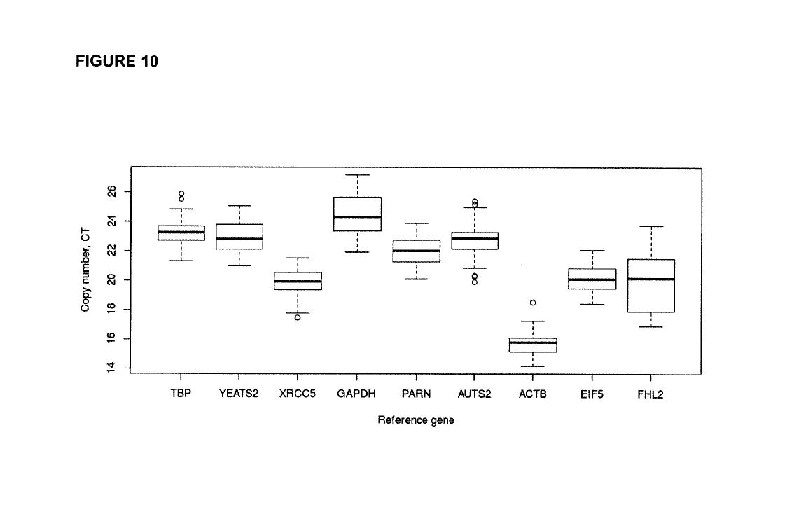

FIG. 10. The CT values variation obtained from the qPCR of the reference genes expression;

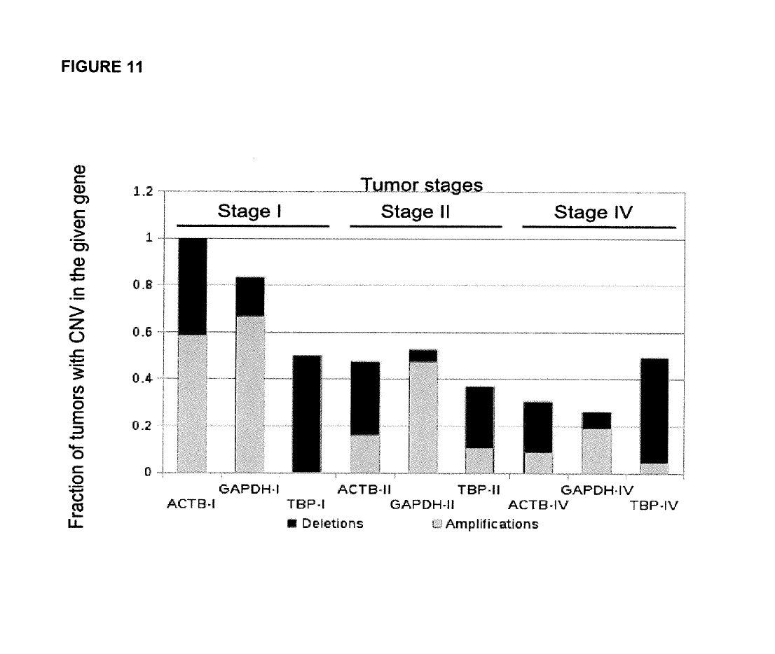

FIG. 11. The copy number variation, detected with CGH microarrays, within the genes most commonly used as references for qRT-PCR measurements;

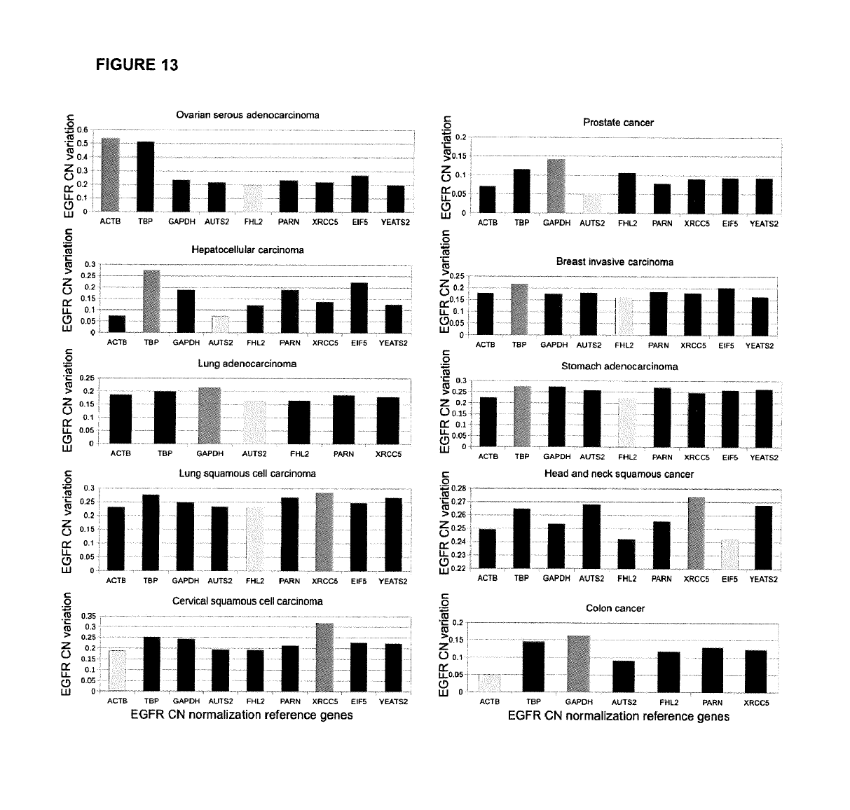

FIG. 12. The qPCR measurements of MECOM DNA copy number across ovarian serous adenocarcinoma tumor (T) and normal ovarian epithelium (N) control samples. The expected MECOM CN was obtained by normalization of its CT values by the median values of one of the normalziation reference genes. ACTB was selected as the traditional normalization reference. AUTS2, YEATS2, EIF5, XRCC5, and PARN were selected to represent the normalization references obtained by the proposed method. A) the difference between the tumor and the control median MECOM CN (the Wilcoxon test P-values are given); B-C) coefficient of variation of the MECOM CN across the tumor (B) and the control (C) samples; D-G) the estimated MECOM CN in the individual tumor (T) and control (N) samples;

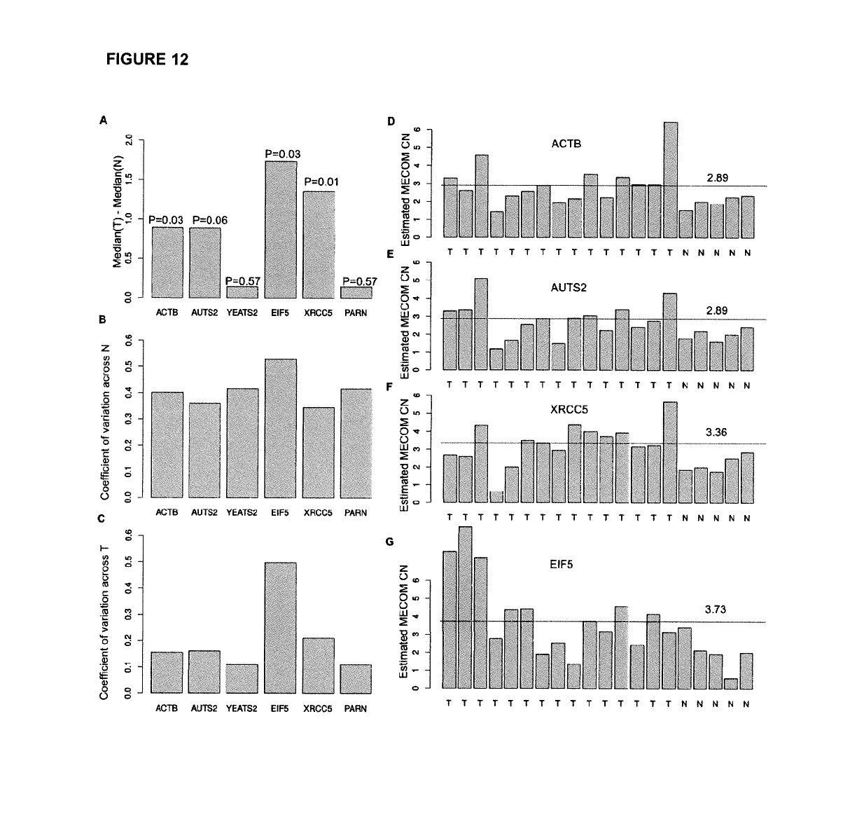

FIG. 13. Application of the present candidate loci, instead of traditional control loci (ACTB, TBP, and GAPDH), can improve an existing DNA-based clinical diagnostic assay Therascreen EGFR EGQ PCR Kit (Qiagen) measuring the DNA copy number of EGFR gene. Genes from our panel designed specifically for ovarian cancer, can improve the coefficient of variation of the EGFR DNA copy number in 8 out of 10 most common cancers, covering 50% of all cancer patients. Two reference loci providing the lowest and the highest variation of the EGFR CN measurements across the given samples are marked with the dark grey and the light grey colours, respectively;

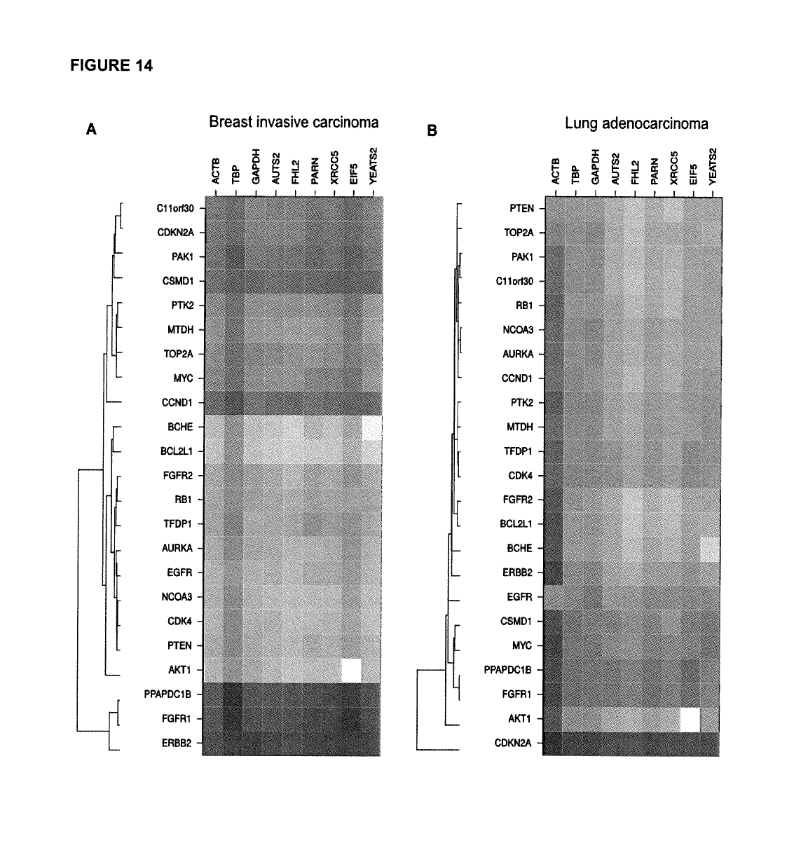

FIG. 14. Application of the candidate reference loci can improve an existing DNA-based assay Human Breast Cancer Copy Number PCR Array (Qiagen) measuring the DNA copy number of 23 loci reported to vary in breast cancer tumors. Across the breast invasive carcinoma (A), for 22 out of the 23 loci the lowest variation is obtained with the proposed candidate reference loci used as normalization controls, but not with the traditional control loci (ACTB, TBP, and GAPDH). Across the lung adenocarcinoma samples (B), for all 23 indicator loci of the assay the median variation of the markers obtained with our control loci was lower than the lowest variation obtained using any of the traditional control loci. Each cell of the matrix displayed as a rectangular heat map (in each panel) represents expression a gene of interest (in rows) normalized by a given reference locus (in columns). The colour intensity in each cell represents the expression value (growing from white to black);

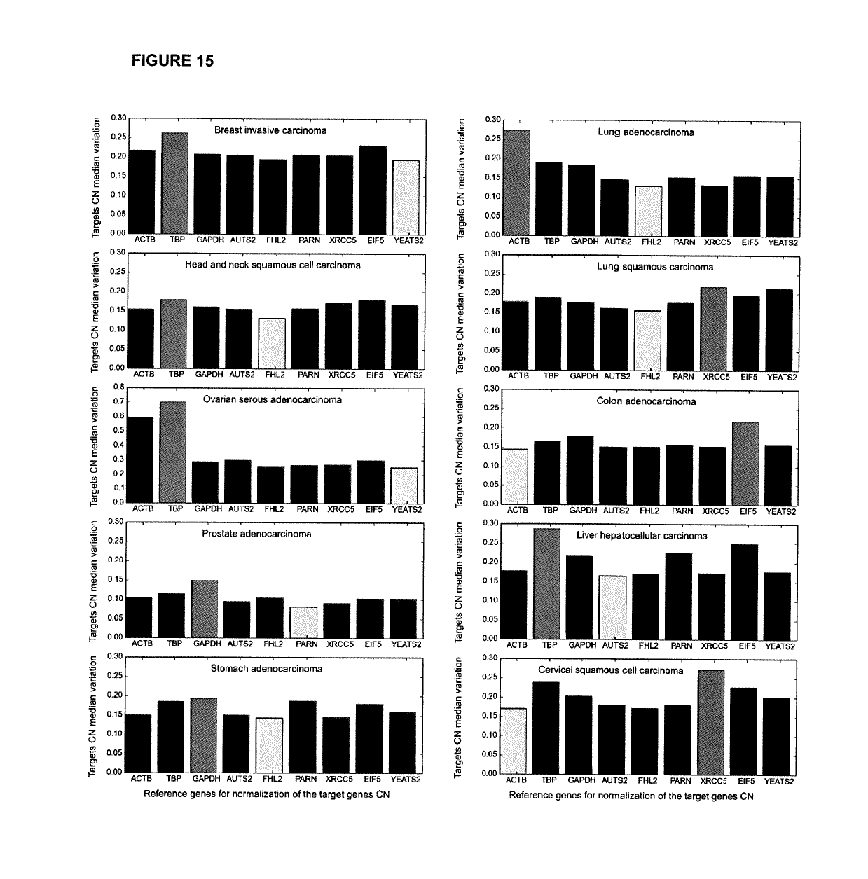

FIG. 15. Application of the present candidate loci can improve an existing DNA-based assay Human Breast Cancer Copy Number PCR Array (Qiagen) measuring the DNA copy number of 23 loci reported to vary in the breast cancer tumors. Two reference loci providing the lowest and the highest variation of the median CN measurements across the given 23 loci of interest, are marked with the dark grey and the light grey colours, respectively;

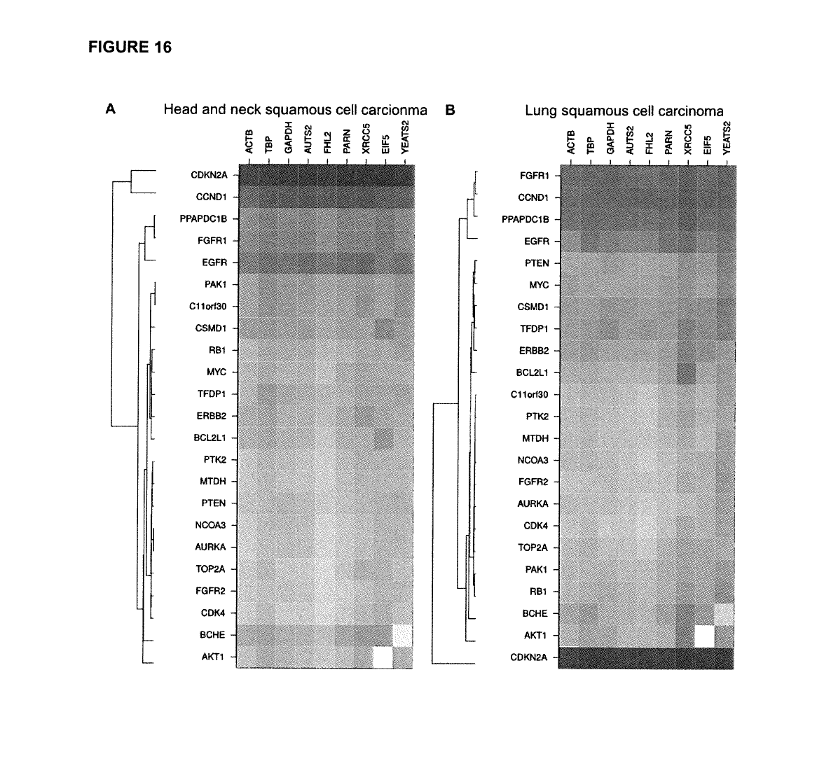

FIG. 16. Application of the present candidate loci can improve the Human Breast Cancer Copy Number PCR Array (Qiagen) applied to analysis of head and neck squamous cell carcinoma (A) and lung squamous cell carcinoma (B). Each cell of the matrix displayed as a rectangular heat map (in each panel) represents expression a gene of interest (in rows) normalized by a given reference locus (in columns). The colour intensity in each cell represents the expression value (growing from white to black);

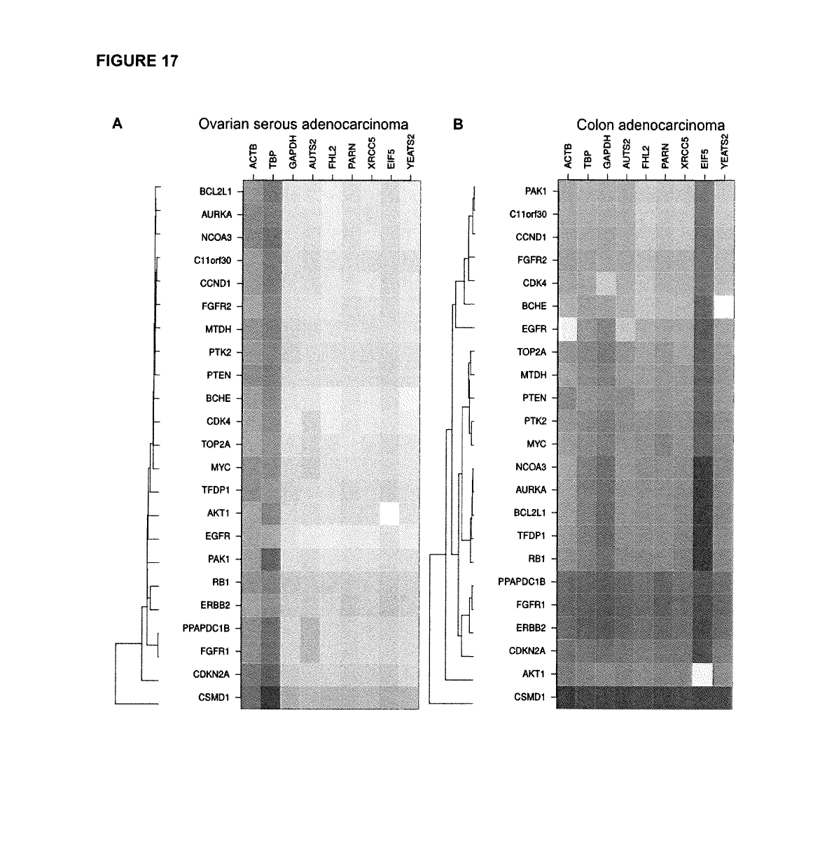

FIG. 17. Application of the present candidate loci can improve the Human Breast Cancer Copy Number PCR Array (Qiagen) applied to analysis of ovarian serous adenocarcinoma (A) and colon adenocarcinoma (B) Each cell of the matrix displayed as a rectangular heat map (in each panel) represents expression a gene of interest (in rows) normalized by a given reference locus (in columns). The colour intensity in each cell represents the expression value (growing from white to black);

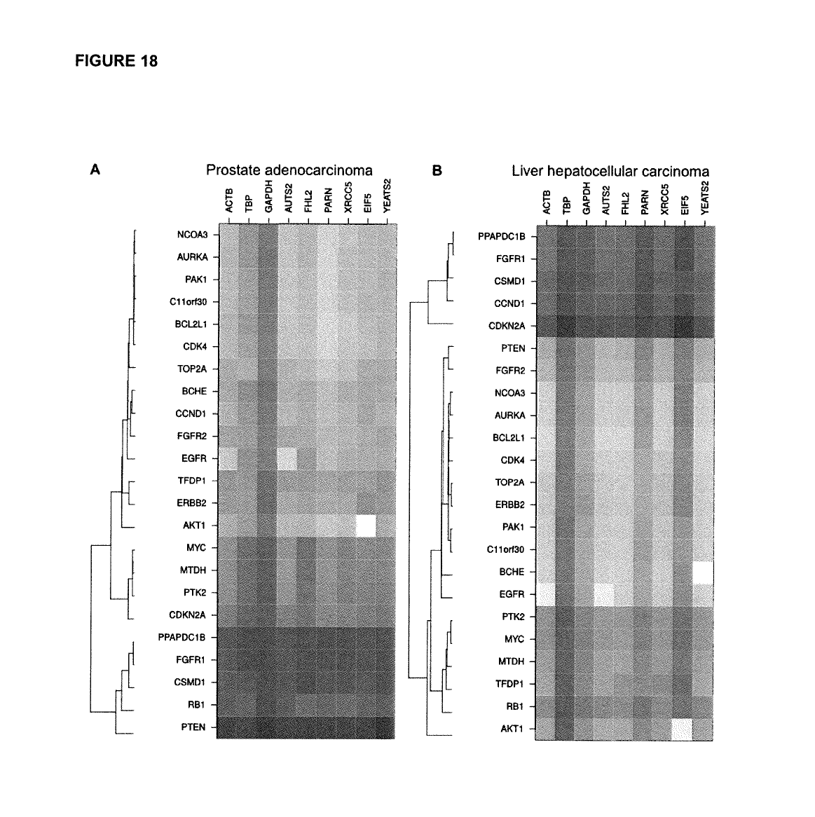

FIG. 18. Application of the present candidate loci can improve the Human Breast Cancer Copy Number PCR Array (Qiagen) applied to analysis of prostate adenocarcinoma (A) liver hepatocellular carcinoma (B). Each cell of the matrix displayed as a rectangular heat map (in each panel) represents expression a gene of interest (in rows) normalized by a given reference locus (in columns). The colour intensity in each cell represents the expression value (growing from white to black);

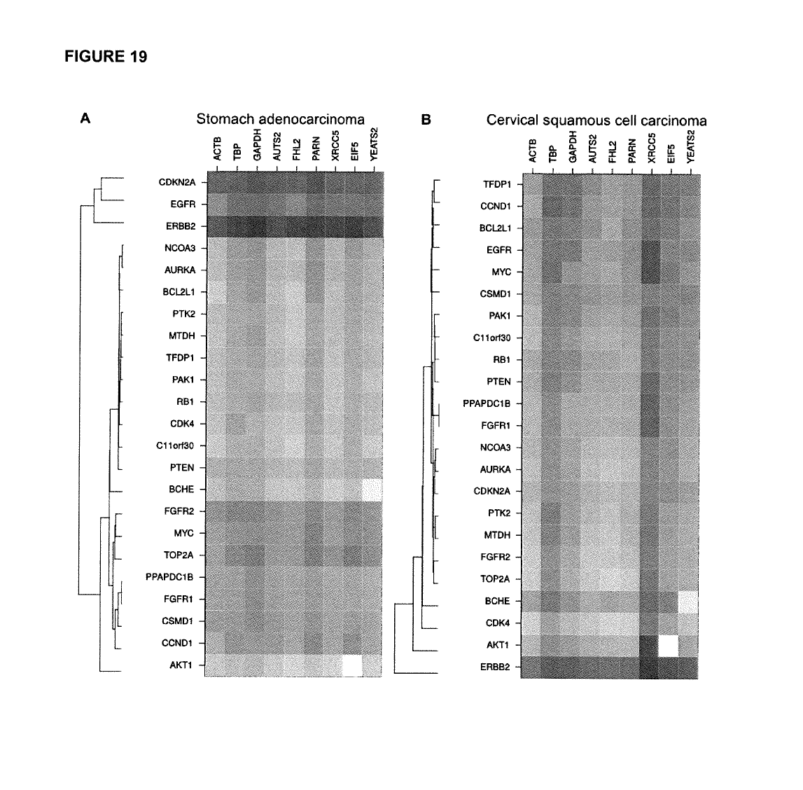

FIG. 19. Application of the present candidate loci can improve the Human Breast Cancer Copy Number PCR Array (Qiagen) applied to analysis of stomach adenocarcinoma (A) cervical squamous cell carcionma (B). Each cell of the matrix displayed as a rectangular heat map (in each panel) represents expression a gene of interest (in rows) normalized by a given reference locus (in columns). The colour intensity in each cell represents the expression value (growing from white to black);

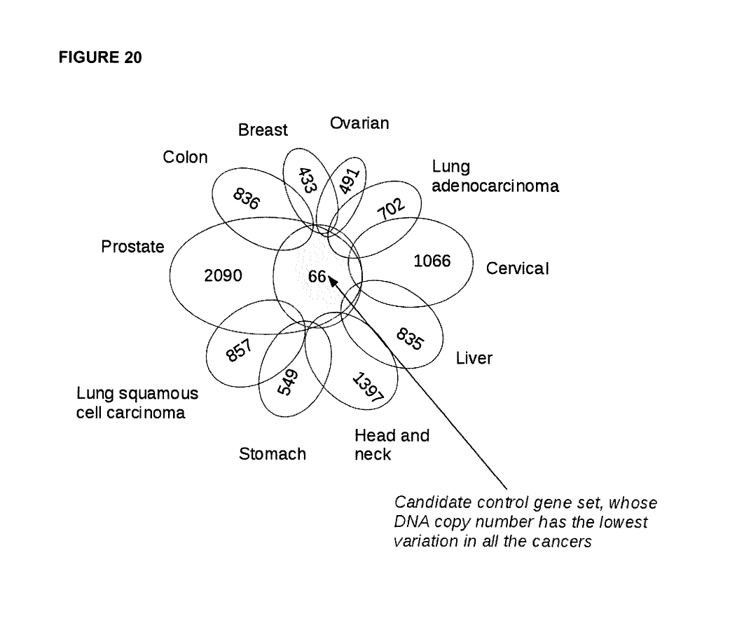

FIG. 20. The proposed method identified candidate normalization controls for DNA copy number measurements in the top 10 cancers. For each cancer a specific and a common set of loci are found and displayed as a Venn diagram; and

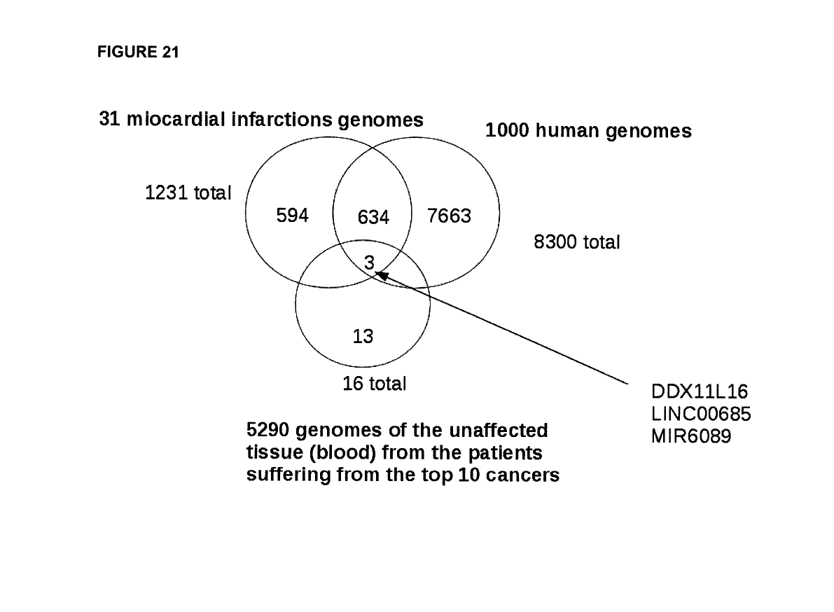

FIG. 21. An embodiment of the presently disclosed method identified candidate normalization controls for DNA copy number measurements in the non-cancerous samples from three cohorts: a) genomes of 1000 healthy humans, b) genomes of the blood cells collected as controls. Displayed as a Venn diagram.

### DEFINITIONS

### Biological Terms

For convenience, certain terms employed in the specification and examples are collected here.

The term “aptamer” is herein defined to be oligonucleotide acid or peptide molecule that binds to a specific target molecule. In particular, an aptamer used in the present invention may be generated using different technologies known in the art which include but is not limited to systematic evolution of ligands by exponential enrichment (SELEX) and the like.

The term “comprising” is herein defined to be that where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the method according to any aspect of the present invention “substantially” comprises the indicated step as an “essential” element. Additional steps may be included.

The term “difference” between two groups of patients is herein defined to be the statistical significance (p-value) of a partitioning of the patients within the two groups. Thus, achieving a “maximum difference” means finding a partition of maximal statistical significance (i.e. minimal p-value).

The term “label” or “label containing moiety” refers to a moiety capable of detection, such as a radioactive isotope or group containing same and non-isotopic labels, such as enzymes, biotin, avidin, streptavidin, digoxygenin, luminescent agents, dyes, haptens, and the like. Luminescent agents, depending upon the source of exciting energy, can be classified as radio luminescent, chemiluminescent, bio luminescent, and photo luminescent (including fluorescent and phosphorescent). A probe described herein can be bound, for example, chemically bound to label-containing moieties or can be suitable to be so bound. The probe can be directly or indirectly labelled.

The term “locus” is herein defined to be a specific location of a gene or DNA sequence on a chromosome. A variant of the DNA sequence at a given locus is called an allele.

The term “copy number (CN) value” or “DNA copy number value” is herein defined to refer to the number of copies of at least one DNA segment (locus) in the genome. The genome comprises DNA segments that may range from a small segment, the size of a single base pair to a large chromosome segment covering more than one gene. This number may be used to measure DNA structural variations, such as insertions, deletions and inversions occurring in a given genomic segment in a cell or a group of cells. In particular, the CN value may be determined in a cell or a group of cells by several methods known in the art including but not limited to comparative genomic hybridization (CGH) microarray, qPCR, electrophoretic separation and the like. CN value may be used as a measure of the copy number of a given DNA segment in a genome. In a single cell, the CN value may be defined by discrete values (0, 1, 2, 3 etc.). In a group of cells it may be a continuous variable, for example, a measure of DNA fragment CN ranging around 2 plus/minus increment d (theoretically or empirically defined variations). This number may be larger than 2+d or smaller than 2-d in the cells with a gain or loss of the nucleotides in a given locus, respectively.

With respect to associations between disease and CN value, a level of variation (deviation) in a DNA segment CN might be important. A level of positive or negative increment of the CN from normal dynamical range in a DNA sample of a given cell group or a single cell may be called CN variation.

The term “sample” is herein defined to include but is not limited to be blood, sputum, saliva, mucosal scraping, tissue biopsy and the like. The sample may be an isolated cell sample which may refer to a single cell, multiple cells, more than one type of cell, cells from tissues, cells from organs and/or cells from tumors.

A person skilled in the art will appreciate that the present invention may be practiced without undue experimentation according to the method given herein. The methods, techniques and chemicals are as described in the references given or from protocols in standard biotechnology and molecular biology text books.

The method according to any aspect of the present invention may be in vitro, or in vivo. In particular, the method may be in vitro, where the steps are carried out on a sample isolated from the subject. The sample may be taken from a subject by any method known in the art. By way of non-limiting example, ovarian tumor material may be extracted from ovaries, fallopian tubes, uterus, vagina and the like. Metastatic tumor samples may be extracted from the peritoneal cavity, other body organs, tissues and the like. Cancer cells may be extracted from non-limiting examples such as biological fluids, which include but are not limited to peritoneal liquid, blood, lymph, urine, products of body secretion and the like.

The term “genomic object” here defines a physical element of a given genome. Examples of a genomic object include (but are not limited to) a chromosome, a chromosomal arm, a plasmid.

The term “locally CN-invariant gene/locus” here defines a gene/locus with the number of copies, averaged across the span of the genomic coordinates of said gene/locus, staying unchanged under any extension of the locus' span within the entire genomic object.

The term “CN-invariant genes/loci in pathological samples”, or pathologically CN-invariant, here defines the genes/loci with average two copies per genome in pathological samples. The pathological samples can be represented by HG-SOC samples. A set of such genes/loci is listed in Table 1.

The term “CN-invariant genes/loci in normal tissues”, or biologically CN-invariant, here defines the genes/loci with average two copies per genome in tissue samples obtained from healthy humans. These samples can be represented by the ones collected in the Thousand Genomes project, for example. A set of such genes/loci is listed in Table 2.

The term CN-invariant genes/loci in human genome here defines the genes/loci being CN-invariant in both pathological and normal tissue samples. A set of such genes/loci is listed in Table 3.

The terms ‘invariant’ and ‘lowest variance’ here are used interchangeably for any data (including, but not limited to gene expression and copy number measurements), where variation across sample groups is not detected.

The terms ‘gene’ and ‘locus’ may be used interchangeably in the cases when the gene expression measurements are uncertain or irrelevant, for example when it is desired to quantify copy number but not gene expression.

The term genomic partition here defines a locus that includes the genomic coordinates of more than one gene.

The term cytoband here defines a genomic region that can be revealed by a standard cytogenetic staining (such as Giemsa staining).

The term human reference genome here defines the sequence annotated as the reference by the Genome Reference Consortium [Church D M, et al., PLoS Biology 9: 1001091 (2011)].

### Statistical Methods and Terms

The term “group of biological samples” is here defined as a collection of samples sharing one or more common biological or clinical property. Examples of such properties include (but are not limited to) tissue type, type of cells, source organism, the age of source organism, conditions of cellular growth, environmental conditions, treatment type.

The term normalization function here defines a function taking two arguments (the target and the reference), and returning one value. The function returns the scaling of the target in the units of the reference. The reference may be a single value or a set of values. An example of a normalization function is the ratio of the target value to the reference value. Standard score is an example of a normalization function, where the target is a single value, and the reference is a set: the standard score returns a scaling which is the ratio of the difference between the target value and the mean reference value to the standard deviation of the reference values.

The term normalization here defines a procedure of adjusting the values of the target measurement(s) by the values of the reference measurement(s), referred to as the normalization factor(s), using a normalization function. Typically, the normalization factor is the scaling returned by the normalization function.

The term reference gene here defines a gene that can be used as a normalization reference to obtain measurements of the target gene that would increase the measurements' accuracy upon the normalization.

The term reference locus (plural—loci), also referred to as locus reference, here defines the genomic coordinate range that can be used as a normalization reference(s) for measurements of the target locus or gene that would increase the measurements' accuracy upon normalization.

The term CN-invariant locus reference, also referred to as CNILR, in a given biological sample is here defined as a locus, which is locally CN-invariant; or in a biological sample representing a given group of biological samples the term CN-invariant locus reference is here defined as a locus with a minimal coefficient of variation value of its CN values across said group.

The term CN-invariant survival-insignificant locus reference(s) (CNISILR) in a biological sample representing a given group of biological samples, is defined as a CNILR, whose CN value, or any expression value of the genes within the locus, cannot define more than one subgroup of said group, based on survival prediction analysis.

The term numeric integrative measure here defines a function that takes a set of numeric values as an input and returns a single numeric value as an output. Examples of integrative measures are: mean, median, variance, maximum values.

The term robust measure is here defined as a measure, whose value does not significantly change if outliers are added to the measured data. Robustness of a measure may be defined for a specific measure compared to alternative measures of the same data (e.g. median vs. mean value estimation), or for a class of measures, compared to other classes of measures (e.g. a gene expression value measure with qPCR versus a gene expression microarray).

The term disease status information is here defined as a qualitative or quantitative variable defined for a patient (or a healthy subject) respective to a given disease, e.g. diagnosis, survival status (living or deceased) over a fixed time period, risk group, type of response to therapy, time after first disease recurrence. The particular value of a disease status information variable is here defined as the disease status.

The term disease status-significant genes is here defined as such genes that can stratify a cohort of patients into two or more groups by their given disease status with a given degree of statistical significance.

### EXAMPLES

### Example 1

Most of the genes in the genomes of EOC tumors (TCGA) are affected by CNV (FIG. 1). For example, the CNV distribution across in Chromosome 1 (FIG. 2) indicates that unlike the normal tissue control (fallopian tubes), EOC tumors at any stage of the disease include cells whose genomes carry numerous regions with CNV. Every chromosome and almost every tumor is affected.

The genomic regions unaffected by CNV typically spanned for a few megabases. The 851 cytobands containing no CNV, were selected as CN-invariant. The loci (obtained as the genomic coordinates of the longest transcription variants of the respective genes in the RefSeq database) affected by CNV were discarded, and 2841 unaffected genes were selected for further analysis. Among these genes, only 246 located in the CN-invariant cytobands (listed in Table 1). Such genes were considered CN-invariant. These loci and genes could serve as references for CNV measurement in EOC tumor samples.

To find such CN-invariant genes, which could be used as reference genes for both CNV and gene expression measurements, their median expression value and variance had to be assessed. For 157 of these loci (listed in Tables 2 and 3) Affymetrix U133A probes measured the expression of genes located in their genomic coordinates. These genes were considered CN-invariant and were tested for their expression median magnitude and variance across two cohorts of EOC tumors (TCGA and GSE9899).

As an additional criterion of robustness, the gene expression was tested for the significance of their expression values for the survival of the patients, using 1DDg method [Motakis E, et al., IEEE Eng Med Biol Mag 28: 58-66 (2009)]. Potentially, the CN and expression of survival-significant genes might change depending on the subgroup of the patients or treatment options, as the tumors expressing such genes might be subjects of selection. For the TOGA data set 92 genes (whose expression was measured by 121 probesets) satisfied this criterion, while in the GSE9899 data the number of such genes was 82 (with 117 corresponding probesets). Among them, 48 genes (measured with 59 probesets) were insignificant for survival (P>0.05) in both data sets (Table 4).

### Example 2

Actin B (ACTB) is among the genes most widely used as a reference in gene expression measurements with qRT-PCR. However, in the samples where CNV is observed within ACTB, using it as a reference increases the observed variation in the observed values of the copy number and gene expression of assessed genes. The example indicates that in EOC samples all genes of Actin family are characterized with a strong CNV (FIG. 3).

### Example 3

Genes, like ACTB, most commonly used as references for gene expression in normal samples, cannot be used as such in EOC samples both in the context of gene expression and copy number measurements, due to their essential CNV. Instead, reference genes should be selected firstly, based on the criteria of the minimal (or absent) CNV in the studied samples. A method implementing such selection is a part of the present invention. Only the genes with no CNV localized in cytobands with non-varying copy number are selected as CNV-invariant genes (FIG. 4). Additionally, the genes whose expression are high and correlate across two EOC cohorts (FIG. 5) are selected from the former list, as satisfying the criteria of both low CNV and high expression. The genes whose expression reveal a survival significance in any of the two studied patient cohorts, were excluded from the candidate reference gene list as potentially subjected to selective pressure.

### Example 4

The processed DCHGV (A Deep Catalog of Human Genetic Variation, 1000 Genomes Project) [Abecasis G R, et al., Nature 467: 1061-1073 (2010); Mills R E, et al., Nature 470: 59-65 (2011)] data set containing 89076 frequent gain/loss genomic aberrations in 19354 genes across 1062 samples was used in the analysis. Genes located in CN-invariant cytobands (i.e. cytobands contained no genomic gains or losses) in EOC tumors (TCGA) were filtered through the list of genes with aberrations obtained from the DCHGV. The 41 genes found to be CN-invariant in the TCGA EOC samples, and whose CN at the same time seldomly changed across the 1062 samples of normal human tissues, were considered CN-stable.

### Example 5

To validate the genes selected as CN-invariant in EOC tumors along with the algorithms for selection of such genes, the copy number and expression of a selected set of genes were measured with qRT-PCR in EOC tumors and normal tissues. The list of targets for validation included three genes most often used as expression references for qPCR experiments (ACTB, TBP, and GAPDH) and six genes obtained by using the algorithms described here (AUTS2, EIF5, FHL2, PARN, and YEATS2).

Two sets of primers were designed to detect the amplification of each of these genes in the qPCR reactions measuring either the CN or the expression values (Table 6). For further analyses primer set 2 was used. The primer melting curves demonstrate that all the primers have a single region of annealing in the human genome. Except for XRCC5, each primer pair demonstrates a single melting temperature within 75 to 90 degrees Celsius range (FIG. 6). The existence of additional small-scale melting events in the XRCC5 primer pair could be explained by a secondary structure in one or both primers of the pair. This effect is commonly considered insignificant for the primer specificity and sensitivity. To test the reproducibility of the obtained qPCR signal, the CN (FIGS. 7 and 9) and expression (FIGS. 8 and 10) of the reference genes were tested. The results show that both in both types of measurements the proposed reference genes were not less reproducible than the genes traditionally used as gene expression references (ACTB, GAPDH, and TBP).

### Example 6

To find whether the any of the traditional gene expression reference genes (ACTB, GAPDH, and TBP) could serve also as references for gene CN measurements, their CN distribution was evaluated across EOC tumor samples (TCGA cohort). The results demonstrate that CNV in these genes occur in 20 to 100 percent tumors, GAPDH tending to be amplified, and TBP to be deleted (FIG. 11).

To assess the effect of the reference genes, the CN of MECOM locus (one of the most frequently amplified in EOC) was normalized by the CN of the reference genes. It would some aspects of a CN measurement with a qPCR-based technique, where the CT values of the target gene is normalized by the CT values of the reference gene (FIG. 12). The results demonstrate that replacing ACTB with XRCC5 as a CN normalization reference increased the observed difference between the median MECOM CN in the tumors and the control samples (FIGS. 12A,D,F), decreased its variation in the tumor samples (FIG. 12B), and remained low in the tumor samples. For ACTB, EIF5, and XRCC5 the difference between the tumor and the control sample groups was significant (P<0.05, Wilcoxon test; FIG. 12A). For AUTS2 a borderline significance (P=0.06) was observed.

### Example 7

Ten most common cancers (Table 7), whose combined frequency account for 59% of all cancer cases worldwide, were selected, cross-validation of the loci serving as potential references for the Therascreen EGFR EGQ PCR kit (Qiagen). The six candidate reference loci proposed for ovarian cancer (see Table 6) were compared against ACTB, TBP, and GAPDH as potential normalization controls for the EGFR gene CN measurement (FIG. 13). The results demonstrate that in 8 out of 10 most common cancers (all, except for the colon and cervical cancers, thus comprising over 50% of all cancer cases) the lowest variation of the EGFR CN measurement is obtained with normalization by one of the proposed reference genes, but not ‘traditional reference genes’. The 2 cases, where the ‘traditional references’ (specifically, ACTB) perform better are cervical squamous cell carcinoma and colon cancer. For 7 of 10 cases, the reference gene with the worst performance was among the ‘traditional reference genes’. For the lung adenocarcinoma samples, the normalization by all the candidate reference loci resulted in the EGFR variation to be lower than in the cases for any of the traditional control loci. For the ovarian serous adenocarcinoma samples, the median variation across values obtained by the candidate reference loci was more than two times lower than that obtained by the traditional control loci.

### Example 8

Ten most common cancers (Table 7), whose combined frequency account for 59% of all cancer cases worldwide, were selected cross-validation of the loci serving as potential references for the Human Breast Cancer PCR array (Qiagen). The six candidate reference loci proposed for ovarian cancer (see Table 6) were compared against ACTB, TBP, and GAPDH as potential normalization controls for the CN measurements of the 23 diagnostic array loci (Table 12). Across the breast invasive carcinoma (FIG. 14A) and lung adenocarcinoma tumors (FIG. 14B), the lowest variation was revealed by one of the candidate reference loci for, at least, 22 out of the 23 loci of the diagnostic panel. When the median CN values across all the 23 panel loci were considered (FIG. 15), the results qualitatively recapitulated the ones obtained with EGFR EGQ kit (in the Example 7) by demonstrating that in 8 out of 10 most common cancers the median variation across the test loci CN measurements was lower, when normalized by one of the ovarian cancer candidate reference loci, compared with any of the traditional control loci (ACTB, TBP, and GAPDH).

For the lung adenocarcinoma (FIG. 14B) and ovarian serous adenocarcinoma (FIG. 18A), for all 23 assay loci, the normalization by at least one of the candidate reference loci resulted in the assay loci variation to be lower than in the cases when any of the traditional control loci were used. For the ovarian serous adenocarcinoma samples, the median variation across values obtained by the candidate reference loci was more than two times lower than that obtained by the traditional control loci.

Across the breast invasive carcinoma (FIG. 14A), lung squamous cell carcinoma (FIG. 16B), head and neck squamous cell carcinoma (FIG. 16A), and prostate adenocarcinoma (FIG. 18A), for, at least, 22 loci of the diagnostic panel, the lowest variation of the assay loci was obtained by using one of the candidate reference loci, but not the traditional control loci. For liver hepatocellular carcinoma (FIG. 18A) and stomach adenocarcinoma (FIG. 19A) the respective improvement was detected for 20 assay loci. For colon adenocarcinoma (FIG. 17B) and cervical squamous cell carcinoma (FIG. 19B) the improvement was detected for 15 and 14 assay loci, respectively.

### Example 9

An embodiment of the proposed method has been applied to select the candidate loci that could serve as common references to the ten most frequent cancers (Table 7) as follows. First, the loci with the lowest CN variation across the samples of each out of ten cancers (FIG. 20) were identified. Thus, ten loci lists were selected. Next, the loci common across all the ten lists, 66 loci (Table 8 and FIG. 20) were chosen as the reference candidates that can be used for normalization of the samples belonging to any of the ten selected cancers.

### Example 10

An embodiment of the proposed method has been applied to select the candidate loci that could serve as common references for tissues from healthy subjects, patients with non-cancerous disease, and cancer-unaffected tissues obtained from cancer patients. The healthy subjects were represented by the 1000 genomes of DCHGV cohort [Abecasis G R, et al., Nature 467: 1061-1073 (2010); Mills R E, et al., Nature 470: 59-65 (2011)] obtained from various tissues. The genomes of the non-cancerous patients were represented by the blood samples of 31 myocardial infarction patients (data set GSE31276).

To assess the CNV in the genomes of the 5290 patients, affected by the 10 most frequent cancers (listed in Table 7), genomic data of Level 3 (as defined by the TCGA data processing methods) was obtained. Each patient was characterized with the genomic data obtained from a pair of a blood sample and a tumor sample. Analyses of the tumor samples of these patients are presented in the Examples 7-9 (the TCGA cohort).

The blood samples of these patients were considered as cancer-unaffected, along with the samples from the DCHGV and the GSE31276 cohorts. Our analysis demonstrated that the total number of loci with the lowest, effectively zero, variation in were 8300, 1231, and 16 loci in the DCHGV, the GSE31276, and the TCGA cohorts, respectively (Table 9; FIG. 21). These three respective loci sets were suggested as cohort-specific sources of the reference loci.

In the intersections of these three sets, cross-cohort sources of reference loci were identified. A total of 637 loci revealed the lowest variance across both the DCHGV and the myocardial infarction patients' blood genomes, were considered as reference control candidates for non-cancerous genomes (Table 10).

Thee loci (Table 11) are most stable across normal subject, non-cancerous disease subject, and cancer-unaffected tissues of cancer patients. They are regarded as candidate reference loci for CN normalization across all non-cancerous subjects.

Altogether, the cohort-specific and cross-cohort reference loci might be applied to study naturally occurring DNA copy number variations in the blood. These variations might be population-specific and reveal markers of various disease predispositions.

The present invention developed from work on DNA quantification with qPCR. The quantification procedure requires knowledge of both the target locus (or gene) of interest and the locus (or gene) of reference. The DNA of the target locus is quantified by the difference between the PCR amplification cycles counts of the target gene and the reference gene. The main assumption of the method is that for the reference gene the DNA copy number (and hence the PCR amplification cycles count) remains the same for all samples, including the tested and the control ones. In our work we found that this assumption does not hold true for, at least, cancer samples. Since the cancer genome is highly mobile, and its evolution is unpredictable, any gene in the genome can be either amplified or deleted in a large number of cells comprising the cancer cells population. We experimentally observed that this amplification results in highly varying DNA copy numbers of the traditional qPCR reference loci, ACTB and GAPDH. Therefore, we experimentally confirmed that the above assumption is invalid. Moreover, since the RNA level of a gene is a product of the DNA of the same gene (with a non-linear dependence of the former on the latter), the validity of any universal standard loci for RNA quantification is also compromised.

To select a locus suitable as a qPCR reference, we proposed to discard the assumption of a universal reference, and developed procedures that would identify the best reference for a given multitude of samples. For example, the multitude may be defined as ovarian cancer samples (such as in Examples 1, 2, and 3). If we define that the best reference locus (or gene) is a locus, whose DNA copy number value, as measured in a given qPCR setup, simultaneously satisfies two or more conditions: 1) has the smallest variation in all the samples (the specificity criterion), 2) can be detected in all the samples, and/or 3) should not evolve with time or as a result of environmental condition changes (e.g. disease treatments). In patients, the third condition can be ensured by neutrality of the gene's copy number and expression to the patient survival. Thus, the definition of the best reference set dictates the criteria for an unbiased selection of the reference genes. We implemented a computational pipeline (FIGS. 4 and 5) that allowed us to scan through publicly available data on ovarian cancer samples and select a list of such candidate reference loci (given in Table 1; see also Example 1).

We carried out an experimental study to check whether the present most popular control loci (ACTB, GAPDH, TBP) satisfy the above conditions and how they compare to the list (Table 1) obtained with our unbiased selection method (see Example 5). We confirmed that: 1) the universal reference assumption does not hold true, since both ACTB and GAPDH reveal DNA copy number variation (FIGS. 3 and 11); 2) the unbiased search for ovarian cancer-specific reference loci provided the candidates, which satisfy the above reference criteria better than the TBP locus (Tables 1 and 4; FIGS. 7, 9, 11); 3) our method provides the best reference loci not only for DNA copy number (qPCR), but also expression measurements (Tables 2 and 3; FIGS. 8 and 10). To check these results in a real case scenario, we used our candidate reference loci, along with the traditional reference loci (ACTB, GAPDH, and TBP) to measure the DNA copy number and expression of the EVI1 gene of the MECOM complex locus (Example 6). We concluded that using of our candidate loci as references resulted in lower variations MECOM DNA copy number and RNA expression measurements, compared to the case, when the traditional reference loci were used (Example 6; FIG. 12). We also concluded that our experimental result validate our use of publicly available high-throughput data sets as the entry points for our computational pipeline.

To further predict the performance of our tests for the cases of other cancers and non-cancerous diseases, we carried out a computational study using publicly available high-throughput data obtained from patients diagnosed with ten most common cancer types (Examples 7, 8, and 9), myocardial infarction (Example 10), and a selection of healthy DNA donors from multiple populations across the world (Example 10). We also demonstrated how application of our method can improve the variability of the measurements obtained with two popular in-vitro diagnostic tests (Examples 7 and 8; FIGS. 13-20).

### Materials and Methods

### CGH Microarray Data Analysis

The publicly available Affymetrix SNP-6.0 microarray data (described in the Clinical data section) was retrieved from the Gene Expression Omnibus (GEO) repsitory. Each data set was independently normalized using the following steps:

### Clinical Data.

The initial data analysis was carried out with publicly available datasets: TCGA (The Cancer Genome Atlas) [Bell D, et al., Nature 474: 609-15 (2011)], GSE9899 [Tothill R W, et al., Clin Cancer Res 14: 5198-5208 (2008)], and DCHGV (A Deep Catalog of Human Genetic Variation, 1000 Genomes Project) [Abecasis G R, et al., Nature 467: 1061-1073 (2010); Mills R E, et al., Nature 470: 59-65 (2011)].

The National Institute of Health (NIH) Cancer Genome Atlas (TCGA) data set with 514 EOC patients was used for the analysis of CNV, gene expression and patient survival [Bell D, et al., Nature 474: 609-15 (2011)]. The patients, which EOC tumors had EVI1 gene amplified (average EVI1 gene copy number not less than 2.5 per cell), defined here as ‘EVI1 amplified group, were analyzed separately. The 5-year survival for this group of patients was 36 percent. The 5-year survival of the whole patient cohort was 28 percent. The 2-year survival of the whole patient cohort was 74 percent. Gene expression was measured with Affymetrix U133-A microarrays. Copy number was measured with Affymetrix SNP-6.0 CGH microarrays.

Gene Expression Omnibus (NIH) repository was used to obtain the GSE9899 (accession number) data set containing 246 samples [Tothill R W, et al., Clin Cancer Res 14: 5198-5208 (2008)]. From this set 16 patients were removed after a quality control assessment. The 5-year survival of the whole patient cohort was 44 percent. The 2-year survival of the whole patient cohort was 57 percent. Gene expression was measured with Affymetrix U133-Plus-2.0 microarrays.

A Deep Catalog of Human Genetic Variation (DCHGV) was used to obtain data on 202430 natural variations in the human genome reported in 10692 normal human tissue samples. Only variations reported as genomic gains or losses in more than 10 samples at frequencies more than 10% were included in the analysis. In total, 89076 genetic variations were selected, including 24891 cases of genomic gains and 64185 losses in 19354 genes, across 10692 biological samples.

Gene Expression Omnibus (NIH) repository was used to obtain the GSE31276 data set containing 31 individual genome profiles obtained from the blood of myocardial infarction patients. The samples were collected according to the Prospective Cardiovascular Munster study [Assmann G and Schulte H American heart journal 116: 1713-24 (1988)] and Framingham Heart study [Benjamin E J, et al., Circulation 98: 946-52 (1998)].

For validation experiments 48 DNA samples and 80 RNA samples purchased from Origene were used. The 48 DNA samples were extracted from individual serous ovarian adenocarcinoma tumors obtained from: 4 patients with the disease at stage 1, 3 patients at stage 2, 34 patients at stage 3, and 2 patients at stage 4. The 80 RNA samples were extracted from 7 normal fallopian tubes, 21 normal ovaries, and 52 individual serous ovarian adenocarcinoma tumors. The tumors were obtained from 11 patients with the disease at stage 1, 7 patients at stage 2, 29 patients at stage 3, and 5 patients at stage 4. For all 80 RNA samples the cDNA was synthesized using QuantiTect Reverse Transcription Kit 200 (Qiagen; cat. no: 205313).

### Tables

TABLE 1
Genes with invariant copy numbers across TCGA cohorts

| Symbol | Refseq | Chr | Start | End | Description |
|---|---|---|---|---|---|
| ABCB4 | NM_018849 | chr7 | 87031360 | 87105019 | multidrug resistance |
| | | | | | protein 3 isoform B |
| ABHD5 | NM_016006 | chr3 | 43732374 | 43764217 | 1-acylglycerol-3- |
| | | | | | phosphate O- |
| | | | | | acyltransferase ABHD5 |
| ACYP2 | NM_138448 | chr2 | 54342409 | 54532435 | acylphosphatase-2 |
| AFF3 | NM_001025108 | chr2 | 100163715 | 100722045 | AF4/FMR2 family |
| | | | | | member 3 isoform 2 |
| AGAP1 | NM_001244888 | chr2 | 236402732 | 236761846 | arf-GAP with GTPase, |
| | | | | | ANK repeat and PH |
| | | | | | domain-containing |
| | | | | | protein 1 isoform 3 |
| AGBL4 | NM_032785 | chr1 | 48998526 | 50489626 | cytosolic |
| | | | | | carboxypeptidase 6 |
| AMD1 | NM_001287216 | chr6 | 111195986 | 111216915 | S-adenosylmethionine |
| | | | | | decarboxylase |
| | | | | | proenzyme isoform 5 |
| ANK2 | NM_001127493 | chr4 | 113739238 | 114304896 | ankyrin-2 isoform 3 |
| ARSE | NM_001282628 | chrX | 2852672 | 2882494 | arylsulfatase E isoform |
| | | | | | 1 |
| ASAP1 | NM_018482 | chr8 | 131064350 | 131455906 | arf-GAP with SH3 |
| | | | | | domain, ANK repeat |
| | | | | | and PH domain- |
| | | | | | containing protein 1 |
| | | | | | isoform 1 |
| ASCC3 | NM_001284271 | chr6 | 101163006 | 101329248 | activating signal |
| | | | | | cointegrator 1 complex |
| | | | | | subunit 3 isoform c |
| ATAD2B | NM_001242338 | chr2 | 23971533 | 24149984 | ATPase family AAA |
| | | | | | domain-containing |
| | | | | | protein 2B isoform 2 |
| ATF7IP2 | NM_024997 | chr16 | 10479911 | 10577495 | activating transcription |
| | | | | | factor 7-interacting |
| | | | | | protein 2 isoform 1 |
| ATXN7 | NM_001128149 | chr3 | 63953419 | 63989136 | ataxin-7 isoform c |
| AUTS2 | NM_015570 | chr7 | 69063904 | 70258054 | autism susceptibility |
| | | | | | gene 2 protein isoform 1 |
| AZIN2 | NM_052998 | chr1 | 33546713 | 33586132 | antizyme inhibitor 2 |
| | | | | | isoform 1 |
| BATF3 | NM_018664 | chr1 | 212859758 | 212873327 | basic leucine zipper |
| | | | | | transcriptional factor |
| | | | | | ATF-like 3 |
| BMPR2 | NM_001204 | chr2 | 203241049 | 203432474 | bone morphogenetic |
| | | | | | protein receptor type-2 |
| | | | | | precursor |
| BTLA | NM_001085357 | chr3 | 112182812 | 112218408 | B- and T-lymphocyte |
| | | | | | attenuator isoform 2 |
| BTNL8 | NM_001159707 | chr5 | 180326076 | 180377906 | butyrophilin-like protein |
| | | | | | 8 isoform 3 precursor |
| C1orf21 | NM_030806 | chr1 | 184356149 | 184598155 | uncharacterized protein |
| | | | | | C1orf21 |
| C4orf22 | NM_001206997 | chr4 | 81256873 | 81884910 | uncharacterized protein |
| | | | | | C4orf22 isoform 1 |
| C4orf33 | NM_173487 | chr4 | 130014828 | 130033843 | UPF0462 protein |
| | | | | | C4orf33 |
| CACNB2 | NM_201571 | chr10 | 18429741 | 18830688 | voltage-dependent L- |
| | | | | | type calcium channel |
| | | | | | subunit beta-2 isoform 6 |
| CADM2 | NM_153184 | chr3 | 85775631 | 86123579 | cell adhesion molecule 2 |
| | | | | | isoform 3 precursor |
| CAMTA1 | NR_038934 | chr1 | 6845383 | 6948261 | |
| CASC5 | NM_170589 | chr15 | 40886446 | 40954881 | protein CASC5 isoform |
| | | | | | 1 |
| CASQ2 | NM_001232 | chr1 | 116242625 | 116311426 | calsequestrin-2 |
| | | | | | precursor |
| CCDC88A | NM_018084 | chr2 | 55514977 | 55647057 | girdin isoform 2 |
| CHL1 | NR_045572 | chr3 | 239325 | 290282 | |
| CHST15 | NM_014863 | chr10 | 125779168 | 125851940 | carbohydrate |
| | | | | | sulfotransferase 15 |
| | | | | | isoform 2 |
| CLASP1 | NM_001142273 | chr2 | 122095351 | 122407052 | CLIP-associating |
| | | | | | protein 1 isoform 2 |
| CLIC4 | NM_013943 | chr1 | 25071759 | 25170815 | chloride intracellular |
| | | | | | channel protein 4 |
| CLMN | NM_024734 | chr14 | 95648275 | 95786245 | calmin |
| CNTN3 | NM_020872 | chr3 | 74311721 | 74570343 | contactin-3 precursor |
| COPA | NM_001098398 | chr1 | 160258376 | 160313354 | coatomer subunit alpha |
| | | | | | isoform 1 |
| CTTNBP2 | NM_033427 | chr7 | 117350705 | 117513561 | cortactin-binding |
| | | | | | protein 2 |
| CUL3 | NM_001257197 | chr2 | 225334866 | 225450114 | cullin-3 isoform 2 |
| DAB1 | NM_021080 | chr1 | 57463578 | 58716211 | disabled homolog 1 |
| DAPK1 | NM_001288729 | chr9 | 90113449 | 90323549 | death-associated protein |
| | | | | | kinase 1 |
| DDAH1 | NM_012137 | chr1 | 85784167 | 85930889 | N(G),N(G)- |
| | | | | | dimethylarginine |
| | | | | | dimethylaminohydrolase |
| | | | | | 1 isoform 1 |
| DEGS1 | NM_003676 | chr1 | 224370909 | 224381142 | sphingolipid delta(4)- |
| | | | | | desaturase DES1 |
| DEPDC1 | NM_001114120 | chr1 | 68939834 | 68962904 | DEP domain-containing |
| | | | | | protein 1A isoform a |
| DGAT2 | NM_001253891 | chr11 | 75479777 | 75512581 | diacylglycerol O- |
| | | | | | acyltransferase 2 |
| | | | | | isoform 2 |
| DNM3 | NM_015569 | chr1 | 171810617 | 172381857 | dynamin-3 isoform a |
| DPP10 | NM_001178034 | chr2 | 115919512 | 116602326 | inactive dipeptidyl |
| | | | | | peptidase 10 isoform c |
| DPPA4 | NM_018189 | chr3 | 109044987 | 109056419 | developmental |
| | | | | | pluripotency-associated |
| | | | | | protein 4 |
| DYRK1A | NM_001396 | chr21 | 38792601 | 38887679 | dual specificity tyrosine- |
| | | | | | phosphorylation- |
| | | | | | regulated kinase 1A |
| | | | | | isoform 1 |
| EFHC2 | NM_025184 | chrX | 44007127 | 44202923 | EF-hand domain- |
| | | | | | containing family |
| | | | | | member C2 |
| EHBP1 | NM_015252 | chr2 | 62933000 | 63273621 | EH domain-binding |
| | | | | | protein 1 isoform 1 |
| EHD3 | NM_014600 | chr2 | 31456879 | 31491260 | EH domain-containing |
| | | | | | protein 3 |
| EIF5 | NM_001969 | chr14 | 103800338 | 103811361 | eukaryotic translation |
| | | | | | initiation factor 5 |
| EMX2OS | NR_002791 | chr10 | 119243803 | 119304579 | |
| ENPP2 | NR_045555 | chr8 | 120569316 | 120605248 | |
| EPB41 | NM_001166007 | chr1 | 29213602 | 29446558 | protein 4.1 isoform 5 |
| EPHB2 | NM_004442 | chr1 | 23037330 | 23241823 | ephrin type-B receptor 2 |
| | | | | | isoform 2 precursor |
| ERBB4 | NM_005235 | chr2 | 212240441 | 213403352 | receptor tyrosine-protein |
| | | | | | kinase erbB-4 isoform |
| | | | | | JM-a/CVT-1 precursor |
| ERC2 | NM_015576 | chr3 | 55542335 | 56502391 | ERC protein 2 |
| ESRRG | NM_206594 | chr1 | 216676587 | 217262987 | estrogen-related |
| | | | | | receptor gamma isoform |
| | | | | | 2 |
| FAHD2A | NM_016044 | chr2 | 96068447 | 96078879 | fumarylacetoacetate |
| | | | | | hydrolase domain- |
| | | | | | containing protein 2A |
| FAM132B | NM_001291832 | chr2 | 239067648 | 239077532 | erythroferrone precursor |
| FAM135B | NM_015912 | chr8 | 139142265 | 139509065 | protein FAM135B |
| FAM49A | NM_030797 | chr2 | 16730729 | 16847134 | protein FAM49A |
| FAT1 | NM_005245 | chr4 | 187508936 | 187644987 | protocadherin Fat 1 |
| | | | | | precursor |
| FBXO32 | NM_058229 | chr8 | 124510126 | 124553493 | F-box only protein 32 |
| | | | | | isoform 1 |
| FCGR2A | NM_001136219 | chr1 | 161475204 | 161489360 | low affinity |
| | | | | | immunoglobulin gamma |
| | | | | | Fc region receptor II-a |
| | | | | | isoform 1 precursor |
| FGF12 | NM_004113 | chr3 | 191857181 | 192445388 | fibroblast growth factor |
| | | | | | 12 isoform 2 |
| FGGY | NM_001113411 | chr1 | 59762624 | 60228402 | FGGY carbohydrate |
| | | | | | kinase domain- |
| | | | | | containing protein |
| | | | | | isoform a |
| FHIT | NM_002012 | chr3 | 59735035 | 61237133 | bis(5′-adenosyl)- |
| | | | | | triphosphatase |
| FHL1 | NM_001159702 | chrX | 135229558 | 135293518 | four and a half LIM |
| | | | | | domains protein 1 |
| | | | | | isoform 1 |
| FHL2 | NM_201557 | chr2 | 105977282 | 106055230 | four and a half LIM |
| | | | | | domains protein 2 |
| FOXP1 | NM_001012505 | chr3 | 71247033 | 71633140 | forkhead box protein P1 |
| | | | | | isoform 2 |
| FRMD3 | NM_001244959 | chr9 | 85857904 | 86153348 | FERM domain- |
| | | | | | containing protein 3 |
| | | | | | isoform 2 |
| FUT9 | NM_006581 | chr6 | 96463844 | 96663488 | alpha-(1,3)- |
| | | | | | fucosyltransferase 9 |
| GADL1 | NM_207359 | chr3 | 30767691 | 30936153 | acidic amino acid |
| | | | | | decarboxylase GADL1 |
| GAP43 | NM_002045 | chr3 | 115342150 | 115440334 | neuromodulin isoform 2 |
| GBAP1 | NR_002188 | chr1 | 155183615 | 155197325 | |
| GBE1 | NM_000158 | chr3 | 81538849 | 81810950 | 1,4-alpha-glucan- |
| | | | | | branching enzyme |
| GLI2 | NM_005270 | chr2 | 121554866 | 121750229 | zinc finger protein GLI2 |
| GOLIM4 | NM_014498 | chr3 | 167727653 | 167813417 | Golgi integral |
| | | | | | membrane protein 4 |
| GPBP1L1 | NM_021639 | chr1 | 46092975 | 46152302 | vasculin-like protein 1 |
| GRM8 | NM_001127323 | chr7 | 126078651 | 126892428 | metabotropic glutamate |
| | | | | | receptor 8 isoform b |
| | | | | | precursor |
| GTF2F2 | NM_004128 | chr13 | 45694630 | 45858239 | general transcription |
| | | | | | factor IIF subunit 2 |
| H6PD | NM_001282587 | chr1 | 9299902 | 9331394 | GDH/6PGL |
| | | | | | endoplasmic |
| | | | | | bifunctional protein |
| | | | | | isoform 1 precursor |
| HHAT | NM_001122834 | chr1 | 210501595 | 210849638 | protein-cysteine N- |
| | | | | | palmitoyltransferase |
| | | | | | HHAT isoform 1 |
| HS3ST1 | NM_005114 | chr4 | 11399987 | 11430537 | heparan sulfate |
| | | | | | glucosamine 3-O- |
| | | | | | sulfotransferase 1 |
| | | | | | precursor |
| HTR4 | NM_199453 | chr5 | 147830594 | 148016624 | 5-hydroxytryptamine |
| | | | | | receptor 4 isoform g |
| HYAL3 | NM_003549 | chr3 | 50330258 | 50336899 | hyaluronidase-3 isoform |
| | | | | | 1 precursor |
| IDO2 | NM_194294 | chr8 | 39792473 | 39873910 | indoleamine 2,3- |
| | | | | | dioxygenase 2 |
| IGSF11 | NM_152538 | chr3 | 118619478 | 118864898 | immunoglobulin |
| | | | | | superfamily member 11 |
| | | | | | isoform a precursor |
| IL15 | NR_037840 | chr4 | 142557748 | 142655140 | |
| IL5RA | NM_175726 | chr3 | 3108007 | 3152058 | interleukin-5 receptor |
| | | | | | subunit alpha isoform 1 |
| | | | | | precursor |
| IQGAP3 | NM_178229 | chr1 | 156495196 | 156542396 | ras GTPase-activating- |
| | | | | | like protein IQGAP3 |
| KCNAB1 | NM_172159 | chr3 | 156008775 | 156256927 | voltage-gated potassium |
| | | | | | channel subunit beta-1 |
| | | | | | isoform 3 |
| KCNIP4 | NM_147183 | chr4 | 20730238 | 21305529 | Kv channel-interacting |
| | | | | | protein 4 isoform 4 |
| LAMC3 | NM_006059 | chr9 | 133884503 | 133968446 | laminin subunit gamma- |
| | | | | | 3 precursor |
| LDB2 | NM_001290 | chr4 | 16503164 | 16900424 | LIM domain-binding |
| | | | | | protein 2 isoform a |
| LEF1 | NM_001130714 | chr4 | 108968700 | 109090112 | lymphoid enhancer- |
| | | | | | binding factor 1 isoform |
| | | | | | 3 |
| LIN54 | NM_001115008 | chr4 | 83845756 | 83931987 | protein lin-54 homolog |
| | | | | | isoform b |
| LIN9 | NM_001270410 | chr1 | 226418849 | 226497449 | protein lin-9 homolog |
| | | | | | isoform 3 |
| LOC100506122 | NR_038838 | chr4 | 171961752 | 171980311 | |
| LOC100506457 | NR_110198 | chr2 | 12147241 | 12223743 | |
| LOC101926942 | NR_110657 | chr10 | 92162277 | 92300562 | |
| LOC101927905 | NR_120455 | chr12 | 8388010 | 8391553 | |
| LPHN3 | NM_015236 | chr4 | 62362838 | 62938168 | latrophilin-3 precursor |
| LRCH1 | NM_015116 | chr13 | 47127295 | 47319036 | leucine-rich repeat and |
| | | | | | calponin homology |
| | | | | | domain-containing |
| | | | | | protein 1 isoform 2 |
| LRP1B | NM_018557 | chr2 | 140988995 | 142889270 | low-density lipoprotein |
| | | | | | receptor-related protein |
| | | | | | 1B precursor |
| LRRC8C | NM_032270 | chr1 | 90098643 | 90185094 | volume-regulated anion |
| | | | | | channel subunit |
| | | | | | LRRC8C |
| LYST | NM_001301365 | chr1 | 235824330 | 236047008 | lysosomal-trafficking |
| | | | | | regulator |
| LZTS2 | NM_032429 | chr10 | 102756863 | 102767593 | leucine zipper putative |
| | | | | | tumor suppressor 2 |
| MALRD1 | NM_001142308 | chr10 | 19337699 | 20023407 | MAM and LDL- |
| | | | | | receptor class A |
| | | | | | domain-containing |
| | | | | | protein 1 precursor |
| MAN1A1 | NM_005907 | chr6 | 119498365 | 119670931 | mannosyl- |
| | | | | | oligosaccharide 1,2- |
| | | | | | alpha-mannosidase IA |
| MCHR2 | NM_001040179 | chr6 | 100367785 | 100442099 | melanin-concentrating |
| | | | | | hormone receptor 2 |
| MCTP1 | NM_001002796 | chr5 | 94041241 | 94417570 | multiple C2 and |
| | | | | | transmembrane domain- |
| | | | | | containing protein 1 |
| | | | | | isoform S |
| MFAP3L | NM_021647 | chr4 | 170907747 | 170947581 | microfibrillar-associated |
| | | | | | protein 3-like isoform 1 |
| | | | | | precursor |
| MIR5694 | NR_049879 | chr10 | 122344590 | 122806858 | |
| MORC3 | NM_015358 | chr21 | 37692486 | 37748944 | MORC family CW-type |
| | | | | | zinc finger protein 3 |
| MRPL47 | NM_020409 | chr3 | 179306254 | 179322434 | 39S ribosomal protein |
| | | | | | L47, mitochondrial |
| | | | | | isoform a |
| MTA1 | NM_001203258 | chr14 | 105886185 | 105937057 | metastasis-associated |
| | | | | | protein MTA1 isoform |
| | | | | | MTA1s |
| NAA16 | NM_024561 | chr13 | 41885340 | 41951166 | N-alpha- |
| | | | | | acetyltransferase 16, |
| | | | | | NatA auxiliary subunit |
| | | | | | isoform 1 |
| NBPF8 | NR_102404 | chr1 | 147574322 | 148346929 | |
| NCOA7 | NM_001199619 | chr6 | 126102306 | 126253176 | nuclear receptor |
| | | | | | coactivator 7 isoform 1 |
| NECAP2 | NM_001145278 | chr1 | 16767166 | 16786584 | adaptin ear-binding |
| | | | | | coat-associated protein 2 |
| | | | | | isoform 3 |
| NEGR1 | NM_173808 | chr1 | 71868624 | 72748277 | neuronal growth |
| | | | | | regulator 1 precursor |
| NEIL3 | NM_018248 | chr4 | 178230990 | 178284092 | endonuclease 8-like 3 |
| NLGN4X | NM_181332 | chrX | 5808066 | 6146923 | neuroligin-4, X-linked |
| NMD3 | NM_015938 | chr3 | 160939098 | 160969795 | 60S ribosomal export |
| | | | | | protein NMD3 |
| NOTCH2 | NM_024408 | chr1 | 120454175 | 120612317 | neurogenic locus notch |
| | | | | | homolog protein 2 |
| | | | | | isoform 1 preproprotein |
| NRP2 | NM_018534 | chr2 | 206547223 | 206641880 | neuropilin-2 isoform 4 |
| | | | | | precursor |
| NRXN1 | NM_004801 | chr2 | 50145642 | 51259674 | neurexin-1-beta isoform |
| | | | | | alpha 1 precursor |
| NT5C2 | NM_001134373 | chr10 | 104847773 | 104953063 | cytosolic purine 5′- |
| | | | | | nucleotidase |
| NTNG1 | NM_014917 | chr1 | 107682744 | 108024475 | netrin-G1 isoform 3 |
| | | | | | precursor |
| NUP133 | NM_018230 | chr1 | 229577043 | 229644088 | nuclear pore complex |
| | | | | | protein Nup133 |
| NYAP2 | NM_020864 | chr2 | 226265601 | 226518734 | neuronal tyrosine- |
| | | | | | phosphorylated |
| | | | | | phosphoinositide-3- |
| | | | | | kinase adapter 2 |
| OLFM3 | NM_058170 | chr1 | 102268122 | 102462790 | noelin-3 isoform 2 |
| | | | | | precursor |
| OSBPL5 | NM_145638 | chr11 | 3108345 | 3186582 | oxysterol-binding |
| | | | | | protein-related protein 5 |
| | | | | | isoform b |
| PARN | NM_001134477 | chr16 | 14529556 | 14724128 | poly(A)-specific |
| | | | | | ribonuclease PARN |
| | | | | | isoform 2 |
| PCDH10 | NM_020815 | chr4 | 134070469 | 134074404 | protocadherin-10 |
| | | | | | isoform 2 precursor |
| PCDH7 | NM_032456 | chr4 | 30722029 | 30726957 | protocadherin-7 isoform |
| | | | | | b precursor |
| PCOLCE2 | NM_013363 | chr3 | 142536701 | 142608045 | procollagen C- |
| | | | | | endopeptidase enhancer |
| | | | | | 2 precursor |
| PDE2A | NM_001146209 | chr11 | 72287183 | 72380108 | cGMP-dependent 3′,5′- |
| | | | | | cyclic |
| | | | | | phosphodiesterase |
| | | | | | isoform PDE2A4 |
| PDE6C | NM_006204 | chr10 | 95372344 | 95425429 | cone cGMP-specific |
| | | | | | 3′,5′-cyclic |
| | | | | | phosphodiesterase |
| | | | | | subunit alpha' |
| PDIA3 | NM_005313 | chr15 | 44038589 | 44064804 | protein disulfide- |
| | | | | | isomerase A3 precursor |
| PDZK1 | NM_001201325 | chr1 | 145727665 | 145764206 | Na(+)/H(+) exchange |
| | | | | | regulatory cofactor |
| | | | | | NHE-KF3 isoform 1 |
| PHTF1 | NM_006608 | chr1 | 114239823 | 114301777 | putative homeodomain |
| | | | | | transcription factor 1 |
| PLEKHA2 | NM_021623 | chr8 | 38758752 | 38831430 | pleckstrin homology |
| | | | | | domain-containing |
| | | | | | family A member 2 |
| POU2F1 | NM_001198783 | chr1 | 167298280 | 167396582 | POU domain, class 2, |
| | | | | | transcription factor 1 |
| | | | | | isoform 2 |
| PRDM16 | NM_022114 | chr1 | 2985741 | 3355185 | PR domain zinc finger |
| | | | | | protein 16 isoform 1 |
| PRDM5 | NM_001300824 | chr4 | 121613067 | 121844021 | PR domain zinc finger |
| | | | | | protein 5 isfoorm 3 |
| PRKCE | NM_005400 | chr2 | 45879042 | 46415129 | protein kinase C epsilon |
| | | | | | type |
| PRKCZ | NM_001033582 | chr1 | 2036154 | 2116834 | protein kinase C zeta |
| | | | | | type isoform 2 |
| PRUNE | NM_021222 | chr1 | 150980972 | 151008189 | protein prune homolog |
| | | | | | isoform 1 |
| PTGS2 | NM_000963 | chr1 | 186640943 | 186649559 | prostaglandin G/H |
| | | | | | synthase 2 precursor |
| PTPRF | NM_130440 | chr1 | 43996546 | 44089343 | receptor-type tyrosine- |
| | | | | | protein phosphatase F |
| | | | | | isoform 2 precursor |
| PTPRZ1 | NM_002851 | chr7 | 121513158 | 121702090 | receptor-type tyrosine- |
| | | | | | protein phosphatase zeta |
| | | | | | isoform 1 precursor |
| PUM1 | NM_014676 | chr1 | 31404352 | 31538564 | pumilio homolog 1 |
| | | | | | isoform 2 |
| RAD52 | NM_001297419 | chr12 | 1020901 | 1099207 | DNA repair protein |
| | | | | | RAD52 homolog |
| | | | | | isoform a |
| RAI2 | NM_001172743 | chrX | 17818168 | 17879457 | retinoic acid-induced |
| | | | | | protein 2 isoform 1 |
| RDH13 | NM_138412 | chr19 | 55555691 | 55580914 | retinol dehydrogenase |
| | | | | | 13 isoform 2 |
| RFWD2 | NM_022457 | chr1 | 175913961 | 176176380 | E3 ubiquitin-protein |
| | | | | | ligase RFWD2 isoform |
| | | | | | a |
| RGS18 | NM_130782 | chr1 | 192127591 | 192154945 | regulator of G-protein |
| | | | | | signaling 18 |
| RNF144A | NM_014746 | chr2 | 7057522 | 7184309 | E3 ubiquitin-protein |
| | | | | | ligase RNF144A |
| SCHIP1 | NM_014575 | chr3 | 158991035 | 159615155 | schwannomin- |
| | | | | | interacting protein 1 |
| | | | | | isoform 1 |
| SERTAD2 | NM_014755 | chr2 | 64858754 | 64881046 | SERTA domain- |
| | | | | | containing protein 2 |
| SGCZ | NM_139167 | chr8 | 13947372 | 15095792 | zeta-sarcoglycan |
| SGIP1 | NM_032291 | chr1 | 66999824 | 67210768 | SH3-containing GRB2- |
| | | | | | like protein 3-interacting |
| | | | | | protein 1 |
| SGPP2 | NM_152386 | chr2 | 223289321 | 223423617 | sphingosine-1- |
| | | | | | phosphate phosphatase 2 |
| SH3KBP1 | NM_001024666 | chrX | 19552082 | 19817917 | SH3 domain-containing |
| | | | | | kinase-binding protein 1 |
| | | | | | isoform b |
| SH3RF3 | NM_001099289 | chr2 | 109745996 | 110262213 | SH3 domain-containing |
| | | | | | RING finger protein 3 |
| | | | | | precursor |
| SLC12A6 | NM_001042495 | chr15 | 34522196 | 34630265 | solute carrier family 12 |
| | | | | | member 6 isoform c |
| SLC15A2 | NM_001145998 | chr3 | 121613170 | 121663034 | solute carrier family 15 |
| | | | | | member 2 isoform b |
| SLC30A8 | NM_001172815 | chr8 | 117963189 | 118188953 | zinc transporter 8 |
| | | | | | isoform b |
| SLC45A1 | NM_001080397 | chr1 | 8378144 | 8404227 | proton-associated sugar |
| | | | | | transporter A |
| SLC4A4 | NM_003759 | chr4 | 72204769 | 72437804 | electrogenic sodium |
| | | | | | bicarbonate |
| | | | | | cotransporter 1 isoform |
| | | | | | 2 |
| SMYD3 | NM_022743 | chr1 | 245912641 | 246580714 | histone-lysine N- |
| | | | | | methyltransferase |
| | | | | | SMYD3 isoform 2 |
| SNTG2 | NM_018968 | chr2 | 946553 | 1371384 | gamma-2-syntrophin |
| SPATS2L | NM_001100424 | chr2 | 201170984 | 201346986 | SPATS2-like protein |
| | | | | | isoform b |
| SRGAP2C | NM_001271872 | chr1 | 206516199 | 206581301 | SLIT-ROBO Rho |
| | | | | | GTPase-activating |
| | | | | | protein 2C |
| STARD9 | NM_020759 | chr15 | 42867856 | 43013196 | stAR-related lipid |
| | | | | | transfer protein 9 |
| SYTL5 | NM_001163334 | chrX | 37892786 | 37988073 | synaptotagmin-like |
| | | | | | protein 5 isoform 2 |
| TBL1X | NM_001139468 | chrX | 9431334 | 9687780 | F-box-like/WD repeat- |
| | | | | | containing protein |
| | | | | | TBL1X isoform b |
| TC2N | NM_152332 | chr14 | 92246095 | 92302870 | tandem C2 domains |
| | | | | | nuclear protein isoform |
| | | | | | 1 |
| TCEANC2 | NM_153035 | chr1 | 54519273 | 54565416 | transcription elongation |
| | | | | | factor A N-terminal and |
| | | | | | central domain- |
| | | | | | containing protein 2 |
| TENM3 | NM_001080477 | chr4 | 183245136 | 183724177 | teneurin-3 |
| TEX41 | NR_033870 | chr2 | 145425533 | 145834291 | |
| TGFBR3 | NM_001195683 | chr1 | 92145899 | 92351836 | transforming growth |
| | | | | | factor beta receptor type |
| | | | | | 3 isoform b precursor |
| THRAP3 | NM_005119 | chr1 | 36690016 | 36770957 | thyroid hormone |
| | | | | | receptor-associated |
| | | | | | protein 3 |
| TIAM1 | NM_003253 | chr21 | 32490735 | 32931290 | T-lymphoma invasion |
| | | | | | and metastasis-inducing |
| | | | | | protein 1 |
| TLE4 | NM_007005 | chr9 | 82186687 | 82341796 | transducin-like enhancer |
| | | | | | protein 4 isoform 3 |
| TMEM236 | NM_001098844 | chr10 | 18041226 | 18089854 | transmembrane protein |
| | | | | | 236 |
| TNIK | NM_001161561 | chr3 | 170780291 | 171178197 | TRAF2 and NCK- |
| | | | | | interacting protein |
| | | | | | kinase isoform 3 |
| TPTE2P6 | NR_002815 | chr13 | 25154345 | 25171812 | |
| TRIM48 | NM_024114 | chr11 | 55029657 | 55038595 | tripartite motif- |
| | | | | | containing protein 48 |
| TRPM8 | NM_024080 | chr2 | 234826042 | 234928166 | transient receptor |
| | | | | | potential cation channel |
| | | | | | subfamily M member 8 |
| TRUB2 | NM_015679 | chr9 | 131071395 | 131084697 | probable tRNA |
| | | | | | pseudouridine synthase |
| | | | | | 2 |
| TSPAN9 | NM_001168320 | chr12 | 3186520 | 3395730 | tetraspanin-9 |
| TTC29 | NM_031956 | chr4 | 147628178 | 147867034 | tetratricopeptide repeat |
| | | | | | protein 29 isoform 2 |
| TTC7B | NM_001010854 | chr14 | 91006931 | 91282761 | tetratricopeptide repeat |
| | | | | | protein 7B |
| TTF1 | NM_001205296 | chr9 | 135250936 | 135282238 | transcription termination |
| | | | | | factor 1 isoform 2 |
| VPS8 | NM_015303 | chr3 | 184529930 | 184770402 | vacuolar protein sorting- |
| | | | | | associated protein 8 |
| | | | | | homolog isoform b |
| WASF3 | NM_001291965 | chr13 | 27131839 | 27263082 | wiskott-Aldrich |
| | | | | | syndrome protein family |
| | | | | | member 3 isoform 2 |
| WBSCR16 | NM_001281441 | chr7 | 74470621 | 74489717 | Williams-Beuren |
| | | | | | syndrome chromosomal |
| | | | | | region 16 protein |
| | | | | | isoform 3 |
| WDFY3 | NM_014991 | chr4 | 85590692 | 85887544 | WD repeat and FYVE |
| | | | | | domain-containing |
| | | | | | protein 3 |
| WDR17 | NM_181265 | chr4 | 176986984 | 177103979 | WD repeat-containing |
| | | | | | protein 17 isoform 2 |
| WISP1 | NM_080838 | chr8 | 134203281 | 134243932 | WNT1-inducible- |
| | | | | | signaling pathway |
| | | | | | protein 1 isoform 2 |
| | | | | | precursor |
| XRCC5 | NM_021141 | chr2 | 216974019 | 217071016 | X-ray repair cross- |
| | | | | | complementing protein |
| | | | | | 5 |
| YEATS2 | NM_018023 | chr3 | 183415605 | 183530413 | YEATS domain- |
| | | | | | containing protein 2 |
| ZBTB41 | NM_194314 | chr1 | 197122813 | 197169672 | zinc finger and BTB |
| | | | | | domain-containing |
| | | | | | protein 41 |
| ZDHHC20 | NM_153251 | chr13 | 21946709 | 22033508 | probable |
| | | | | | palmitoyltransferase |
| | | | | | ZDHHC20 isoform 1 |
| ZNF274 | NM_133502 | chr19 | 58694355 | 58724928 | neurotrophin receptor- |
| | | | | | interacting factor |
| | | | | | homolog isoform c |
| ZNF702P | NR_003578 | chr19 | 53471503 | 53496784 | |
| ZNF804B | NM_181646 | chr7 | 88388752 | 88966346 | zinc finger protein 804B |

TABLE 2
Genes with high expression and CN-invariant in the TCGA EOC
samples (see also Table 13 for the full gene annotation).

| Symbol | Probeset | Median expr | CV | Surv. Pvalue |
|---|---|---|---|---|
| PDIA3 | 208612_at | 10.62 | 0.04 | 0.20338 |
| PTPRF | 200636_s_at | 10.35 | 0.05 | 0.00022 |
| EIF5 | 208705_s_at | 10.13 | 0.04 | 0.02947 |
| PUM1 | 201166_s_at | 10.08 | 0.03 | 0.06748 |
| PTPRF | 200635_s_at | 9.88 | 0.05 | 0.00005 |
| NOTCH2 | 212377_s_at | 9.78 | 0.05 | 0.05414 |
| DYRK1A | 209033_s_at | 9.86 | 0.04 | 0.00317 |
| XRCC5 | 208642_s_at | 9.74 | 0.04 | 0.08567 |
| XRCC5 | 208643_s_at | 9.69 | 0.05 | 0.00579 |
| CLIC4 | 201560_at | 9.68 | 0.05 | 0.01722 |
| PUM1 | 201164_s_at | 9.57 | 0.04 | 0.33393 |
| COPA | 208684_at | 9.51 | 0.03 | 0.20760 |
| NECAP2 | 220731_s_at | 9.52 | 0.04 | 0.00636 |
| CUL3 | 201371_s_at | 9.5 | 0.03 | 0.04678 |
| SPATS2L | 222154_s_at | 9.53 | 0.06 | 0.02196 |
| DDAH1 | 209094_at | 9.5 | 0.07 | 0.17050 |
| DEGS1 | 209250_at | 9.25 | 0.07 | 0.02012 |
| BRE | 205550_s_at | 9.12 | 0.04 | 0.02472 |
| YEATS2 | 221203_s_at | 9.11 | 0.05 | 0.00149 |
| AMD1 | 201197_at | 9.12 | 0.04 | 0.02027 |
| DBT | 205370_x_at | 9.1 | 0.04 | 0.04285 |
| MTA1 | 211783_s_at | 9.06 | 0.06 | 0.03996 |
| PUM1 | 201165_s_at | 9.08 | 0.04 | 0.03005 |
| FHL2 | 202949_s_at | 9.03 | 0.09 | 0.00859 |
| NOTCH2 | 202443_x_at | 9.02 | 0.05 | 0.01107 |
| GPBP1L1 | 217877_s_at | 8.98 | 0.03 | 0.00688 |
| CP | 204846_at | 9.09 | 0.14 | 0.12168 |
| SERTAD2 | 202657_s_at | 8.79 | 0.05 | 0.03068 |
| EHBP1 | 212653_s_at | 8.64 | 0.04 | 0.01322 |
| GBE1 | 203282_at | 8.65 | 0.05 | 0.17699 |
| FAT1 | 201579_at | 8.77 | 0.1 | 0.06658 |
| AUTS2 | 212599_at | 8.6 | 0.07 | 0.13549 |
| EIF5 | 208706_s_at | 8.59 | 0.05 | 0.17068 |
| PRUNE | 209586_s_at | 8.45 | 0.05 | 0.13525 |
| RAI2 | 219440_at | 8.49 | 0.09 | 0.10687 |
| EIF5 | 208708_x_at | 8.44 | 0.06 | 0.00692 |
| PTPRF | 200637_s_at | 8.37 | 0.06 | 0.01045 |
| SERTAD2 | 202656_s_at | 8.35 | 0.05 | 0.04363 |
| FHL1 | 201540_at | 8.27 | 0.12 | 0.09021 |
| TBL1X | 213400_s_at | 8.38 | 0.09 | 0.04973 |
| NUP133 | 202184_s_at | 8.36 | 0.04 | 0.00319 |
| NT5C2 | 209155_s_at | 8.28 | 0.05 | 0.32412 |
| TGFBR3 | 204731_at | 8.15 | 0.08 | 0.01399 |
| VPS8 | 209553_at | 8.17 | 0.05 | 0.02758 |
| PARN | 203905_at | 8.14 | 0.05 | 0.07753 |
| DAPK1 | 203139_at | 8.1 | 0.07 | 0.07083 |
| ERBB4 | 214053_at | 8.19 | 0.13 | 0.08732 |
| TIAM1 | 213135_at | 8.1 | 0.07 | 0.12098 |
| SCHIP1 | 204030_s_at | 8.07 | 0.09 | 0.08119 |
| MTR | 203774_at | 8.06 | 0.06 | 0.12443 |
| SMYD3 | 218788_s_at | 8.11 | 0.06 | 0.02778 |
| ZNF274 | 204937_s_at | 8.05 | 0.05 | 0.05063 |
| DEGS1 | 207431_s_at | 8.03 | 0.07 | 0.00519 |
| BRE | 212645_x_at | 8.01 | 0.04 | 0.07055 |
| BRE | 211566_x_at | 8.01 | 0.04 | 0.11351 |
| KIAA0430 | 202386_s_at | 8.01 | 0.04 | 0.00140 |
| TTF1 | 204771_s_at | 7.99 | 0.04 | 0.27136 |
| ENPP2 | 209392_at | 7.93 | 0.09 | 0.00721 |
| AGAP1 | 204066_s_at | 7.99 | 0.06 | 0.04297 |
| PRKCZ | 202178_at | 7.95 | 0.06 | 0.11192 |
| FAHD2A | 222056_s_at | 7.89 | 0.05 | 0.03631 |
| AMD1 | 201196_s_at | 7.85 | 0.05 | 0.07653 |
| NOTCH2 | 210756_s_at | 7.81 | 0.04 | 0.12557 |
| MORC3 | 213000_at | 7.81 | 0.04 | 0.02729 |
| CHST15 | 203066_at | 7.82 | 0.1 | 0.00896 |
| RNF144A | 204040_at | 7.75 | 0.08 | 0.05543 |
| ASCC3 | 212815_at | 7.75 | 0.05 | 0.10970 |
| ACYP2 | 206833_s_at | 7.69 | 0.07 | 0.00031 |
| EIF5 | 208290_s_at | 7.65 | 0.06 | 0.01586 |
| CLMN | 221042_s_at | 7.63 | 0.06 | 0.30167 |
| FAHD2A | 218504_at | 7.59 | 0.05 | 0.15978 |
| LEF1 | 221558_s_at | 7.49 | 0.12 | 0.01963 |
| CLASP1 | 212752_at | 7.57 | 0.04 | 0.20654 |
| WASF3 | 204042_at | 7.6 | 0.09 | 0.02224 |
| TSPAN9 | 220968_s_at | 7.58 | 0.05 | 0.00037 |
| TBL1X | 201867_s_at | 7.54 | 0.07 | 0.02455 |
| CLIC4 | 221881_s_at | 7.56 | 0.06 | 0.02110 |
| PRUNE | 210988_s_at | 7.46 | 0.04 | 0.23481 |
| SLC15A2 | 205316_at | 7.35 | 0.1 | 0.01251 |
| WDFY3 | 212602_at | 7.44 | 0.05 | 0.12013 |
| RAB11FIP1 | 219681_s_at | 7.33 | 0.08 | 0.07390 |
| WBSCR16 | 221247_s_at | 7.39 | 0.04 | 0.03208 |
| EHBP1 | 212650_at | 7.37 | 0.03 | 0.01359 |
| NMD3 | 218036_x_at | 7.35 | 0.04 | 0.09489 |
| POU2F1 | 206789_s_at | 7.38 | 0.04 | 0.06434 |
| BMPR2 | 210214_s_at | 7.33 | 0.05 | 0.00025 |
| ATXN7 | 204516_at | 7.33 | 0.05 | 0.02880 |
| PTPRF | 215066_at | 7.26 | 0.03 | 0.04876 |
| FHIT | 206492_at | 7.2 | 0.07 | 0.19039 |
| EPHB2 | 211165_x_at | 7.18 | 0.06 | 0.01610 |
| FCGR2A | 203561_at | 7.18 | 0.1 | 0.00242 |
| ARHGAP10 | 219431_at | 7.19 | 0.04 | 0.19969 |
| PHTF1 | 210191_s_at | 7.17 | 0.04 | 0.00273 |
| ENPP2 | 210839_s_at | 7.08 | 0.07 | 0.03070 |
| FHL1 | 210299_s_at | 7.01 | 0.12 | 0.06449 |
| IL15 | 205992_s_at | 7.13 | 0.12 | 0.07816 |
| H6PD | 221892_at | 7.14 | 0.05 | 0.01491 |
| WDFY3 | 212606_at | 7.14 | 0.04 | 0.04054 |
| NLGN4X | 221933_at | 6.97 | 0.1 | 0.02676 |
| ABHD5 | 218739_at | 7.13 | 0.04 | 0.06548 |
| CLIC4 | 201559_s_at | 7.13 | 0.05 | 0.00946 |
| CLMN | 213839_at | 7.08 | 0.07 | 0.07973 |
| CHL1 | 204591_at | 6.99 | 0.15 | 0.07302 |
| EPHB2 | 209588_at | 7.09 | 0.05 | 0.15543 |
| MAN1A1 | 221760_at | 7.12 | 0.11 | 0.05231 |
| BMPR2 | 209920_at | 7.11 | 0.05 | 0.00521 |
| EPHB2 | 210651_s_at | 7.08 | 0.03 | 0.03742 |
| FGF12 | 214589_at | 7.1 | 0.02 | 0.07807 |
| FGGY | 219718_at | 7.04 | 0.05 | 0.04990 |
| TLE4 | 204872_at | 7.01 | 0.09 | 0.14776 |
| FUT9 | 216185_at | 7.07 | 0.02 | 0.02171 |
| EPHB2 | 209589_s_at | 7.01 | 0.06 | 0.06130 |
| ASAP1 | 221039_s_at | 7.01 | 0.05 | 0.00590 |
| IL5RA | 210744_s_at | 7.05 | 0.02 | 0.03824 |
| EFHC2 | 220591_s_at | 6.94 | 0.08 | 0.02003 |
| TTF1 | 204772_s_at | 7.03 | 0.03 | 0.00623 |
| ATF7IP2 | 219870_at | 7.03 | 0.04 | 0.09257 |
| ANK2 | 202920_at | 6.88 | 0.11 | 0.13741 |
| MFAP3L | 210493_s_at | 7.02 | 0.02 | 0.18480 |
| GOLIM4 | 204324_s_at | 7 | 0.05 | 0.19382 |
| EHD3 | 218935_at | 7 | 0.05 | 0.15127 |
| DAB1 | 220611_at | 7.01 | 0.02 | 0.01393 |
| DBT | 205369_x_at | 7 | 0.04 | 0.03095 |
| FHL1 | 214505_s_at | 6.86 | 0.09 | 0.01801 |
| TGFBRAP1 | 205210_at | 6.95 | 0.03 | 0.00127 |
| PHTF1 | 205702_at | 6.91 | 0.04 | 0.00146 |
| TIAM1 | 206409_at | 6.9 | 0.03 | 0.28210 |
| LDB2 | 206481_s_at | 6.86 | 0.05 | 0.07078 |
| ABHD5 | 213935_at | 6.89 | 0.03 | 0.04094 |
| CACNA2D1 | 207050_at | 6.9 | 0.02 | 0.29669 |
| LYST | 210943_s_at | 6.86 | 0.04 | 0.14418 |
| RAD52 | 205647_at | 6.87 | 0.03 | 0.02273 |
| CUL3 | 201370_s_at | 6.87 | 0.07 | 0.03293 |
| LEF1 | 210948_s_at | 6.77 | 0.09 | 0.07087 |
| HHAT | 219687_at | 6.84 | 0.06 | 0.00428 |
| EPB41 | 207793_s_at | 6.87 | 0.02 | 0.01335 |
| ATAD2B | 213387_at | 6.83 | 0.03 | 0.01759 |
| DBT | 205371_s_at | 6.82 | 0.04 | 0.06851 |
| GTF2F2 | 209595_at | 6.8 | 0.03 | 0.01296 |
| ESRRG | 207981_s_at | 6.73 | 0.07 | 0.09335 |
| FHL1 | 210298_x_at | 6.67 | 0.09 | 0.00971 |
| KIT | 205051_s_at | 6.73 | 0.06 | 0.00802 |
| DNM3 | 209839_at | 6.72 | 0.05 | 0.01017 |
| PCDH7 | 205535_s_at | 6.78 | 0.03 | 0.01285 |
| NEIL3 | 219502_at | 6.76 | 0.03 | 0.09424 |
| C1orf21 | 221272_s_at | 6.75 | 0.03 | 0.02970 |
| MFAP3L | 205442_at | 6.68 | 0.06 | 0.15633 |
| GLI2 | 208057_s_at | 6.76 | 0.04 | 0.03577 |
| PLEKHA2 | 217677_at | 6.74 | 0.03 | 0.04937 |
| FAM49A | 208092_s_at | 6.69 | 0.05 | 0.01330 |
| COPA | 214336_s_at | 6.75 | 0.04 | 0.00146 |
| DEPDC1 | 220295_x_at | 6.7 | 0.07 | 0.05928 |
| WDFY3 | 212598_at | 6.73 | 0.02 | 0.00706 |
| TBL1X | 201868_s_at | 6.69 | 0.05 | 0.02552 |
| ERBB4 | 206794_at | 6.67 | 0.04 | 0.05339 |
| HYAL3 | 211728_s_at | 6.67 | 0.05 | 0.05147 |
| BTNL8 | 220421_at | 6.68 | 0.04 | 0.04656 |
| HRG | 31835_at | 6.69 | 0.02 | 0.02679 |
| TBL1X | 201869_s_at | 6.66 | 0.05 | 0.05697 |
| KCNAB1 | 210079_x_at | 6.69 | 0.02 | 0.02286 |
| LYST | 203518_at | 6.66 | 0.04 | 0.00863 |
| PDE2A | 204134_at | 6.64 | 0.03 | 0.01786 |
| NOTCH2 | 202445_s_at | 6.63 | 0.04 | 0.00017 |
| SP4 | 206663_at | 6.66 | 0.02 | 0.06132 |
| TNIK | 213107_at | 6.61 | 0.05 | 0.00333 |
| SLC15A2 | 205317_s_at | 6.56 | 0.05 | 0.02679 |
| ESRRG | 209966_x_at | 6.57 | 0.07 | 0.00368 |
| LAMC3 | 219407_s_at | 6.58 | 0.06 | 0.02266 |
| PCDH7 | 210273_at | 6.58 | 0.06 | 0.03610 |
| MTA1 | 202247_s_at | 6.64 | 0.03 | 0.05778 |
| DAPK1 | 211214_s_at | 6.63 | 0.02 | 0.07588 |
| AFF3 | 205735_s_at | 6.64 | 0.02 | 0.06791 |
| HS3ST1 | 213991_s_at | 6.62 | 0.03 | 0.08849 |
| PHTF1 | 215285_s_at | 6.6 | 0.04 | 0.00014 |
| IL15 | 217371_s_at | 6.55 | 0.07 | 0.00521 |
| HS3ST1 | 205466_s_at | 6.58 | 0.07 | 0.06365 |
| PCDH7 | 205534_at | 6.47 | 0.1 | 0.04277 |
| LPHN3 | 209867_s_at | 6.56 | 0.04 | 0.00607 |
| PCOLCE2 | 219295_s_at | 6.53 | 0.05 | 0.03009 |
| FHL1 | 201539_s_at | 6.48 | 0.07 | 0.00691 |
| ABHD5 | 213805_at | 6.56 | 0.02 | 0.03415 |
| CAMTA1 | 213268_at | 6.53 | 0.05 | 0.04646 |
| CASQ2 | 207317_s_at | 6.53 | 0.03 | 0.16039 |
| RAD52 | 211904_x_at | 6.57 | 0.03 | 0.13310 |
| ATXN7 | 209964_s_at | 6.55 | 0.02 | 0.06355 |
| SLC4A4 | 210739_x_at | 6.55 | 0.02 | 0.04069 |
| GRM8 | 216256_at | 6.55 | 0.01 | 0.04053 |
| THRAP3 | 217847_s_at | 6.55 | 0.02 | 0.00935 |
| HTR4 | 207578_s_at | 6.54 | 0.01 | 0.21199 |
| MAN1A1 | 208116_s_at | 6.52 | 0.04 | 0.04868 |
| TRPM8 | 220226_at | 6.53 | 0.02 | 0.12609 |
| PRKCE | 206248_at | 6.52 | 0.02 | 0.03066 |
| TBL1X | 213401_s_at | 6.51 | 0.03 | 0.12794 |
| EIF5 | 208707_at | 6.49 | 0.03 | 0.02177 |
| TNIK | 213109_at | 6.42 | 0.07 | 0.00566 |
| PRUNE | 209599_s_at | 6.51 | 0.03 | 0.10137 |
| TLE4 | 214688_at | 6.48 | 0.04 | 0.21103 |
| CUL3 | 201372_s_at | 6.51 | 0.03 | 0.07651 |
| DYRK1A | 211541_s_at | 6.5 | 0.03 | 0.02780 |
| BATF3 | 220358_at | 6.48 | 0.02 | 0.11090 |
| NRP2 | 214632_at | 6.47 | 0.04 | 0.13341 |
| SLC4A4 | 203908_at | 6.43 | 0.06 | 0.10032 |
| SLC12A6 | 220740_s_at | 6.5 | 0.02 | 0.09519 |
| FGF12 | 207501_s_at | 6.44 | 0.03 | 0.07473 |
| PTGS2 | 204748_at | 6.35 | 0.08 | 0.10158 |
| GLI2 | 207034_s_at | 6.43 | 0.03 | 0.00107 |
| KCNAB1 | 210078_s_at | 6.44 | 0.04 | 0.16319 |
| TSPAN9 | 205665_at | 6.42 | 0.03 | 0.05611 |
| ZNF702P | 206557_at | 6.41 | 0.04 | 0.05041 |
| NRP2 | 210841_s_at | 6.42 | 0.02 | 0.24581 |
| ANK2 | 202921_s_at | 6.41 | 0.02 | 0.13182 |
| CACNB2 | 207776_s_at | 6.43 | 0.01 | 0.28364 |
| GAP43 | 216963_s_at | 6.42 | 0.02 | 0.00607 |
| PTPRZ1 | 204469_at | 6.41 | 0.04 | 0.00006 |
| RAD52 | 210630_s_at | 6.39 | 0.03 | 0.00192 |
| FAM49A | 209683_at | 6.38 | 0.04 | 0.00367 |
| TNIK | 211828_s_at | 6.34 | 0.05 | 0.12912 |
| IL5RA | 211516_at | 6.38 | 0.03 | 0.03421 |
| CACNB2 | 213714_at | 6.38 | 0.02 | 0.00153 |
| LPHN3 | 209866_s_at | 6.25 | 0.07 | 0.00313 |
| TEC | 206301_at | 6.37 | 0.02 | 0.01093 |
| GAP43 | 204471_at | 6.35 | 0.03 | 0.03357 |
| PRDM5 | 220792_at | 6.37 | 0.02 | 0.05073 |
| KCNAB1 | 208213_s_at | 6.37 | 0.01 | 0.14705 |
| ARSE | 205894_at | 6.33 | 0.03 | 0.08378 |
| CCDC88A | 219387_at | 6.31 | 0.05 | 0.26252 |
| IL5RA | 207902_at | 6.34 | 0.01 | 0.04565 |
| ANK2 | 216195_at | 6.34 | 0.02 | 0.09666 |
| TLE4 | 216997_x_at | 6.34 | 0.02 | 0.02096 |
| ERC2 | 213938_at | 6.31 | 0.03 | 0.14336 |
| HS3ST1 | 205465_x_at | 6.34 | 0.02 | 0.04735 |
| SLC4A4 | 211494_s_at | 6.31 | 0.02 | 0.04845 |
| CACNB2 | 215365_at | 6.32 | 0.01 | 0.04082 |
| COPA | 214337_at | 6.32 | 0.01 | 0.11916 |
| PDZK1 | 205380_at | 6.22 | 0.06 | 0.04122 |
| CCDC88A | 221078_s_at | 6.31 | 0.02 | 0.06450 |
| HTR4 | 216939_s_at | 6.31 | 0.02 | 0.00770 |
| HRG | 206226_at | 6.3 | 0.02 | 0.01240 |
| NRP2 | 211844_s_at | 6.29 | 0.03 | 0.00660 |
| WISP1 | 206796_at | 6.25 | 0.04 | 0.00666 |
| LYST | 215415_s_at | 6.29 | 0.01 | 0.00385 |
| H6PD | 206933_s_at | 6.28 | 0.01 | 0.00046 |
| NTNG1 | 206713_at | 6.28 | 0.01 | 0.12339 |
| WISP1 | 211312_s_at | 6.28 | 0.01 | 0.01658 |
| NRXN1 | 209914_s_at | 6.28 | 0.01 | 0.14478 |
| MCTP1 | 220122_at | 6.23 | 0.04 | 0.04156 |
| IL5RA | 211517_s_at | 6.26 | 0.02 | 0.29333 |
| MFAP3L | 210843_s_at | 6.26 | 0.02 | 0.01571 |
| PRDM16 | 220928_s_at | 6.26 | 0.02 | 0.00062 |
| LEF1 | 221557_s_at | 6.26 | 0.01 | 0.11284 |
| NRXN1 | 216096_s_at | 6.24 | 0.03 | 0.00120 |
| SLC4A4 | 210738_s_at | 6.24 | 0.03 | 0.15578 |
| HTR4 | 207577_at | 6.26 | 0.01 | 0.26027 |
| TRIM48 | 220534_at | 6.25 | 0.02 | 0.11769 |
| DBT | 211196_at | 6.25 | 0.01 | 0.02950 |
| GRM8 | 216992_s_at | 6.25 | 0.02 | 0.00285 |
| SPATS2L | 215617_at | 6.23 | 0.03 | 0.02000 |
| ABCB4 | 207819_s_at | 6.24 | 0.02 | 0.01195 |
| AFF3 | 205734_s_at | 6.24 | 0.01 | 0.08057 |
| NRP2 | 210842_at | 6.22 | 0.02 | 0.17198 |
| KCNAB1 | 210471_s_at | 6.2 | 0.02 | 0.01435 |
| MFAP3L | 210492_at | 6.19 | 0.02 | 0.01254 |
| EFHC2 | 220523_at | 6.2 | 0.01 | 0.01661 |
| EPB41 | 214530_x_at | 6.2 | 0.01 | 0.00585 |
| GRM8 | 216255_s_at | 6.2 | 0.01 | 0.02002 |
| DYRK1A | 211079_s_at | 6.19 | 0.01 | 0.11899 |
| FUT9 | 207696_at | 6.14 | 0.01 | 0.05224 |
| FUT9 | 214046_at | 6.13 | 0.03 | 0.06542 |
| LRCH1 | 214936_at | 6.13 | 0.02 | 0.07138 |
| NRXN1 | 209915_s_at | 6.12 | 0.01 | 0.16486 |
| LRP1B | 219643_at | 6.06 | 0.04 | 0.02452 |
| SNTG2 | 220487_at | 6.08 | 0.01 | 0.12133 |
| PDE6C | 211093_at | 6.07 | 0.01 | 0.03750 |
| PCDH7 | 210941_at | 6.03 | 0.03 | 0.04561 |
| CASC5 | 220247_at | 6 | 0.01 | 0.11084 |
| DPPA4 | 219651_at | 5.95 | 0.04 | 0.00008 |

Median expr = median log expression value across the samples; CV = coefficient of variation of the log expression values; Surv. P value = survival p-value.

TABLE 3
Genes with high expression in GSE9899 and CN-invariant in TCGA
EOC samples (see also Table 14 for the full gene annotation).

| Symbol | Probeset | Median expr | CV | Surv. Pvalue |
|---|---|---|---|---|
| DBT | 205370_x_at | 12.25 | 0.02 | 0.02040 |
| NOTCH2 | 202443_x_at | 11.46 | 0.04 | 0.17253 |
| PDIA3 | 208612_at | 11.24 | 0.04 | 0.02038 |
| PUM1 | 201166_s_at | 11.21 | 0.03 | 0.03512 |
| XRCC5 | 208642_s_at | 11.09 | 0.03 | 0.22739 |
| PTPRF | 200636_s_at | 11.06 | 0.05 | 0.24272 |
| NOTCH2 | 212377_s_at | 10.86 | 0.04 | 0.02659 |
| CLIC4 | 201560_at | 10.77 | 0.05 | 0.00009 |
| SPATS2L | 222154_s_at | 10.68 | 0.06 | 0.01236 |
| COPA | 208684_at | 10.66 | 0.03 | 0.00455 |
| EIF5 | 208705_s_at | 10.65 | 0.04 | 0.06987 |
| PUM1 | 201164_s_at | 10.64 | 0.03 | 0.02840 |
| XRCC5 | 208643_s_at | 10.62 | 0.04 | 0.06877 |
| CUL3 | 201371_s_at | 10.46 | 0.03 | 0.03970 |
| CP | 204846_at | 10.36 | 0.13 | 0.02147 |
| DYRK1A | 209033_s_at | 10.34 | 0.03 | 0.12664 |
| FHL2 | 202949_s_at | 10.25 | 0.08 | 0.11226 |
| PUM1 | 201165_s_at | 10.17 | 0.04 | 0.07656 |
| AUTS2 | 212599_at | 9.99 | 0.06 | 0.06148 |
| NT5C2 | 209155_s_at | 9.95 | 0.04 | 0.00538 |
| EIF5 | 208706_s_at | 9.93 | 0.04 | 0.06033 |
| DDAH1 | 209094_at | 9.92 | 0.06 | 0.01562 |
| DEGS1 | 209250_at | 9.88 | 0.06 | 0.00232 |
| PTPRF | 200635_s_at | 9.85 | 0.06 | 0.06567 |
| AMD1 | 201197_at | 9.8 | 0.04 | 0.05652 |
| GPBP1L1 | 217877_s_at | 9.76 | 0.03 | 0.04268 |
| YEATS2 | 221203_s_at | 9.69 | 0.05 | 0.00233 |
| GLI2 | 208057_s_at | 9.64 | 0.05 | 0.30579 |
| FAT1 | 201579_at | 9.58 | 0.1 | 0.00624 |
| FHL1 | 201540_at | 9.58 | 0.1 | 0.03419 |
| PARN | 203905_at | 9.55 | 0.03 | 0.27358 |
| NUP133 | 202184_s_at | 9.52 | 0.04 | 0.18819 |
| NECAP2 | 220731_s_at | 9.51 | 0.04 | 0.01493 |
| SERTAD2 | 202657_s_at | 9.49 | 0.05 | 0.00899 |
| ATXN7 | 204516_at | 9.47 | 0.04 | 0.01148 |
| CHST15 | 203066_at | 9.47 | 0.08 | 0.00870 |
| EIF5 | 208708_x_at | 9.46 | 0.04 | 0.04619 |
| MORC3 | 213000_at | 9.46 | 0.04 | 0.01305 |
| GBE1 | 203282_at | 9.45 | 0.05 | 0.03451 |
| BRE | 205550_s_at | 9.34 | 0.04 | 0.18017 |
| LEF1 | 221558_s_at | 9.32 | 0.1 | 0.06360 |
| SERTAD2 | 202656_s_at | 9.3 | 0.05 | 0.01998 |
| RAI2 | 219440_at | 9.24 | 0.09 | 0.00090 |
| MTA1 | 211783_s_at | 9.21 | 0.05 | 0.06242 |
| DAPK1 | 203139_at | 9.17 | 0.06 | 0.11341 |
| PRUNE | 209586_s_at | 9.17 | 0.05 | 0.00825 |
| DEGS1 | 207431_s_at | 9.17 | 0.06 | 0.01518 |
| RNF144A | 204040_at | 9.08 | 0.07 | 0.04822 |
| PTPRF | 215066_at | 9.04 | 0.04 | 0.05418 |
| SMYD3 | 218788_s_at | 9.04 | 0.06 | 0.00233 |
| EHBP1 | 212653_s_at | 9.03 | 0.04 | 0.00489 |
| TBL1X | 213400_s_at | 9.03 | 0.06 | 0.06571 |
| MAN1A1 | 221760_at | 9.02 | 0.1 | 0.04635 |
| NOTCH2 | 210756_s_at | 9.01 | 0.05 | 0.03153 |
| PTPRF | 200637_s_at | 9.01 | 0.07 | 0.09062 |
| WBSCR16 | 221247_s_at | 9 | 0.03 | 0.00512 |
| tabular VPS8 | 209553_at | 8.96 | 0.04 | 0.01131 |
| BRE | 212645_x_at | 8.95 | 0.03 | 0.29607 |
| KIAA0430 | 202386_s_at | 8.89 | 0.04 | 0.08524 |
| BRE | 211566_x_at | 8.89 | 0.04 | 0.22934 |
| TTF1 | 204771_s_at | 8.86 | 0.05 | 0.04547 |
| MTR | 203774_at | 8.82 | 0.05 | 0.13164 |
| NMD3 | 218036_x_at | 8.81 | 0.04 | 0.17399 |
| CUL3 | 201370_s_at | 8.81 | 0.05 | 0.09902 |
| EIF5 | 208290_s_at | 8.81 | 0.05 | 0.05245 |
| TSPAN9 | 220968_s_at | 8.79 | 0.04 | 0.00043 |
| FCGR2A | 203561_at | 8.76 | 0.09 | 0.15164 |
| TIAM1 | 213135_at | 8.75 | 0.07 | 0.02124 |
| AGAP1 | 204066_s_at | 8.74 | 0.06 | 0.01199 |
| ENPP2 | 209392_at | 8.73 | 0.09 | 0.01476 |
| AMD1 | 201196_s_at | 8.68 | 0.04 | 0.06565 |
| FAHD2A | 222056_s_at | 8.68 | 0.05 | 0.08837 |
| ZNF274 | 204937_s_at | 8.67 | 0.05 | 0.14136 |
| ERBB4 | 214053_at | 8.6 | 0.14 | 0.01026 |
| FAHD2A | 218504_at | 8.59 | 0.03 | 0.01900 |
| ASCC3 | 212815_at | 8.56 | 0.05 | 0.18424 |
| ATXN7 | 209964_s_at | 8.54 | 0.05 | 0.01107 |
| ASAP1 | 221039_s_at | 8.53 | 0.05 | 0.11827 |
| CLASP1 | 212752_at | 8.47 | 0.03 | 0.00053 |
| HRG | 31835_at | 8.43 | 0.03 | 0.07209 |
| CLMN | 213839_at | 8.42 | 0.06 | 0.00381 |
| TLE4 | 204872_at | 8.29 | 0.1 | 0.05946 |
| H6PD | 221892_at | 8.28 | 0.05 | 0.01582 |
| PRKCZ | 202178_at | 8.28 | 0.05 | 0.09564 |
| SCHIP1 | 204030_s_at | 8.24 | 0.08 | 0.00021 |
| EPHB2 | 209588_at | 8.21 | 0.03 | 0.00274 |
| WDFY3 | 212606_at | 8.21 | 0.04 | 0.00012 |
| TIAM1 | 206409_at | 8.18 | 0.04 | 0.07169 |
| PRUNE | 210988_s_at | 8.17 | 0.04 | 0.02233 |
| CLMN | 221042_s_at | 8.15 | 0.06 | 0.04387 |
| POU2F1 | 206789_s_at | 8.13 | 0.03 | 0.03589 |
| TGFBR3 | 204731_at | 8.12 | 0.09 | 0.02006 |
| WASF3 | 204042_at | 8.1 | 0.09 | 0.00186 |
| ENPP2 | 210839_s_at | 8.09 | 0.08 | 0.01530 |
| EPHB2 | 210651_s_at | 8.06 | 0.03 | 0.00118 |
| CLIC4 | 201559_s_at | 8.06 | 0.07 | 0.10860 |
| RAB11FIP1 | 219681_s_at | 8.03 | 0.09 | 0.08002 |
| FHL1 | 214505_s_at | 8.02 | 0.06 | 0.00468 |
| CHL1 | 204591_at | 8.01 | 0.15 | 0.07569 |
| WDFY3 | 212602_at | 8 | 0.04 | 0.31880 |
| CLIC4 | 221881_s_at | 8 | 0.06 | 0.04131 |
| TBL1X | 201869_s_at | 7.96 | 0.05 | 0.03666 |
| EPHB2 | 209589_s_at | 7.93 | 0.06 | 0.00015 |
| AXF7IP2 | 219870_at | 7.93 | 0.05 | 0.06342 |
| ACYP2 | 206833_s_at | 7.93 | 0.05 | 0.12086 |
| HS3ST1 | 205465_x_at | 7.91 | 0.03 | 0.00249 |
| CACNA2D1 | 207050_at | 7.9 | 0.03 | 0.00314 |
| FHL1 | 210299_s_at | 7.89 | 0.1 | 0.01261 |
| PHXF1 | 210191_s_at | 7.86 | 0.04 | 0.02938 |
| HXR4 | 207578_s_at | 7.85 | 0.02 | 0.00334 |
| PCDH7 | 210273_at | 7.81 | 0.06 | 0.03321 |
| KCNAB1 | 208213_s_at | 7.81 | 0.04 | 0.21222 |
| PHXF1 | 205702_at | 7.79 | 0.04 | 0.07287 |
| TBL1X | 201867_s_at | 7.79 | 0.1 | 0.17749 |
| EHD3 | 218935_at | 7.78 | 0.05 | 0.03854 |
| GTF2F2 | 209595_at | 7.78 | 0.04 | 0.04245 |
| LAMC3 | 219407_s_at | 7.78 | 0.03 | 0.00270 |
| EHBP1 | 212650_at | 7.75 | 0.04 | 0.11393 |
| TTF1 | 204772_s_at | 7.75 | 0.04 | 0.01049 |
| GAP43 | 216963_s_at | 7.74 | 0.03 | 0.00619 |
| LEF1 | 221557_s_at | 7.72 | 0.02 | 0.00335 |
| SLC15A2 | 205316_at | 7.69 | 0.1 | 0.08613 |
| RAD52 | 205647_at | 7.68 | 0.06 | 0.07622 |
| BMPR2 | 209920_at | 7.68 | 0.04 | 0.05334 |
| ATAD2B | 213387_at | 7.66 | 0.05 | 0.00089 |
| BMPR2 | 210214_s_at | 7.66 | 0.05 | 0.05113 |
| COPA | 214336_s_at | 7.64 | 0.07 | 0.02071 |
| FGGY | 219718_at | 7.64 | 0.04 | 0.06761 |
| LYST | 203518_at | 7.63 | 0.05 | 0.01240 |
| DBT | 205369_x_at | 7.62 | 0.04 | 0.01505 |
| LDB2 | 206481_s_at | 7.62 | 0.07 | 0.00034 |
| NEIL3 | 219502_at | 7.62 | 0.03 | 0.24524 |
| IL15 | 205992_s_at | 7.62 | 0.1 | 0.08336 |
| NRP2 | 210841_s_at | 7.6 | 0.03 | 0.00028 |
| PCDH7 | 205535_s_at | 7.59 | 0.04 | 0.10384 |
| CACNB2 | 215365_at | 7.58 | 0.04 | 0.00327 |
| C1orf21 | 221272_s_at | 7.57 | 0.04 | 0.04363 |
| NRP2 | 214632_at | 7.56 | 0.03 | 0.04684 |
| EPHB2 | 211165_x_at | 7.56 | 0.04 | 0.00019 |
| FHL1 | 210298_x_at | 7.55 | 0.08 | 0.05729 |
| EIF5 | 208707_at | 7.55 | 0.03 | 0.06981 |
| LYST | 210943_s_at | 7.54 | 0.04 | 0.16516 |
| CASQ2 | 207317_s_at | 7.54 | 0.04 | 0.06762 |
| GOLIM4 | 204324_s_at | 7.53 | 0.05 | 0.06101 |
| ANK2 | 202920_at | 7.53 | 0.11 | 0.21165 |
| ABHD5 | 218739_at | 7.52 | 0.04 | 0.00029 |
| BATF3 | 220358_at | 7.5 | 0.02 | 0.09950 |
| KIT | 205051_s_at | 7.48 | 0.06 | 0.12776 |
| TGFBRAP1 | 205210_at | 7.47 | 0.03 | 0.00931 |
| PHTF1 | 215285_s_at | 7.45 | 0.05 | 0.00664 |
| FHL1 | 201539_s_at | 7.44 | 0.07 | 0.08433 |
| ESRRG | 207981_s_at | 7.4 | 0.09 | 0.02416 |
| FHIT | 206492_at | 7.39 | 0.05 | 0.04854 |
| TRPM8 | 220226_at | 7.39 | 0.02 | 0.01284 |
| NLGN4X | 221933_at | 7.38 | 0.12 | 0.05823 |
| TSPAN9 | 205665_at | 7.37 | 0.03 | 0.06193 |
| SLC15A2 | 205317_s_at | 7.37 | 0.05 | 0.01063 |
| FAM49A | 208092_s_at | 7.37 | 0.04 | 0.05475 |
| IL5RA | 210744_s_at | 7.36 | 0.02 | 0.31680 |
| THRAP3 | 217847_s_at | 7.34 | 0.03 | 0.04736 |
| PDE2A | 204134_at | 7.34 | 0.03 | 0.04255 |
| MTA1 | 202247_s_at | 7.33 | 0.03 | 0.03778 |
| DBT | 205371_s_at | 7.32 | 0.05 | 0.00536 |
| PRUNE | 209599_s_at | 7.32 | 0.04 | 0.19033 |
| PLEKHA2 | 217677_at | 7.3 | 0.03 | 0.05817 |
| WDFY3 | 212598_at | 7.29 | 0.03 | 0.05518 |
| COPA | 214337_at | 7.29 | 0.04 | 0.04946 |
| PCDH7 | 205534_at | 7.28 | 0.11 | 0.12498 |
| H6PD | 206933_s_at | 7.28 | 0.03 | 0.00634 |
| CAMTA1 | 213268_at | 7.27 | 0.07 | 0.00659 |
| ARHGAP10 | 219431_at | 7.26 | 0.04 | 0.01507 |
| BTNL8 | 220421_at | 7.26 | 0.02 | 0.00210 |
| TLE4 | 214688_at | 7.25 | 0.06 | 0.03273 |
| SLC4A4 | 210739_x_at | 7.25 | 0.02 | 0.03900 |
| IL15 | 217371_s_at | 7.23 | 0.06 | 0.10346 |
| HHAT | 219687_at | 7.22 | 0.04 | 0.01657 |
| ABHD5 | 213805_at | 7.22 | 0.05 | 0.01621 |
| TBL1X | 201868_s_at | 7.22 | 0.03 | 0.03174 |
| PRDM16 | 220928_s_at | 7.21 | 0.04 | 0.24362 |
| NOTCH2 | 202445_s_at | 7.2 | 0.03 | 0.19662 |
| PRDM5 | 220792_at | 7.2 | 0.02 | 0.00483 |
| HTR4 | 216939_s_at | 7.2 | 0.03 | 0.00420 |
| ABHD5 | 213935_at | 7.19 | 0.04 | 0.02363 |
| LYST | 215415_s_at | 7.19 | 0.02 | 0.03630 |
| DAPK1 | 211214_s_at | 7.19 | 0.03 | 0.00220 |
| TNIK | 213107_at | 7.18 | 0.08 | 0.00314 |
| FGF12 | 214589_at | 7.17 | 0.03 | 0.01345 |
| GRM8 | 216256_at | 7.17 | 0.02 | 0.26278 |
| MAN1A1 | 208116_s_at | 7.15 | 0.08 | 0.10024 |
| HRG | 206226_at | 7.15 | 0.02 | 0.02982 |
| TNIK | 211828_s_at | 7.13 | 0.08 | 0.00719 |
| DYRK1A | 211541_s_at | 7.13 | 0.02 | 0.00907 |
| CCDC88A | 221078_s_at | 7.13 | 0.04 | 0.00820 |
| EFHC2 | 220591_s_at | 7.13 | 0.08 | 0.00176 |
| CACNB2 | 207776_s_at | 7.11 | 0.02 | 0.07419 |
| FAM49A | 209683_at | 7.09 | 0.05 | 0.12475 |
| DEPDC1 | 220295_x_at | 7.08 | 0.07 | 0.03224 |
| ZNF702P | 206557_at | 7.08 | 0.05 | 0.09070 |
| LPHN3 | 209867_s_at | 7.05 | 0.05 | 0.07323 |
| MFAP3L | 210493_s_at | 7.05 | 0.02 | 0.00583 |
| ANK2 | 202921_s_at | 7.04 | 0.03 | 0.01616 |
| SLC4A4 | 203908_at | 7.02 | 0.08 | 0.04715 |
| LEF1 | 210948_s_at | 7.02 | 0.07 | 0.15749 |
| HYAL3 | 211728_s_at | 7.02 | 0.04 | 0.01476 |
| PCOLCE2 | 219295_s_at | 7.02 | 0.06 | 0.00459 |
| HS3ST1 | 205466_s_at | 7.02 | 0.07 | 0.08931 |
| MFAP3L | 205442_at | 7.01 | 0.07 | 0.15456 |
| ESRRG | 209966_x_at | 7 | 0.05 | 0.00785 |
| KCNAB1 | 210079_x_at | 7 | 0.02 | 0.19704 |
| ABCB4 | 207819_s_at | 7 | 0.04 | 0.08178 |
| DNM3 | 209839_at | 7 | 0.08 | 0.00113 |
| SLC12A6 | 220740_s_at | 6.99 | 0.02 | 0.01249 |
| NRXN1 | 216096_s_at | 6.98 | 0.02 | 0.02706 |
| TNIK | 213109_at | 6.98 | 0.05 | 0.01074 |
| GLI2 | 207034_s_at | 6.93 | 0.03 | 0.00408 |
| AFF3 | 205735_s_at | 6.93 | 0.02 | 0.01012 |
| KCNAB1 | 210471_s_at | 6.92 | 0.02 | 0.16257 |
| DAB1 | 220611_at | 6.92 | 0.02 | 0.03573 |
| ANK2 | 216195_at | 6.92 | 0.04 | 0.09369 |
| TEC | 206301_at | 6.91 | 0.03 | 0.00424 |
| WISP1 | 206796_at | 6.9 | 0.07 | 0.02554 |
| NRXN1 | 209914_s_at | 6.9 | 0.02 | 0.07166 |
| MCTP1 | 220122_at | 6.9 | 0.08 | 0.00638 |
| FGF12 | 207501_s_at | 6.9 | 0.04 | 0.10060 |
| IL5RA | 207902_at | 6.89 | 0.02 | 0.00232 |
| AFF3 | 205734_s_at | 6.89 | 0.04 | 0.07308 |
| RAD52 | 211904_x_at | 6.89 | 0.02 | 0.09990 |
| HTR4 | 207577_at | 6.89 | 0.03 | 0.04897 |
| HS3ST1 | 213991_s_at | 6.88 | 0.02 | 0.00154 |
| FUT9 | 216185_at | 6.88 | 0.02 | 0.13109 |
| DYRK1A | 211079_s_at | 6.87 | 0.03 | 0.09784 |
| KCNAB1 | 210078_s_at | 6.86 | 0.05 | 0.05448 |
| NRP2 | 211844_s_at | 6.85 | 0.03 | 0.07661 |
| IL5RA | 211517_s_at | 6.84 | 0.04 | 0.11199 |
| PRKCE | 206248_at | 6.83 | 0.02 | 0.04497 |
| TBL1X | 213401_s_at | 6.82 | 0.02 | 0.04299 |
| SPATS2L | 215617_at | 6.79 | 0.06 | 0.00220 |
| ERBB4 | 206794_at | 6.79 | 0.05 | 0.04933 |
| TRIM48 | 220534_at | 6.78 | 0.03 | 0.04251 |
| ERC2 | 213938_at | 6.78 | 0.04 | 0.13941 |
| ARSE | 205894_at | 6.75 | 0.04 | 0.03859 |
| WISP1 | 211312_s_at | 6.75 | 0.02 | 0.05958 |
| RAD52 | 210630_s_at | 6.74 | 0.06 | 0.12087 |
| NRXN1 | 209915_s_at | 6.74 | 0.02 | 0.00186 |
| TLE4 | 216997_x_at | 6.72 | 0.03 | 0.00394 |
| CACNB2 | 213714_at | 6.7 | 0.03 | 0.09479 |
| SLC4A4 | 211494_s_at | 6.69 | 0.02 | 0.04014 |
| EPB41 | 214530_x_at | 6.67 | 0.02 | 0.11757 |
| PTGS2 | 204748_at | 6.66 | 0.1 | 0.07900 |
| LRCH1 | 214936_at | 6.65 | 0.02 | 0.19740 |
| LPHN3 | 209866_s_at | 6.62 | 0.09 | 0.02648 |
| SP4 | 206663_at | 6.6 | 0.02 | 0.03413 |
| MFAP3L | 210843_s_at | 6.58 | 0.03 | 0.05724 |
| NTNG1 | 206713_at | 6.56 | 0.02 | 0.15772 |
| GRM8 | 216992_s_at | 6.56 | 0.03 | 0.00917 |
| SNTG2 | 220487_at | 6.48 | 0.02 | 0.09169 |
| CCDC88A | 219387_at | 6.48 | 0.03 | 0.00943 |
| MFAP3L | 210492_at | 6.46 | 0.02 | 0.27020 |
| EPB41 | 207793_s_at | 6.43 | 0.02 | 0.14880 |
| CUL3 | 201372_s_at | 6.38 | 0.02 | 0.04445 |
| PTPRZ1 | 204469_at | 6.37 | 0.03 | 0.02128 |
| NRP2 | 210842_at | 6.37 | 0.02 | 0.01312 |
| PDZK1 | 205380_at | 6.32 | 0.09 | 0.00382 |
| DPPA4 | 219651_at | 6.32 | 0.07 | 0.06463 |
| SLC4A4 | 210738_s_at | 6.27 | 0.03 | 0.00763 |
| GRM8 | 216255_s_at | 6.26 | 0.03 | 0.11487 |
| GAP43 | 204471_at | 6.19 | 0.03 | 0.01214 |
| DBT | 211196_at | 6.18 | 0.02 | 0.02234 |
| CASC5 | 220247_at | 6.17 | 0.01 | 0.07876 |
| LRP1B | 219643_at | 6.14 | 0.03 | 0.00130 |
| IL5RA | 211516_at | 6.14 | 0.01 | 0.04613 |
| PCDH7 | 210941_at | 6.13 | 0.04 | 0.18964 |
| EFHC2 | 220523_at | 6.12 | 0.02 | 0.00047 |
| FUT9 | 214046_at | 6.08 | 0.07 | 0.13738 |
| FUT9 | 207696_at | 5.96 | 0.01 | 0.23998 |
| PDE6C | 211093_at | 5.91 | 0.01 | 0.00334 |

Median expr = median log expression value across the samples; CV = coefficient of variation of the log expression values; Surv. P value = survival p-value.

TABLE 4
Genes CN-invariant in the TCGA EOC samples and insignificant
for survival in both GSE9899 and TCGA patient cohorts.

| Symbol | Refseq | Chr | Start | End | Description |
|---|---|---|---|---|---|
| AFF3 | NM_001025108 | chr2 | 100163715 | 100722045 | AF4/FMR2 family |
| | | | | | member 3 isoform |
| | | | | | 2 |
| AMD1 | NM_001287216 | chr6 | 111195986 | 111216915 | S- |
| | | | | | adenosylmethionine |
| | | | | | decarboxylase |
| | | | | | proenzyme isoform |
| | | | | | 5 |
| ANK2 | NM_001127493 | chr4 | 113739238 | 114304896 | ankyrin-2 isoform 3 |
| ARHGAP10 | NM_024605 | chr4 | 148653452 | 148993927 | rho GTPase- |
| | | | | | activating protein |
| | | | | | 10 |
| ATF7IP2 | NM_024997 | chr16 | 10479911 | 10577495 | activating |
| | | | | | transcription factor |
| | | | | | 7-interacting |
| | | | | | protein 2 isoform 1 |
| BATF3 | NM_018664 | chr1 | 212859758 | 212873327 | basic leucine zipper |
| | | | | | transcriptional |
| | | | | | factor ATF-like 3 |
| BRE | NM_199194 | chr2 | 28113481 | 28561767 | BRCA1-A complex |
| | | | | | subunit BRE |
| | | | | | isoform 2 |
| CASC5 | NM_170589 | chr15 | 40886446 | 40954881 | protein CASC5 |
| | | | | | isoform 1 |
| CCDC88A | NM_018084 | chr2 | 55514977 | 55647057 | girdin isoform 2 |
| CLMN | NM_024734 | chr14 | 95648275 | 95786245 | calmin |
| CUL3 | NM_001257197 | chr2 | 225334866 | 225450114 | cullin-3 isoform 2 |
| DAPK1 | NM_001288729 | chr9 | 90113449 | 90323549 | death-associated |
| | | | | | protein kinase 1 |
| DEPDC1 | NM_001114120 | chr1 | 68939834 | 68962904 | DEP domain- |
| | | | | | containing protein |
| | | | | | 1A isoform a |
| EPHB2 | NM_004442 | chr1 | 23037330 | 23241823 | ephrin type-B |
| | | | | | receptor 2 isoform |
| | | | | | 2 precursor |
| ESRRG | NM_206594 | chr1 | 216676587 | 217262987 | estrogen-related |
| | | | | | receptor gamma |
| | | | | | isoform 2 |
| FGF12 | NM_004113 | chr3 | 191857181 | 192445388 | fibroblast growth |
| | | | | | factor 12 isoform 2 |
| FHL1 | NM_001159702 | chrX | 135229558 | 135293518 | four and a half LIM |
| | | | | | domains protein 1 |
| | | | | | isoform 1 |
| FUT9 | NM_006581 | chr6 | 96463844 | 96663488 | alpha-(1,3)- |
| | | | | | fucosyltransferase 9 |
| GBE1 | NM_000158 | chr3 | 81538849 | 81810950 | 1,4-alpha-glucan- |
| | | | | | branching enzyme |
| HTR4 | NM_199453 | chr5 | 147830594 | 148016624 | 5- |
| | | | | | hydroxytryptamine |
| | | | | | receptor 4 isoform |
| | | | | | g |
| HYAL3 | NM_003549 | chr3 | 50330258 | 50336899 | hyaluronidase-3 |
| | | | | | isoform 1 precursor |
| IL5RA | NM_175726 | chr3 | 3108007 | 3152058 | interleukin-5 |
| | | | | | receptor subunit |
| | | | | | alpha isoform 1 |
| | | | | | precursor |
| KCNAB1 | NM_172159 | chr3 | 156008775 | 156256927 | voltage-gated |
| | | | | | potassium channel |
| | | | | | subunit beta-1 |
| | | | | | isoform 3 |
| LDB2 | NM_001290 | chr4 | 16503164 | 16900424 | LIM domain- |
| | | | | | binding protein 2 |
| | | | | | isoform a |
| LEF1 | NM_001130714 | chr4 | 108968700 | 109090112 | lymphoid enhancer- |
| | | | | | binding factor 1 |
| | | | | | isoform 3 |
| LRCH1 | NM_015116 | chr13 | 47127295 | 47319036 | leucine-rich repeat |
| | | | | | and calponin |
| | | | | | homology domain- |
| | | | | | containing protein 1 |
| | | | | | isoform 2 |
| MFAP3L | NM_021647 | chr4 | 170907747 | 170947581 | microfibrillar- |
| | | | | | associated protein |
| | | | | | 3-like isoform 1 |
| | | | | | precursor |
| MTR | NM_001291939 | chr1 | 236958580 | 237067281 | methionine |
| | | | | | synthase isoform 2 |
| NMD3 | NM_015938 | chr3 | 160939098 | 160969795 | 60S ribosomal |
| | | | | | export protein |
| | | | | | NMD3 |
| NOTCH2 | NM_024408 | chr1 | 120454175 | 120612317 | neurogenic locus |
| | | | | | notch homolog |
| | | | | | protein 2 isoform 1 |
| | | | | | preproprotein |
| NRP2 | NM_018534 | chr2 | 206547223 | 206641880 | neuropilin-2 |
| | | | | | isoform 4 precursor |
| NTNG1 | NM_014917 | chr1 | 107682744 | 108024475 | netrin-G1 isoform 3 |
| | | | | | precursor |
| PARN | NM_001134477 | chr16 | 14529556 | 14724128 | poly(A)-specific |
| | | | | | ribonuclease PARN |
| | | | | | isoform 2 |
| PRKCZ | NM_001033582 | chr1 | 2036154 | 2116834 | protein kinase C |
| | | | | | zeta type isoform 2 |
| PRUNE | NM_021222 | chr1 | 150980972 | 151008189 | protein prune |
| | | | | | homolog isoform 1 |
| PUM1 | NM_014676 | chr1 | 31404352 | 31538564 | pumilio homolog 1 |
| | | | | | isoform 2 |
| RNF144A | NM_014746 | chr2 | 7057522 | 7184309 | E3 ubiquitin- |
| | | | | | protein ligase |
| | | | | | RNF144A |
| SCHIP1 | NM_014575 | chr3 | 158991035 | 159615155 | schwannomin- |
| | | | | | interacting protein |
| | | | | | 1 isoform 1 |
| SLC12A6 | NM_001042495 | chr15 | 34522196 | 34630265 | solute carrier |
| | | | | | family 12 member |
| | | | | | 6 isoform c |
| SLC4A4 | NM_003759 | chr4 | 72204769 | 72437804 | electrogenic |
| | | | | | sodium bicarbonate |
| | | | | | cotransporter 1 |
| | | | | | isoform 2 |
| SP4 | NM_003112 | chr7 | 21467688 | 21554151 | transcription factor |
| | | | | | Sp4 |
| TBL1X | NM_001139468 | chrX | 9431334 | 9687780 | F-box-like/WD |
| | | | | | repeat-containing |
| | | | | | protein TBL1X |
| | | | | | isoform b |
| TLE4 | NM_007005 | chr9 | 82186687 | 82341796 | transducin-like |
| | | | | | enhancer protein 4 |
| | | | | | isoform 3 |
| TNIK | NM_001161561 | chr3 | 170780291 | 171178197 | TRAF2 and NCK- |
| | | | | | interacting protein |
| | | | | | kinase isoform 3 |
| TSPAN9 | NM_001168320 | chr12 | 3186520 | 3395730 | tetraspanin-9 |
| WDFY3 | NM_014991 | chr4 | 85590692 | 85887544 | WD repeat and |
| | | | | | FYVE domain- |
| | | | | | containing protein 3 |
| ZNF274 | NM_133502 | chr19 | 58694355 | 58724928 | neurotrophin |
| | | | | | receptor-interacting |
| | | | | | factor homolog |
| | | | | | isoform c |
| ZNF702P | NR_003578 | chr19 | 53471503 | 53496784 | |

TABLE 5
Genes that are CN-invariant in normal human tissues,
located in CN-invariant cytobands of EOC tumors.

| Symbol | Refseq | Chr | Start | End | Description |
|---|---|---|---|---|---|
| AZIN2 | NM_052998 | chr1 | 33546713 | 33586132 | antizyme inhibitor |
| | | | | | 2 isoform 1 |
| BATF3 | NM_018664 | chr1 | 212859758 | 212873327 | basic leucine zipper |
| | | | | | transcriptional |
| | | | | | factor ATF-like 3 |
| DEPDC1 | NM_001114120 | chr1 | 68939834 | 68962904 | DEP domain- |
| | | | | | containing protein |
| | | | | | 1A isoform a |
| EHD3 | NM_014600 | chr2 | 31456879 | 31491260 | EH domain- |
| | | | | | containing protein |
| | | | | | 3 |
| FAHD2A | NM_016044 | chr2 | 96068447 | 96078879 | fumarylacetoacetate |
| | | | | | hydrolase domain- |
| | | | | | containing protein |
| | | | | | 2A |
| FAM132B | NM_001291832 | chr2 | 239067648 | 239077532 | erythroferrone |
| | | | | | precursor |
| FHL2 | NM_201557 | chr2 | 105977282 | 106055230 | four and a half LIM |
| | | | | | domains protein 2 |
| HS3ST1 | NM_005114 | chr4 | 11399987 | 11430537 | heparan sulfate |
| | | | | | glucosamine 3-O- |
| | | | | | sulfotransferase 1 |
| | | | | | precursor |
| IDO2 | NM_194294 | chr8 | 39792473 | 39873910 | indoleamine 2,3- |
| | | | | | dioxygenase 2 |
| LIN54 | NM_001115008 | chr4 | 83845756 | 83931987 | protein lin-54 |
| | | | | | homolog isoform b |
| LINC00578 | NR_047568 | chr3 | 177159708 | 177470492 | |
| LINC00882 | NR_028303 | chr3 | 106828636 | 106959485 | |
| LINC01001 | NR_028326 | chr11 | 126986 | 131920 | |
| LINC01091 | NR_027106 | chr4 | 124695418 | 124786730 | |
| LMCD1-AS1 | NR_033378 | chr3 | 8262833 | 8543344 | |
| LOC100506457 | NR_110198 | chr2 | 12147241 | 12223743 | |
| LOC101926942 | NR_110657 | chr10 | 92162277 | 92300562 | |
| LOC101927905 | NR_120455 | chr12 | 8388010 | 8391553 | |
| LOC391003 | NM_001099850 | chr1 | 13035498 | 13039011 | PRAME family |
| | | | | | member-like |
| LOC440700 | NR_036683 | chr1 | 165667986 | 165679199 | |
| LOC729970 | NR_033998 | chr1 | 95393583 | 95428826 | |
| MALRD1 | NM_001142308 | chr10 | 19337699 | 20023407 | MAM and LDL- |
| | | | | | receptor class A |
| | | | | | domain-containing |
| | | | | | protein 1 precursor |
| MIR5694 | NR_049879 | chr10 | 122344590 | 122806858 | |
| MRPL47 | NM_020409 | chr3 | 179306254 | 179322434 | 39S ribosomal |
| | | | | | protein L47, |
| | | | | | mitochondrial |
| | | | | | isoform a |
| NAA16 | NM_024561 | chr13 | 41885340 | 41951166 | N-alpha- |
| | | | | | acetyltransferase |
| | | | | | 16, NatA auxiliary |
| | | | | | subunit isoform 1 |
| NBPF8 | NR_102404 | chr1 | 147574322 | 148346929 | |
| NMD3 | NM_015938 | chr3 | 160939098 | 160969795 | 60S ribosomal |
| | | | | | export protein |
| | | | | | NMD3 |
| NUP133 | NM_018230 | chr1 | 229577043 | 229644088 | nuclear pore |
| | | | | | complex protein |
| | | | | | Nup133 |
| NYAP2 | NM_020864 | chr2 | 226265601 | 226518734 | neuronal tyrosine- |
| | | | | | phosphorylated |
| | | | | | phosphoinositide-3- |
| | | | | | kinase adapter 2 |
| PTCHD1-AS | NR_073010 | chrX | 22277913 | 23311263 | |
| RAI2 | NM_001172743 | chrX | 17818168 | 17879457 | retinoic acid- |
| | | | | | induced protein 2 |
| | | | | | isoform 1 |
| RGS18 | NM_130782 | chr1 | 192127591 | 192154945 | regulator of G- |
| | | | | | protein signaling 18 |
| SEPSECS-AS1 | NR_037934 | chr4 | 25162293 | 25200127 | |
| SRGAP2C | NM_001271872 | chr1 | 206516199 | 206581301 | SLIT-ROBO Rho |
| | | | | | GTPase-activating |
| | | | | | protein 2C |
| TC2N | NM_152332 | chr14 | 92246095 | 92302870 | tandem C2 domains |
| | | | | | nuclear protein |
| | | | | | isoform 1 |
| TCEANC2 | NM_153035 | chr1 | 54519273 | 54565416 | transcription |
| | | | | | elongation factor A |
| | | | | | N-terminal and |
| | | | | | central domain- |
| | | | | | containing protein |
| | | | | | 2 |
| TENM3 | NM_001080477 | chr4 | 183245136 | 183724177 | teneurin-3 |
| TEX41 | NR_033870 | chr2 | 145425533 | 145834291 | |
| TGFBRAP1 | NM_004257 | chr2 | 105880846 | 105946171 | transforming |
| | | | | | growth factor-beta |
| | | | | | receptor-associated |
| | | | | | protein 1 |
| WISP1 | NM_080838 | chr8 | 134203281 | 134243932 | WNT1-inducible- |
| | | | | | signaling pathway |
| | | | | | protein 1 isoform 2 |
| | | | | | precursor |
| YEATS2 | NM_018023 | chr3 | 183415605 | 183530413 | YEATS domain- |
| | | | | | containing protein |
| | | | | | 2 |

TABLE 6
Primers.

| | | | | |
|---|---|---|---|---|
| Target gene | Forward | SEQ ID NO | Reverse | SEQ ID NO |

Primer set 1

| | | | | |
|---|---|---|---|---|
| XRCC5 | AGGTCGTGGATGTATGGGGA | 1 | GGCCGCATCCAACTTGTTTT | 2 |
| AUTS2 | GTAAGGTGCACGTTTCCTGA | 3 | CTCTAACTCGCGATGGCTCC | 4 |
| EIF5 | ACCGAGAACTCTTGCAGTCG | 5 | AGAACTGGTCTGACACGCTG | 6 |
| PARN | CCCACCATAGCTGCCTGAAA | 7 | CATACGGCAAGCCCTCTCAT | 8 |
| YEATS2 | CCCGAGTGCCCATCATCATT | 9 | CCTTCTGTACTTGCAGCCCT | 10 |
| FHL2 | GAAGTGCTCCCTCTCACTGG | 11 | GCAAGATTGCCTGGGTGAGA | 12 |

Primer set 2

| | | | | |
|---|---|---|---|---|
| XRCC5 | ACCAAGTGGAGACACAGCAG | 13 | TCCCCATACATCCACGACCT | 14 |
| AUTS2 | TGTAAGGTGCACGTTTCCTG | 15 | AGGTTGACCTGTTACGGCTG | 16 |
| EIF5 | CTGTCAATGTCAACCGCAGC | 17 | GCCTTTGCAACGTCAACCAT | 18 |
| PARN | GTGGCGCTGTGTTCACTTTC | 19 | AATGGGCTGGGACATGTTGT | 20 |
| YEATS2 | AGGAATGACGGGGACTCCAT | 21 | AATGATGATGGGCACTCGGG | 22 |
| FHL2 | TCGAGTAAGGCACACCCAAA | 23 | TAGACTTGACGCAACGGGAG | 24 |

TABLE 7
Worldwide ten most frequent cancers used in the present
examples. The samples data has been obtained from TCGA

| Name | Frequency, % | Sample size |
|---|---|---|
| Breast invasive carcinoma | 12 | 1096 |
| Ovarian serous adenocarcinoma | 1.7 | 593 |
| Head and neck squamous cell | 5 | 524 |
| carcinoma | | |
| Lung adenocarcinoma | 2.5 | 518 |
| Lung squamous cell carcinoma | 6.6 | 501 |
| Prostate adenocarcinoma | 7.9 | 493 |
| Colon adenocarcinoma | 9.5 | 454 |
| Stomach adenocarcinoma | 6.1 | 442 |
| Liver hepatocellular carcinoma | 4.5 | 372 |
| Cervical squamous cell carcinoma | 3.1 | 297 |

TABLE 8
The candidate reference loci for use with the 10 most frequent cancers listed in Table 7.

| Symbol | Refseq | Chr | Start | End | Description |
|---|---|---|---|---|---|
| ALG10 | NM_032834 | chr12 | 34175215 | 34181236 | dol-P- |
| | | | | | Glc:Glc(2)Man(9)GlcNAc(2)- |
| | | | | | PP-Dol alpha-1,2- |
| | | | | | glucosyltransferase |
| ANKRD20A9P | NR_027995 | chr13 | 19408542 | 19446109 | |
| AUTS2 | NM_015570 | chr7 | 69063904 | 70258054 | autism susceptibility gene 2 |
| | | | | | protein isoform 1 |
| BAGE | NM_001187 | chr21 | 11057795 | 11098937 | B melanoma antigen 1 precursor |
| BAGE2 | NM_182482 | chr21 | 11020841 | 11098925 | B melanoma antigen 2 precursor |
| BAGE3 | NM_182481 | chr21 | 11020841 | 11098925 | B melanoma antigen 3 precursor |
| BAGE4 | NM_181704 | chr21 | 11020841 | 11098925 | B melanoma antigen 4 precursor |
| BAGE5 | NM_182484 | chr21 | 11020841 | 11098925 | B melanoma antigen 5 precursor |
| CALN1 | NM_001017440 | chr7 | 71244475 | 71802208 | calcium-binding protein 8 |
| | | | | | isoform 2 |
| CDH12 | NM_004061 | chr5 | 21750972 | 22853731 | cadherin-12 preproprotein |
| CDH18 | NM_004934 | chr5 | 19473154 | 19988353 | cadherin-18 isoform 1 |
| | | | | | preproprotein |
| CHEK2P2 | NR_038836 | chr15 | 20487996 | 20496811 | |
| CNTNAP3B | NM_001201380 | chr9 | 43684884 | 43922473 | contactin-associated protein-like |
| | | | | | 3B precursor |
| CNTNAP3P2 | NR_111893 | chr9 | 43685195 | 43921493 | |
| CSMD1 | NM_033225 | chr8 | 2792874 | 4852328 | CUB and sushi domain- |
| | | | | | containing protein 1 precursor |
| DDX3Y | NM_001122665 | chrY | 15016018 | 15030439 | ATP-dependent RNA helicase |
| | | | | | DDX3Y isoform 1 |
| FAM133A | NM_173698 | chrX | 92929011 | 92967273 | protein FAM133A |
| FAM135B | NM_015912 | chr8 | 139142265 | 139509065 | protein FAM135B |
| FAM27C | NR_027421 | chr9 | 44990235 | 44991492 | |
| FAM27E2 | NR_103714 | chr9 | 46385603 | 46387373 | |
| FAM74A1 | NR_026803 | chr9 | 65488295 | 65494240 | |
| FAM74A4 | NR_110998 | chr9 | 65487272 | 65494386 | |
| FAM74A6 | NR_110999 | chr9 | 65488295 | 65494240 | |
| GBE1 | NM_000158 | chr3 | 81538849 | 81810950 | 1,4-alpha-glucan-branching |
| | | | | | enzyme |
| GUSBP1 | NR_027028 | chr5 | 21459588 | 21497305 | |
| GYG2P1 | NR_033667 | chrY | 14517914 | 14533389 | |
| HERC2P3 | NR_036432 | chr15 | 20613649 | 20711433 | |
| KGFLP1 | NR_003674 | chr9 | 46687556 | 46746820 | |
| KHDRBS3 | NM_006558 | chr8 | 136469715 | 136659848 | KH domain-containing, RNA- |
| | | | | | binding, signal transduction- |
| | | | | | associated protein 3 |
| LINC00417 | NR_047508 | chr13 | 19312239 | 19314239 | |
| LINC01189 | NR_046203 | chr9 | 46763790 | 46833319 | |
| LOC100507468 | NR_108105 | chr7 | 69061123 | 69062481 | |
| LOC101927827 | NR_121564 | chr9 | 44384584 | 44391314 | |
| LOC101928201 | NR_110390 | chrX | 4545240 | 4551613 | |
| LOC102723427 | NR_120514 | chr7 | 67485239 | 67497677 | |
| MIR3648-1 | NR_037421 | chr21 | 9825831 | 9826011 | |
| MIR3687-1 | NR_037458 | chr21 | 9826202 | 9826263 | |
| MIR3914-1 | NR_037477 | chr7 | 70772657 | 70772756 | |
| MIR3914-2 | NR_037479 | chr7 | 70772659 | 70772754 | |
| MIR4275 | NR_036237 | chr4 | 28821203 | 28821290 | |
| MIR4650-1 | NR_039793 | chr7 | 72162873 | 72162949 | |
| MIR4650-2 | NR_039794 | chr7 | 72162873 | 72162949 | |
| NAP1L3 | NM_004538 | chrX | 92925924 | 92928682 | nucleosome assembly protein 1- |
| | | | | | like 3 |
| NLGN4X | NM_181332 | chrX | 5808066 | 6146923 | neuroligin-4, X-linked |
| PCDH11X | NM_032968 | chrX | 91090459 | 91878228 | protocadherin-11 X-linked |
| | | | | | isoform c precursor |
| PCDH7 | NM_032456 | chr4 | 30722029 | 30726957 | protocadherin-7 isoform b |
| | | | | | precursor |
| PCDH9 | NM_203487 | chr13 | 66876965 | 67804468 | protocadherin-9 isoform 1 |
| | | | | | precursor |
| PCDH9-AS2 | NR_046527 | chr13 | 67399300 | 67489163 | |
| PCDH9-AS3 | NR_046636 | chr13 | 67551520 | 67559908 | |
| PCDH9-AS4 | NR_046637 | chr13 | 67565017 | 67576132 | |
| PFKP | NM_001242339 | chr10 | 3110818 | 3178997 | ATP-dependent 6- |
| | | | | | phosphofructokinase, platelet |
| | | | | | type isoform 2 |
| PITRM1 | NM_014889 | chr10 | 3179918 | 3215033 | presequence protease, |
| | | | | | mitochondrial isoform 2 |
| | | | | | precursor |
| PITRM1-AS1 | NR_038284 | chr10 | 3183792 | 3190821 | |
| PMCHL1 | NR_003921 | chr5 | 22142460 | 22152379 | |
| PXDNL | NM_144651 | chr8 | 52232136 | 52722005 | peroxidasin-like protein |
| | | | | | precursor |
| ROBO1 | NM_133631 | chr3 | 78646387 | 79068609 | roundabout homolog 1 isoform |
| | | | | | b |
| SPATA31A5 | NM_001113541 | chr9 | 65503362 | 65509610 | spermatogenesis-associated |
| | | | | | protein 31A5 |
| SPATA31A6 | NM_001145196 | chr9 | 43624501 | 43630730 | spermatogenesis-associated |
| | | | | | protein 31A6 |
| SPATA31A7 | NM_015667 | chr9 | 65503365 | 65509610 | spermatogenesis-associated |
| | | | | | protein 31A7 |
| SYT10 | NM_198992 | chr12 | 33528347 | 33592754 | synaptotagmin-10 |
| TEKT4P2 | NR_038329 | chr21 | 9915249 | 9968594 | |
| TPTE | NM_199259 | chr21 | 10906186 | 10990943 | putative tyrosine-protein |
| | | | | | phosphatase TPTE isoform beta |
| TTTY15 | NR_001545 | chrY | 14774297 | 14804153 | |
| TYW1B | NM_001145440 | chr7 | 72039491 | 72298813 | S-adenosyl-L-methionine- |
| | | | | | dependent tRNA 4- |
| | | | | | demethylwyosine synthase |
| USP9Y | NM_004654 | chrY | 14813159 | 14972768 | probable ubiquitin carboxyl- |
| | | | | | terminal hydrolase FAF-Y |
| WBSCR17 | NM_022479 | chr7 | 70597522 | 71178586 | putative polypeptide N- |
| | | | | | acetylgalactosaminyltransferase- |
| | | | | | like protein 3 |

TABLE 9
The candidate reference loci for use with cancer-unaffected
tissue samples collected from cancer patients.

| Symbol | Refseq | Chr | Start | End | Description |
|---|---|---|---|---|---|
| AKAP17A | NR_027383 | chrY | 1660485 | 1671407 | |
| ASMT | NM_001171038 | chrY | 1683940 | 1711974 | acetylserotonin O- |
| | | | | | methyltransferase |
| | | | | | isoform 1 |
| ASMTL | NM_004192 | chrY | 1472031 | 1521870 | N-acetylserotonin |
| | | | | | O- |
| | | | | | methyltransferase- |
| | | | | | like protein |
| | | | | | isoform 1 |
| ASMTL-AS1 | NR_026711 | chrY | 1469423 | 1484314 | |
| CD99P1 | NR_033380 | chrY | 2477305 | 2525270 | |
| CRLF2 | NM_001012288 | chrY | 1264893 | 1281616 | cytokine receptor- |
| | | | | | like factor 2 |
| | | | | | isoform 2 |
| DDX11L16 | NR_110561 | chrY | 59358328 | 59360854 | |
| IL3RA | NM_002183 | chrY | 1405508 | 1451582 | interleukin-3 |
| | | | | | receptor subunit |
| | | | | | alpha isoform 1 |
| | | | | | precursor |
| IL9R | NM_002186 | chrY | 59330251 | 59343488 | interleukin-9 |
| | | | | | receptor isoform 1 |
| | | | | | precursor |
| LINC00685 | NR_027231 | chrY | 231384 | 232054 | |
| MIR3690 | NR_037461 | chrY | 1362810 | 1362885 | |
| MIR6089 | NR_106737 | chrY | 2477231 | 2477295 | |
| P2RY8 | NM_178129 | chrY | 1531465 | 1606037 | P2Y purinoceptor |
| | | | | | 8 |
| SLC25A6 | NM_001636 | chrY | 1455044 | 1461039 | ADP/ATP |
| | | | | | translocase 3 |
| SLTM | NM_001013843 | chr15 | 59171243 | 59225852 | SAFB-like |
| | | | | | transcription |
| | | | | | modulator isoform |
| | | | | | b |
| ZBED1 | NM_004729 | chrY | 2354454 | 2369008 | zinc finger BED |
| | | | | | domain- |
| | | | | | containing protein |
| | | | | | 1 |

TABLE 10
The candidate reference loci for use with tissue samples collected from healthy
subjects and patients with myocardial infarction (non-tumor disease).

| Symbol | Refseq | Chr | Start | End | Description |
|---|---|---|---|---|---|
| ABCB7 | NM_004299 | chrX | 74273006 | 74376175 | ATP-binding cassette sub- |
| | | | | | family B member 7, |
| | | | | | mitochondrial isoform 1 |
| ABCD1 | NM_000033 | chrX | 152990322 | 153010216 | ATP-binding cassette sub- |
| | | | | | family D member 1 |
| ACE2 | NM_021804 | chrX | 15579155 | 15620192 | angiotens in-converting |
| | | | | | enzyme 2 precursor |
| ACTRT1 | NM_138289 | chrX | 127184940 | 127186382 | actin-related protein T1 |
| AKAP4 | NM_139289 | chrX | 49955419 | 49965004 | A-kinase anchor protein 4 |
| | | | | | isoform 2 |
| ALAS2 | NM_001037968 | chrX | 55035487 | 55057497 | 5-aminolevulinate synthase, |
| | | | | | erythroid-specific, |
| | | | | | mitochondrial isoform c |
| | | | | | precursor |
| ALG13 | NM_001099922 | chrX | 110924345 | 111003875 | putative bifunctional UDP- |
| | | | | | N-acetylglucosamine |
| | | | | | transferase and |
| | | | | | deubiquitinase ALG13 |
| | | | | | isoform 1 |
| AMELX | NM_001142 | chrX | 11311532 | 11318881 | amelogenin, X isoform |
| | | | | | isoform 1 precursor |
| AMELY | NM_001143 | chrY | 6733958 | 6742068 | amelogenin, Y isoform |
| | | | | | precursor |
| AMER1 | NM_152424 | chrX | 63404996 | 63425624 | APC membrane recruitment |
| | | | | | protein 1 |
| AMOT | NM_001113490 | chrX | 112018104 | 112066354 | angiomotin isoform 1 |
| ANHX | NM_001191054 | chr12 | 133794897 | 133812422 | anomalous homeobox |
| | | | | | protein |
| AP1S2 | NM_003916 | chrX | 15843928 | 15873137 | AP-1 complex subunit |
| | | | | | sigma-2 isoform 2 |
| APEX2 | NM_014481 | chrX | 55026755 | 55034306 | DNA-(apurinic or |
| | | | | | apyrimidinic site) lyase 2 |
| | | | | | isoform 1 |
| APOO | NR_026545 | chrX | 23851464 | 23926057 | |
| APOOL | NM_198450 | chrX | 84258897 | 84348323 | MICOS complex subunit |
| | | | | | MIC27 precursor |
| ARAF | NM_001256197 | chrX | 47420498 | 47425373 | serine/threonine-protein |
| | | | | | kinase A-Raf isoform 3 |
| ARHGAP4 | NM_001666 | chrX | 153172829 | 153191714 | rho GTPase-activating |
| | | | | | protein 4 isoform 2 |
| ARHGEF6 | NM_004840 | chrX | 135747711 | 135863503 | rho guanine nucleotide |
| | | | | | exchange factor 6 |
| ARHGEF9 | NM_001173480 | chrX | 62854847 | 62975031 | rho guanine nucleotide |
| | | | | | exchange factor 9 isoform 3 |
| ARHGEF9-IT1 | NR_046803 | chrX | 62890075 | 62891382 | |
| ARMCX1 | NM_016608 | chrX | 100805513 | 100809675 | armadillo repeat-containing |
| | | | | | X-linked protein 1 |
| ARMCX4 | NR_028407 | chrX | 100673250 | 100790975 | |
| ARX | NM_139058 | chrX | 25021812 | 25034065 | homeobox protein ARX |
| ATG4A | NM_178270 | chrX | 107334898 | 107397901 | cysteine protease ATG4A |
| | | | | | isoform b |
| ATP2B3 | NM_021949 | chrX | 152801579 | 152848387 | plasma membrane calcium- |
| | | | | | transporting ATPase 3 |
| | | | | | isoform 3a |
| ATP7A | NM_001282224 | chrX | 77166152 | 77305892 | copper-transporting ATPase |
| | | | | | 1 isoform 2 |
| ATRX | NM_000489 | chrX | 76760355 | 77041755 | transcriptional regulator |
| | | | | | ATRX isoform 1 |
| ATXN3L | NM_001135995 | chrX | 13336767 | 13338518 | putative ataxin-3-like protein |
| AVPR2 | NR_027419 | chrX | 153167984 | 153172620 | |
| AWAT2 | NM_001002254 | chrX | 69260391 | 69269788 | acyl-CoA wax alcohol |
| | | | | | acyltransferase 2 |
| BEX1 | NM_018476 | chrX | 102317580 | 102319168 | protein BEX 1 |
| BEX2 | NM_032621 | chrX | 102564273 | 102565974 | protein BEX2 isoform 3 |
| BEX4 | NM_001127688 | chrX | 102470019 | 102472128 | protein BEX4 |
| BEX5 | NM_001159560 | chrX | 101408678 | 101410762 | protein BEX5 |
| BMP 15 | NM_005448 | chrX | 50653734 | 50659641 | bone morphogenetic protein |
| | | | | | 15 precursor |
| BRDTP1 | NR_003539 | chrX | 95592084 | 95592901 | |
| BRS3 | NM_001727 | chrX | 135570124 | 135574598 | bombesin receptor subtype-3 |
| C1GALT1C1 | NM_001011551 | chrX | 119759528 | 119764005 | C1GALT1-specific |
| | | | | | chaperone 1 |
| CA5B | NM_007220 | chrX | 15756411 | 15805748 | carbonic anhydrase 5B, |
| | | | | | mitochondrial precursor |
| CA5BP1 | NR_026551 | chrX | 15693038 | 15721474 | |
| CAPN6 | NM_014289 | chrX | 110488326 | 110513774 | calpain-6 |
| CCDC160 | NM_001101357 | chrX | 133371076 | 133379808 | coiled-coil domain- |
| | | | | | containing protein 160 |
| CCNB3 | NM_033670 | chrX | 50027539 | 50094911 | G2/mitotic-specific cyclin- |
| | | | | | B3 isoform 1 |
| CD40LG | NM_000074 | chrX | 135730335 | 135742549 | CD40 ligand |
| CDK16 | NM_001170460 | chrX | 47082416 | 47089394 | cyclin-dependent kinase 16 |
| | | | | | isoform 3 |
| CDR1 | NM_004065 | chrX | 139865424 | 139866723 | cerebellar degeneration- |
| | | | | | related antigen 1 |
| CDX4 | NM_005193 | chrX | 72667089 | 72674421 | homeobox protein CDX-4 |
| CDY1 | NM_170723 | chrY | 27768263 | 27770485 | testis-specific |
| | | | | | chromodomain protein Y1 |
| | | | | | isoform a |
| CDY1B | NM_001003894 | chrY | 27768263 | 27770485 | testis-specific |
| | | | | | chromodomain protein Y1 |
| | | | | | isoform a |
| CDY2A | NM_004825 | chrY | 20137666 | 20139626 | testis-specific |
| | | | | | chromodomain protein Y2 |
| CDY2B | NM_001001722 | chrY | 20137667 | 20139627 | testis-specific |
| | | | | | chromodomain protein Y2 |
| CENPI | NM_006733 | chrX | 100354797 | 100417978 | centromere protein I |
| CENPVP1 | NR_033772 | chrX | 51453924 | 51455226 | |
| CENPVP2 | NR_033773 | chrX | 51453924 | 51455226 | |
| CHDC2 | NM_173695 | chrX | 36065052 | 36163187 | calponin homology domain- |
| | | | | | containing protein 2 |
| CHMP1B2P | NR_110646 | chrX | 79483987 | 79590817 | |
| CMC4 | NM_001018024 | chrX | 154289899 | 154299547 | cx9C motif-containing |
| | | | | | protein 4 |
| CSAG1 | NM_001102576 | chrX | 151903226 | 151909518 | putative chondrosarcoma- |
| | | | | | associated gene 1 protein |
| CSAG3 | NM_001129828 | chrX | 151927733 | 151928738 | chondrosarcoma-associated |
| | | | | | gene 2/3 protein isoform b |
| CSAG4 | NR_073432 | chrX | 151895977 | 151903136 | |
| CSPG4P1Y | NR_001554 | chrY | 27629054 | 27632852 | |
| CT45A10 | NM_001291527 | chrX | 134945650 | 134953901 | cancer/testis antigen family |
| | | | | | 45 member A-like |
| CT45A7 | NM_001291543 | chrX | 134963218 | 134971043 | cancer/testis antigen family |
| | | | | | 45 member A5-like |
| CT45A8 | NM_001291535 | chrX | 134866213 | 134874249 | cancer/testis antigen family |
| | | | | | 45 member A2-like |
| CT45A9 | NM_001291540 | chrX | 134866213 | 134874249 | cancer/testis antigen family |
| | | | | | 45 member A2-like |
| CT47A12 | NM_001242922 | chrX | 120072555 | 120075873 | cancer/testis antigen 47A |
| CT55 | NM_017863 | chrX | 134290460 | 134305751 | cancer/testis antigen 55 |
| | | | | | isoform 2 precursor |
| CT83 | NM_001017978 | chrX | 115592852 | 115594194 | kita-kyushu lung cancer |
| | | | | | antigen 1 |
| CUL4B | NM_001079872 | chrX | 119658445 | 119694817 | cullin-4B isoform 2 |
| CXorf23 | NM_198279 | chrX | 19930979 | 19988382 | uncharacterized protein |
| | | | | | CXorf23 |
| CXorf51B | NM_001244892 | chrX | 145895621 | 145896249 | uncharacterized protein |
| | | | | | LOC100133053 |
| CXorf58 | NM_152761 | chrX | 23926122 | 23957624 | putative uncharacterized |
| | | | | | protein CXorf58 isoform 1 |
| CXorf66 | NM_001013403 | chrX | 139037883 | 139047677 | uncharacterized protein |
| | | | | | CXorf66 precursor |
| CXorf67 | NM_203407 | chrX | 51149766 | 51151689 | uncharacterized protein |
| | | | | | CXorf67 |
| CYBB | NM_000397 | chrX | 37639269 | 37672714 | cytochrome b-245 heavy |
| | | | | | chain |
| CYLC1 | NM_001271680 | chrX | 83116133 | 83141708 | cylicin-1 isoform 2 |
| CYSLTR1 | NM_001282187 | chrX | 77526968 | 77583188 | cysteinyl leukotriene |
| | | | | | receptor 1 |
| DCX | NM_178152 | chrX | 110537006 | 110655460 | neuronal migration protein |
| | | | | | doublecortin isoform b |
| DDX11L1 | NR_046018 | chr1 | 11873 | 14409 | |
| DDX11L16 | NR_110561 | chrY | 59358328 | 59360854 | |
| DDX11L5 | NR_051986 | chr9 | 11986 | 14525 | |
| DDX26B-AS1 | NR_046740 | chrX | 134654007 | 134654599 | |
| DDX3Y | NM_001122665 | chrY | 15016018 | 15030439 | ATP-dependent RNA |
| | | | | | helicase DDX3Y isoform 1 |
| DDX53 | NM_182699 | chrX | 23018077 | 23020206 | DEAD box protein 53 |
| DIAPH2-AS1 | NR_125391 | chrX | 96783362 | 96819534 | |
| DKC1 | NR_110021 | chrX | 153991016 | 154005964 | |
| DLG3-AS1 | NR_109801 | chrX | 69672805 | 69675844 | |
| DMRTC1 | NM_033053 | chrX | 72091858 | 72095622 | doublesex- and mab-3- |
| | | | | | related transcription factor |
| | | | | | C1 |
| DMRTC1B | NM_001080851 | chrX | 72091858 | 72095622 | doublesex- and mab-3- |
| | | | | | related transcription factor |
| | | | | | C1 |
| DUSP21 | NM_022076 | chrX | 44703248 | 44704134 | dual specificity protein |
| | | | | | phosphatase 21 |
| DUSP9 | NM_001395 | chrX | 152907896 | 152916781 | dual specificity protein |
| | | | | | phosphatase 9 |
| EDA2R | NM_001242310 | chrX | 65815481 | 65835872 | tumor necrosis factor |
| | | | | | receptor superfamily |
| | | | | | member 27 isoform 2 |
| EGFL6 | NM_015507 | chrX | 13587693 | 13651694 | epidermal growth factor-like |
| | | | | | protein 6 isoform 1 |
| | | | | | precursor |
| EIF1AX | NM_001412 | chrX | 20142635 | 20159966 | eukaryotic translation |
| | | | | | initiation factor 1A, X- |
| | | | | | chromosomal |
| EIF1AX-AS1 | NR_046592 | chrX | 20158085 | 20158562 | |
| ELK1 | NM_001114123 | chrX | 47494918 | 47510003 | ETS domain-containing |
| | | | | | protein Elk-1 isoform a |
| ERCC6L | NM_017669 | chrX | 71424506 | 71458858 | DNA excision repair protein |
| | | | | | ERCC-6-like |
| ESX1 | NM_153448 | chrX | 103494718 | 103499599 | homeobox protein ESX1 |
| FAM120C | NM_017848 | chrX | 54094835 | 54209691 | constitutive coactivator of |
| | | | | | PPAR-gamma-like protein 2 |
| | | | | | isoform 1 |
| FAM122B | NM_001166599 | chrX | 133903595 | 133931185 | protein FAM122B isoform 2 |
| FAM122C | NM_001170781 | chrX | 133941222 | 133945211 | protein FAM122C isoform 4 |
| FAM133A | NM_173698 | chrX | 92929011 | 92967273 | protein FAM133A |
| FAM156A | NM_001242489 | chrX | 52976463 | 53024651 | protein |
| | | | | | FAM156A/FAM156B |
| FAM156B | NM_001099684 | chrX | 52976463 | 52985629 | protein |
| | | | | | FAM156A/FAM156B |
| FAM197Y2 | NR_001553 | chrY | 9316661 | 9322263 | |
| FAM197Y5 | NR_046300 | chrY | 9316661 | 9322263 | |
| FAM199X | NM_207318 | chrX | 103411155 | 103440582 | protein FAM199X |
| FAM223A | NR_027401 | chrX | 153799478 | 153800188 | |
| FAM223B | NR_027402 | chrX | 153860738 | 153861448 | |
| FAM224A | NR_002161 | chrY | 20488418 | 20492712 | |
| FAM224B | NR_002160 | chrY | 20488439 | 20492736 | |
| FAM226A | NR_026595 | chrX | 72161567 | 72163589 | |
| FAM226B | NR_026594 | chrX | 72161567 | 72163589 | |
| FAM230C | NR_027278 | chrUn_gl000212 | 24048 | 60768 | |
| FAM41AY1 | NR_028083 | chrY | 20551155 | 20566932 | |
| FAM41AY2 | NR_028084 | chrY | 20551155 | 20566932 | |
| FAM46D | NM_001170574 | chrX | 79591002 | 79700810 | protein FAM46D |
| FAM47C | NM_001013736 | chrX | 37026431 | 37029739 | putative protein FAM47C |
| FAM58A | NM_152274 | chrX | 152853382 | 152864632 | cyclin-related protein |
| | | | | | FAM58A isoform 1 |
| FAM9C | NM_174901 | chrX | 13053735 | 13062917 | protein FAM9C |
| FATE1 | NM_033085 | chrX | 150884507 | 150891664 | fetal and adult testis- |
| | | | | | expressed transcript protein |
| FGD1 | NM_004463 | chrX | 54471886 | 54522599 | FYVE, RhoGEF and PH |
| | | | | | domain-containing protein 1 |
| FGF13-AS1 | NR_038405 | chrX | 137794268 | 137798763 | |
| FGF16 | NM_003868 | chrX | 76709646 | 76712013 | fibroblast growth factor 16 |
| FIRRE | NR_026975 | chrX | 130836677 | 130964671 | |
| FLJ43315 | NR_033856 | chrUn_gl000211 | 48502 | 93165 | |
| FLJ43681 | NR_029406 | chr17 | 81174665 | 81188573 | |
| FMR1NB | NM_152578 | chrX | 147062848 | 147108187 | fragile X mental retardation |
| | | | | | 1 neighbor protein |
| FRMD7 | NM_194277 | chrX | 131211020 | 131262050 | FERM domain-containing |
| | | | | | protein 7 |
| FRMD8P1 | NR_033742 | chrX | 64770501 | 64772301 | |
| FRMPD3 | NM_032428 | chrX | 106765679 | 106848474 | FERM and PDZ domain- |
| | | | | | containing protein 3 |
| FRMPD3-AS1 | NR_046750 | chrX | 106756212 | 106789051 | |
| FTH1P18 | NM_001271682 | chrX | 37060954 | 37061867 | ferritin, heavy polypeptide- |
| | | | | | like 18 |
| FTHL17 | NM_031894 | chrX | 31089357 | 31090170 | ferritin heavy polypeptide- |
| | | | | | like 17 |
| GABRQ | NM_018558 | chrX | 151806636 | 151821825 | gamma-aminobutyric acid |
| | | | | | receptor subunit theta |
| | | | | | precursor |
| GAGE12B | NM_001127345 | chrX | 49306370 | 49313636 | G antigen 12B/C/D/E |
| GAGE12F | NM_001098405 | chrX | 49306301 | 49313700 | G antigen 12F |
| GAGE12G | NM_001098409 | chrX | 49335002 | 49342360 | G antigen 12G |
| GAGE12I | NM_001477 | chrX | 49335064 | 49342360 | G antigen 12I |
| GAGE12J | NM_001098406 | chrX | 49178508 | 49294588 | G antigen 12J |
| GAGE13 | NM_001098412 | chrX | 49188080 | 49294588 | G antigen 13 |
| GAGE2B | NM_001098411 | chrX | 49235707 | 49242997 | G antigen 2B/2C |
| GAGE2C | NM_001472 | chrX | 49207148 | 49223953 | G antigen 2B/2C |
| GAGE2D | NM_001098407 | chrX | 49207115 | 49214420 | G antigen 2D |
| GAGE2E | NM_001127200 | chrX | 49207159 | 49214420 | G antigen 2E |
| GAGE4 | NM_001474 | chrX | 49216648 | 49223939 | G antigen 4 |
| GAGE5 | NM_001475 | chrX | 49216656 | 49223943 | G antigen 5 |
| GAGE6 | NM_001476 | chrX | 49325479 | 49332807 | G antigen 6 |
| GAGE7 | NM_021123 | chrX | 49216677 | 49223939 | G antigen 12G |
| GAGE8 | NM_012196 | chrX | 49207159 | 49214420 | G antigen 2D |
| GK | NM_001128127 | chrX | 30671475 | 30749577 | glycerol kinase isoform c |
| GLA | NM_000169 | chrX | 100652778 | 100663001 | alpha-galactosidase A |
| | | | | | precursor |
| GLRA4 | NM_001172285 | chrX | 102973501 | 102983552 | glycine receptor subunit |
| | | | | | alpha-4 isoform 2 precursor |
| GLUD2 | NM_012084 | chrX | 120181461 | 120183796 | glutamate dehydrogenase 2, |
| | | | | | mitochondrial precursor |
| GNL3L | NM_001184819 | chrX | 54556643 | 54593720 | guanine nucleotide-binding |
| | | | | | protein-like 3-like protein |
| GOLGA2P2Y | NR_001555 | chrY | 27601457 | 27606322 | |
| GOLGA2P3Y | NR_002195 | chrY | 27601457 | 27606322 | |
| GPC3 | NM_004484 | chrX | 132669775 | 133119673 | glypican-3 isoform 2 |
| | | | | | precursor |
| GPC4 | NM_001448 | chrX | 132435063 | 132549205 | glypican-4 precursor |
| GPR101 | NM_054021 | chrX | 136112306 | 136113833 | probable G-protein coupled |
| | | | | | receptor 101 |
| GPR112 | NM_153834 | chrX | 135383121 | 135499047 | probable G-protein coupled |
| | | | | | receptor 112 |
| GPR143 | NM_000273 | chrX | 9693452 | 9734005 | G-protein coupled receptor |
| | | | | | 143 |
| GPR174 | NM_032553 | chrX | 78426468 | 78427726 | probable G-protein coupled |
| | | | | | receptor 174 |
| GRPR | NM_005314 | chrX | 16141423 | 16171641 | gastrin-releasing peptide |
| | | | | | receptor |
| GS1-600G8.3 | NR_046087 | chrX | 13328770 | 13338052 | |
| GSPT2 | NM_018094 | chrX | 51486480 | 51489326 | eukaryotic peptide chain |
| | | | | | release factor GTP-binding |
| | | | | | subunit ERF3B |
| GTPBP6 | NM_012227 | chrY | 171416 | 180887 | putative GTP-binding |
| | | | | | protein 6 |
| GUCY2F | NM_001522 | chrX | 108616134 | 108725285 | retinal guanylyl cyclase 2 |
| GYG2P1 | NR_033667 | chrY | 14517914 | 14533389 | |
| HCCS | NM_005333 | chrX | 11129405 | 11141204 | cytochrome c-type heme |
| | | | | | lyase |
| HCFC1 | NM_005334 | chrX | 153213007 | 153236819 | host cell factor 1 |
| HCFC1-AS1 | NR_046608 | chrX | 153234215 | 153235542 | |
| HDAC8 | NM_001166420 | chrX | 71787431 | 71792953 | histone deacetylase 8 |
| | | | | | isoform 4 |
| HDHD1 | NM_001178135 | chrX | 6975626 | 7066231 | pseudouridine-5′- |
| | | | | | monophosphatase isoform c |
| HEPH | NM_001282141 | chrX | 65384071 | 65487230 | hephaestin isoform d |
| | | | | | precursor |
| HLA-DRB3 | NM_022555 | chr6_cox_hap2 | 3934126 | 3947195 | major histocompatibility |
| | | | | | complex, class II, DR beta 3 |
| | | | | | precursor |
| HLA-DRB4 | NM_021983 | chr6_ssto_hap7 | 3850433 | 3865402 | major histocompatibility |
| | | | | | complex, class II, DR beta 4 |
| | | | | | precursor |
| HMGB3 | NM_001301231 | chrX | 150148980 | 150159248 | high mobility group protein |
| | | | | | B3 isoform b |
| HNKNPH2 | NM_019597 | chrX | 100663120 | 100669128 | heterogeneous nuclear |
| | | | | | ribonucleoprotein H2 |
| HPRT1 | NM_000194 | chrX | 133594174 | 133634698 | hypoxanthine-guanine |
| | | | | | phosphoribosyltransferase |
| HS6ST2-AS1 | NR_046691 | chrX | 131801669 | 131803915 | |
| HSD17B10 | NM_004493 | chrX | 53458205 | 53461323 | 3-hydroxyacyl-CoA |
| | | | | | dehydrogenase type-2 |
| | | | | | isoform 1 |
| HTATSF1 | NM_014500 | chrX | 135579670 | 135594503 | HIV Tat-specific factor 1 |
| HYDIN2 | NR_103556 | chr1_gl000192_random | 132568 | 407510 | |
| HYPM | NM_012274 | chrX | 37850069 | 37850570 | huntingtin-interacting |
| | | | | | protein M |
| IDH3G | NM_004135 | chrX | 153051220 | 153059978 | isocitrate dehydrogenase |
| | | | | | [NAD] subunit gamma, |
| | | | | | mitochondrial isoform a |
| | | | | | precursor |
| IGBP1 | NM_001551 | chrX | 69353317 | 69386173 | immunoglobulin-binding |
| | | | | | protein 1 |
| INE1 | NR_024616 | chrX | 47064246 | 47065254 | |
| INE2 | NR_002725 | chrX | 15803838 | 15805712 | |
| IQSEC2 | NM_015075 | chrX | 53262057 | 53310796 | IQ motif and SEC7 domain- |
| | | | | | containing protein 2 isoform |
| | | | | | 2 |
| IRAK1 | NM_001025243 | chrX | 153275956 | 153285342 | interleukin-1 receptor- |
| | | | | | associated kinase 1 isoform |
| | | | | | 3 |
| ITIH6 | NM_198510 | chrX | 54775331 | 54824673 | inter-alpha-trypsin inhibitor |
| | | | | | heavy chain H6 precursor |
| JADE3 | NM_014735 | chrX | 46771867 | 46920641 | protein Jade-3 |
| KANTR | NR_110456 | chrX | 53123338 | 53173249 | |
| KCNE1L | NM_012282 | chrX | 108866928 | 108868393 | potassium voltage-gated |
| | | | | | channel subfamily E |
| | | | | | member 1-like protein |
| KDM5C | NM_001282622 | chrX | 53220502 | 53254604 | lysine-specific demethylase |
| | | | | | 5C isoform 3 |
| KDM6A | NM_001291421 | chrX | 44732420 | 44971857 | lysine-specific demethylase |
| | | | | | 6A isoform 6 |
| KIAA1210 | NM_020721 | chrX | 118212597 | 118284542 | uncharacterized protein |
| | | | | | KIAA1210 |
| KIAA2022 | NM_001008537 | chrX | 73952690 | 74145287 | protein KIAA2022 |
| KIR2DL2 | NM_014219 | chr19_gl000209_random | 21910 | 36449 | killer cell immunoglobulin- |
| | | | | | like receptor 2DL2 precursor |
| KIR2DL5A | NM_020535 | chr19_gl000209_random | 86690 | 96155 | killer cell immunoglobulin- |
| | | | | | like receptor 2DL5A |
| | | | | | precursor |
| KIR2DL5B | NM_001018081 | chr19_gl000209_random | 86745 | 96246 | killer cell immunoglobulin- |
| | | | | | like receptor 2DL5B |
| | | | | | precursor |
| KIR2DS1 | NMJH4512 | chr19_gl000209_random | 115098 | 129113 | killer cell immunoglobulin- |
| | | | | | like receptor 2DS1 precursor |
| KIR2DS2 | NM_001291695 | chr19_gl000209_random | 131432 | 145743 | killer cell immunoglobulin- |
| | | | | | like receptor 2DS2 isoform b |
| | | | | | precursor |
| KIR2DS3 | NM_012313 | chr19_gl000209_random | 98134 | 112667 | killer cell immunoglobulin- |
| | | | | | like receptor 2DS3 precursor |
| KIR2DS5 | NM_014513 | chr19_gl000209_random | 98111 | 113132 | killer cell immunoglobulin- |
| | | | | | like receptor 2DS5 precursor |
| KIR3DS1 | NM_001083539 | chr19_gl000209_random | 70070 | 84658 | killer cell immunoglobulin- |
| | | | | | like receptor 3DS1 isoform 1 |
| | | | | | precursor |
| KLF8 | NM_001159296 | chrX | 56258869 | 56314322 | Krueppel-like factor 8 |
| | | | | | isoform 2 |
| KLHL34 | NM_153270 | chrX | 21673608 | 21676448 | kelch-like protein 34 |
| KRBOX4 | NM_017776 | chrX | 46306623 | 46334074 | KRAB domain-containing |
| | | | | | protein 4 isoform 2 |
| LANCL3 | NM_198511 | chrX | 37430821 | 37536750 | lanC-like protein 3 isoform 1 |
| LAS1L | NM_001170649 | chrX | 64732461 | 64754686 | ribosomal biogenesis protein |
| | | | | | LAS1L isoform 2 |
| LHFPL1 | NM_178175 | chrX | 111873878 | 111923375 | lipoma HMGIC fusion |
| | | | | | partner-like 1 protein |
| | | | | | precursor |
| LINC00087 | NR_024493 | chrX | 134229014 | 134232733 | |
| LINC00266-3 | NR_109817 | chrUn_gl000227 | 66129 | 74245 | |
| LINC00269 | NR_103715 | chrX | 68399399 | 68429767 | |
| LINC00278 | NR_046502 | chrY | 2871036 | 2970313 | |
| LINC00280 | NR_046505 | chrY | 6225259 | 6229454 | |
| LINC00629 | NR_038998 | chrX | 133684053 | 133694428 | |
| LINC00630 | NR_038988 | chrX | 102024094 | 102140338 | |
| LINC00632 | NR_028344 | chrX | 139791923 | 139796996 | |
| LINC00633 | NR_033941 | chrX | 134252881 | 134254405 | |
| LINC00684 | NR_120499 | chrX | 72158002 | 72158798 | |
| LINC00685 | NR_027231 | chrY | 231384 | 232054 | |
| LINC00850 | NR_109813 | chrX | 148958632 | 149008599 | |
| LINC00889 | NR_026935 | chrX | 137696891 | 137699799 | |
| LINC00890 | NR_033974 | chrX | 110754889 | 110765627 | |
| LINC00891 | NR_034005 | chrX | 70917045 | 70923256 | |
| LINC00892 | NR_038461 | chrX | 135721701 | 135724588 | |
| LINC00893 | NR_027455 | chrX | 148609131 | 148621312 | |
| LINC00894 | NR_027456 | chrX | 149106765 | 149185018 | |
| LINC01001 | NR_028326 | chr11 | 126986 | 131920 | |
| LINC01186 | NR_110388 | chrX | 46185358 | 46187109 | |
| LINC01201 | NR_126350 | chrX | 130150442 | 130192120 | |
| LINC01203 | NR_045260 | chrX | 13353359 | 13359944 | |
| LINC01204 | NR_104644 | chrX | 45364632 | 45386484 | |
| LINC01278 | NR_015353 | chrX | 62646438 | 62780873 | |
| LINC01281 | NR_038968 | chrX | 39164209 | 39186616 | |
| LINC01282 | NR_110385 | chrX | 39226538 | 39251028 | |
| LINC01284 | NR_110382 | chrX | 50838681 | 50914232 | |
| LINC01285 | NR_110393 | chrX | 117973518 | 118015977 | |
| LINC01402 | NR_126557 | chrX | 119251551 | 119253610 | |
| LINC01420 | NR_015367 | chrX | 56755717 | 56844004 | |
| LINC01496 | NR_110654 | chrX | 51242760 | 51250293 | |
| LINC01545 | NR_046101 | chrX | 46746853 | 46759139 | |
| LINC01546 | NR_038428 | chrX | 3189860 | 3202694 | |
| LINC01560 | NR_126059 | chrX | 47342114 | 47344626 | |
| LOC100132304 | NR_120493 | chrX | 72158002 | 72158798 | |
| LOC100233156 | NR_037872 | chrUn_gl000218 | 38785 | 97454 | |
| LOC100287728 | NR_103770 | chrX | 134254548 | 134257529 | |
| LOC100288778 | NR_028269 | chr12 | 87983 | 91263 | |
| LOC100288814 | NM_001195081 | chrX | 9935397 | 9936042 | uncharacterized protein |
| | | | | | LOC100288814 |
| LOC100288966 | NM_001257362 | chrUn_gl000213 | 108006 | 139339 | uncharacterized protein |
| | | | | | LOC100288966 |
| LOC100506790 | NR_104652 | chrX | 134530353 | 134531672 | |
| LOC100507412 | NR_038958 | chrUn_gl000220 | 97128 | 126696 | |
| LOC100652931 | NR_104151 | chrY | 24462824 | 24466531 | |
| LOC101927476 | NR_110386 | chrX | 40122169 | 40146974 | |
| LOC101927501 | NR_110387 | chrX | 43036242 | 43085847 | |
| LOC101927830 | NR_109985 | chrX | 154696200 | 154723771 | |
| LOC101928128 | NR_110651 | chrX | 84465711 | 84474295 | |
| LOC101928201 | NR_110390 | chrX | 4545240 | 4551613 | |
| LOC101928259 | NR_110391 | chrX | 71908798 | 71932190 | |
| LOC101928335 | NR_110395 | chrX | 107137826 | 107179210 | |
| LOC101928336 | NR_110396 | chrX | 118425491 | 118469573 | |
| LOC101928358 | NR_110652 | chrX | 107979769 | 107982133 | |
| LOC101928437 | NR_110399 | chrX | 112285954 | 112763885 | |
| LOC101928495 | NR_110409 | chrX | 125243744 | 125249545 | |
| LOC101928564 | NR_104642 | chrX | 36011397 | 36019767 | |
| LOC101929148 | NR_110413 | chrY | 24585086 | 24630861 | |
| LOC102724558 | NR_120328 | chr1_gl000192_random | 429709 | 468683 | |
| LOC104798195 | NR_126564 | chrX | 15621003 | 15639607 | |
| LOC158960 | NR_103768 | chrX | 153652722 | 153656825 | |
| LOC283788 | NR_027436 | chrUn_gl000219 | 56348 | 99642 | |
| LOC389831 | NM_001242480 | chr7_gl000195_random | 42937 | 86719 | uncharacterized protein |
| | | | | | LOC389831 |
| LOC389834 | NR_027420 | chrUn_gl000218 | 46844 | 55049 | |
| LOC389895 | NM_001271560 | chrX | 139173825 | 139175070 | uncharacterized protein |
| | | | | | LOC389895 |
| LOC389906 | NR_034031 | chrX | 3735575 | 3761935 | |
| LOC392452 | NR_102268 | chrX | 45590576 | 45591246 | |
| LOC401585 | NR_125365 | chrX | 45707508 | 45710920 | |
| LOC729609 | NR_024440 | chrX | 20004934 | 20007897 | |
| LONRF3 | NR_110311 | chrX | 118108576 | 118152318 | |
| MAFIP | NR_046442 | chr4_gl000194_random | 61659 | 115073 | |
| MAGEA12 | NM_005367 | chrX | 151899292 | 151903184 | melanoma-associated |
| | | | | | antigen 12 |
| MAGEA2 | NM_005361 | chrX | 151918386 | 151922408 | melanoma-associated |
| | | | | | antigen 1 |
| MAGEA2B | NM_153488 | chrX | 151918403 | 151920099 | melanoma-associated |
| | | | | | antigen 2 |
| MAGEA3 | NM_005362 | chrX | 151934651 | 151938240 | melanoma-associated |
| | | | | | antigen 3 |
| MAGEA6 | NM_175868 | chrX | 151867244 | 151870814 | melanoma-associated |
| | | | | | antigen 6 |
| MAGEA8-AS1 | NR_102703 | chrX | 149007562 | 149025779 | |
| MAGEB1 | NM_002363 | chrX | 30261847 | 30270155 | melanoma-associated |
| | | | | | antigen B1 |
| MAGEB17 | NM_001277307 | chrX | 16185603 | 16189516 | melanoma-associated |
| | | | | | antigen B17 |
| MAGEB18 | NM_173699 | chrX | 26156459 | 26158853 | melanoma-associated |
| | | | | | antigen B18 |
| MAGEB2 | NM_002364 | chrX | 30233674 | 30238206 | melanoma-associated |
| | | | | | antigen B2 |
| MAGEB3 | NM_002365 | chrX | 30248552 | 30255610 | melanoma-associated |
| | | | | | antigen B3 |
| MAGEB4 | NM_002367 | chrX | 30260056 | 30262308 | melanoma-associated |
| | | | | | antigen B4 |
| MAGEB5 | NM_001271752 | chrX | 26234285 | 26236387 | melanoma-associated |
| | | | | | antigen B5 |
| MAGEB6 | NM_173523 | chrX | 26210556 | 26213763 | melanoma-associated |
| | | | | | antigen B6 |
| MAGEC2 | NM_016249 | chrX | 141290127 | 141293076 | melanoma-associated |
| | | | | | antigen C2 |
| MAGED2 | NM_014599 | chrX | 54834770 | 54842448 | melanoma-associated |
| | | | | | antigen D2 |
| MAGEE1 | NM_020932 | chrX | 75648045 | 75651746 | melanoma-associated |
| | | | | | antigen E1 |
| MAGEH1 | NM_014061 | chrX | 55478521 | 55480001 | melanoma-associated |
| | | | | | antigen H1 |
| MAOB | NM_000898 | chrX | 43625856 | 43741721 | amine oxidase [flavin- |
| | | | | | containing] B |
| MAP2K4P1 | NR_029423 | chrX | 72744110 | 72782921 | |
| MAP7D3 | NM_001173517 | chrX | 135295378 | 135333738 | MAP7 domain-containing |
| | | | | | protein 3 isoform 3 |
| MBTPS2 | NM_015884 | chrX | 21857655 | 21903541 | membrane-bound |
| | | | | | transcription factor site-2 |
| | | | | | protease |
| MCTS1 | NM_001137554 | chrX | 119738551 | 119755016 | malignant T-cell-amplified |
| | | | | | sequence 1 isoform 2 |
| MED14OS | NM_001289773 | chrX | 40594647 | 40597953 | uncharacterized protein |
| | | | | | LOC100873985 |
| MGC39584 | NR_038377 | chr4_gl000193_random | 49162 | 88375 | |
| MGC70870 | NR_003682 | chr17_gl000205_random | 116622 | 119732 | |
| MID1IP1 | NM_021242 | chrX | 38660684 | 38665783 | mid1-interacting protein 1 |
| MID1IP1-AS1 | NR_046706 | chrX | 38660500 | 38663136 | |
| MID2 | NM_012216 | chrX | 107069083 | 107174867 | probable E3 ubiquitin- |
| | | | | | protein ligase MID2 isoform |
| | | | | | 1 |
| MIR105-1 | NR_029521 | chrX | 151560690 | 151560771 | |
| MIR105-2 | NR_029522 | chrX | 151562883 | 151562964 | |
| MIR106A | NR_029523 | chrX | 133304227 | 133304308 | |
| MIR1277 | NR_031685 | chrX | 117520356 | 117520434 | |
| MIR1468 | NR_031567 | chrX | 63005881 | 63005967 | |
| MIR188 | NR_029708 | chrX | 49768108 | 49768194 | |
| MIR18B | NR_029949 | chrX | 133304070 | 133304141 | |
| MIR19B2 | NR_029491 | chrX | 133303700 | 133303796 | |
| MIR20B | NR_029950 | chrX | 133303838 | 133303907 | |
| MIR221 | NR_029635 | chrX | 45605584 | 45605694 | |
| MIR222 | NR_029636 | chrX | 45606420 | 45606530 | |
| MIR223 | NR_029637 | chrX | 65238711 | 65238821 | |
| MIR23C | NR_037414 | chrX | 20035205 | 20035305 | |
| MIR325HG | NR_110406 | chrX | 75878198 | 76234957 | |
| MIR362 | NR_029850 | chrX | 49773571 | 49773635 | |
| MIR363 | NR_029852 | chrX | 133303407 | 133303482 | |
| MIR374A | NR_030785 | chrX | 73507120 | 73507192 | |
| MIR374B | NR_030620 | chrX | 73438381 | 73438453 | |
| MIR374C | NR_037511 | chrX | 73438383 | 73438453 | |
| MIR3978 | NR_039774 | chrX | 109325345 | 109325446 | |
| MIR421 | NR_030398 | chrX | 73438211 | 73438296 | |
| MIR424 | NR_029946 | chrX | 133680643 | 133680741 | |
| MIR4328 | NR_036258 | chrX | 78156690 | 78156746 | |
| MIR4329 | NR_036255 | chrX | 112023945 | 112024016 | |
| MIR450A1 | NR_029962 | chrX | 133674370 | 133674461 | |
| MIR450A2 | NR_030227 | chrX | 133674537 | 133674637 | |
| MIR450B | NR_030587 | chrX | 133674214 | 133674292 | |
| MIR4536-1 | NR_039764 | chrX | 55477892 | 55477953 | |
| MIR4767 | NR_039924 | chrX | 7065900 | 7065978 | |
| MIR4769 | NR_039926 | chrX | 47446827 | 47446904 | |
| MIR500A | NR_030224 | chrX | 49773038 | 49773122 | |
| MIR500B | NR_036257 | chrX | 49775279 | 49775358 | |
| MIR501 | NR_030225 | chrX | 49774329 | 49774413 | |
| MIR502 | NR_030226 | chrX | 49779205 | 49779291 | |
| MIR503 | NR_030228 | chrX | 133680357 | 133680428 | |
| MIR503HG | NR_024607 | chrX | 133677406 | 133680660 | |
| MIR505 | NR_030230 | chrX | 139006306 | 139006390 | |
| MIR514A1 | NR_030238 | chrX | 146360764 | 146360862 | |
| MIR514A2 | NR_030239 | chrX | 146366158 | 146366246 | |
| MIR514A3 | NR_030240 | chrX | 146366158 | 146366246 | |
| MIR532 | NR_030241 | chrX | 49767753 | 49767844 | |
| MIR542 | NR_030399 | chrX | 133675370 | 133675467 | |
| MIR545 | NR_030258 | chrX | 73506938 | 73507044 | |
| MIR6086 | NR_106734 | chrX | 13608410 | 13608465 | |
| MIR6089 | NR_106737 | chrY | 2477231 | 2477295 | |
| MIR6134 | NR_106750 | chrX | 28513671 | 28513780 | |
| MIR660 | NR_030397 | chrX | 49777848 | 49777945 | |
| MIR664B | NR_049842 | chrX | 153996870 | 153996931 | |
| MIR6724-1 | NR_106782 | chrUn_gl000220 | 148703 | 148795 | |
| MIR6724-2 | NR_128715 | chrUn_gl000220 | 148703 | 148795 | |
| MIR6724-3 | NR_128716 | chrUn_gl000220 | 148703 | 148795 | |
| MIR6724-4 | NR_128717 | chrUn_gl000220 | 148703 | 148795 | |
| MIR676 | NR_037494 | chrX | 69242706 | 69242773 | |
| MIR6857 | NR_106916 | chrX | 53432604 | 53432697 | |
| MIR6858 | NR_106917 | chrX | 153678667 | 153678734 | |
| MIR6894 | NR_106954 | chrX | 53228070 | 53228127 | |
| MIR6895 | NR_106955 | chrX | 53224592 | 53224670 | |
| MIR718 | NR_031757 | chrX | 153285370 | 153285440 | |
| MIR766 | NR_030413 | chrX | 118780700 | 118780811 | |
| MIR767 | NR_030409 | chrX | 151561892 | 151562001 | |
| MIR8088 | NR_107055 | chrX | 52079698 | 52079784 | |
| MIR888 | NR_030592 | chrX | 145076301 | 145076378 | |
| MIR890 | NR_030589 | chrX | 145075792 | 145075869 | |
| MIR891A | NR_030581 | chrX | 145109311 | 145109390 | |
| MIR891B | NR_030590 | chrX | 145082570 | 145082649 | |
| MIR892A | NR_030584 | chrX | 145078186 | 145078261 | |
| MIR892B | NR_030593 | chrX | 145078715 | 145078792 | |
| MIR892C | NR_106783 | chrX | 145074267 | 145074344 | |
| MIR92A2 | NR_029509 | chrX | 133303567 | 133303642 | |
| MIR934 | NR_030631 | chrX | 135633036 | 135633119 | |
| MIR98 | NR_029513 | chrX | 53583183 | 53583302 | |
| MIRLET7F2 | NR_029484 | chrX | 53584152 | 53584235 | |
| MORF4L2 | NM_001142424 | chrX | 102930425 | 102941746 | mortality factor 4-like |
| | | | | | protein 2 |
| MORF4L2- | NR_038978 | chrX | 102942211 | 102947484 | |
| AS1 | | | | | |
| MOSPD1 | NM_019556 | chrX | 134021661 | 134049297 | motile sperm domain- |
| | | | | | containing protein 1 |
| MPC1L | NM_001195522 | chrX | 40482817 | 40483391 | mitochondrial pyruvate |
| | | | | | carrier 1-like protein |
| MSN | NM_002444 | chrX | 64887510 | 64961793 | moesin |
| MTMR8 | NM_017677 | chrX | 63487960 | 63615333 | myotubularin-related protein |
| | | | | | 8 |
| MTRNR2L10 | NM_001190708 | chrX | 55207823 | 55208944 | humanin-like 10 |
| MXRA5 | NM_015419 | chrX | 3226608 | 3264684 | matrix-remodeling- |
| | | | | | associated protein 5 |
| | | | | | precursor |
| NAA10 | NM_001256120 | chrX | 153195279 | 153200607 | N-alpha-acetyltransferase 10 |
| | | | | | isoform 3 |
| NAP1L2 | NM_021963 | chrX | 72432136 | 72434710 | nucleosome assembly |
| | | | | | protein 1-like 2 |
| NAP1L3 | NM_004538 | chrX | 92925924 | 92928682 | nucleosome assembly |
| | | | | | protein 1-like 3 |
| NAP1L6 | NR_027291 | chrX | 72345875 | 72347919 | |
| NDP | NM_000266 | chrX | 43808023 | 43832921 | norrin precursor |
| NDUFA1 | NM_004541 | chrX | 119005733 | 119010629 | NADH dehydrogenase |
| | | | | | [ubiquinone] 1 alpha |
| | | | | | subcomplex subunit 1 |
| NDUFB11 | NM_001135998 | chrX | 47001614 | 47004609 | NADH dehydrogenase |
| | | | | | [ubiquinone] 1 beta |
| | | | | | subcomplex subunit 11, |
| | | | | | mitochondrial isoform 2 |
| NGFRAP1 | NM_014380 | chrX | 102632108 | 102633092 | protein BEX3 isoform b |
| NHS-AS1 | NR_046632 | chrX | 17570469 | 17577248 | |
| NKAPP1 | NR_027131 | chrX | 119370308 | 119379122 | |
| NKRF | NM_001173488 | chrX | 118722299 | 118727113 | NF-kappa-B-repressing |
| | | | | | factor isoform 2 |
| NLGN4Y-AS1 | NR_046504 | chrY | 16905521 | 16915913 | |
| NOX1 | NM_007052 | chrX | 100098312 | 100129334 | NADPH oxidase 1 isoform 1 |
| NUDT10 | NM_153183 | chrX | 51075082 | 51080377 | diphosphoinositol |
| | | | | | polyphosphate |
| | | | | | phosphohydrolase 3-alpha |
| NXF2 | NM_022053 | chrX | 101615315 | 101694929 | nuclear RNA export factor 2 |
| NXF2B | NM_001099686 | chrX | 101615315 | 101694929 | nuclear RNA export factor 2 |
| NXF3 | NM_022052 | chrX | 102330749 | 102348022 | nuclear RNA export factor 3 |
| NXF4 | NR_002216 | chrX | 101804892 | 101826621 | |
| NXT2 | NM_001242618 | chrX | 108780346 | 108787927 | NTF2-related export protein |
| | | | | | 2 isoform 3 |
| OCRL | NM_001587 | chrX | 128674251 | 128726530 | inositol polyphosphate 5- |
| | | | | | phosphatase OCRL-1 |
| | | | | | isoform b |
| OTC | NM_000531 | chrX | 38211735 | 38280703 | ornithine |
| | | | | | carbamoyltransferase, |
| | | | | | mitochondrial precursor |
| OTUD6A | NM_207320 | chrX | 69282340 | 69284029 | OTU domain-containing |
| | | | | | protein 6A |
| P2RY10 | NM_014499 | chrX | 78200828 | 78217438 | putative P2Y purinoceptor |
| | | | | | 10 |
| PABPC1L2B- | NR_110398 | chrX | 72300005 | 72304474 | |
| AS1 | | | | | |
| PABPC5-AS1 | NR_110659 | chrX | 90669901 | 90689998 | |
| PAGE1 | NM_003785 | chrX | 49452053 | 49460596 | P antigen family member 1 |
| PAGE3 | NR_033460 | chrX | 55284848 | 55291165 | |
| PAGE4 | NM_007003 | chrX | 49593905 | 49598637 | P antigen family member 4 |
| PAGE5 | NM_130467 | chrX | 55246790 | 55250541 | P antigen family member 5 |
| | | | | | isoform 1 |
| PAK3 | NM_001128166 | chrX | 110187512 | 110464173 | serine/threonine-protein |
| | | | | | kinase PAK 3 isoform a |
| PBDC1 | NM_001300888 | chrX | 75392763 | 75398145 | protein PBDC1 isoform 2 |
| PCYT1B | NM_004845 | chrX | 24576203 | 24665455 | choline-phosphate |
| | | | | | cytidylyltransferase B |
| | | | | | isoform 1 |
| PCYT1B-AS1 | NR_046638 | chrX | 24668189 | 24676354 | |
| PDK3 | NM_001142386 | chrX | 24483343 | 24568583 | pyruvate dehydrogenase |
| | | | | | kinase, isozyme 3 isoform 1 |
| | | | | | precursor |
| PDZD11 | NM_016484 | chrX | 69506210 | 69509798 | PDZ domain-containing |
| | | | | | protein 11 |
| PGAM4 | NM_001029891 | chrX | 77223457 | 77225135 | phosphoglycerate mutase 4 |
| PGRMC1 | NM_001282621 | chrX | 118370207 | 118378429 | membrane-associated |
| | | | | | progesterone receptor |
| | | | | | component 1 isoform 2 |
| PHEX-AS1 | NR_046639 | chrX | 22180848 | 22191100 | |
| PHKA1 | NM_001172436 | chrX | 71798663 | 71934029 | phosphorylase b kinase |
| | | | | | regulatory subunit alpha, |
| | | | | | skeletal muscle isoform |
| | | | | | isoform 3 |
| PHKA2-AS1 | NR_029379 | chrX | 18908413 | 18913093 | |
| PIH1D3 | NM_173494 | chrX | 106449861 | 106487473 | protein PIH1D3 |
| PLCXD1 | NM_018390 | chrY | 148060 | 170022 | PI-PLC X domain- |
| | | | | | containing protein 1 |
| PLP1 | NM_001128834 | chrX | 103031438 | 103047547 | myelin proteolipid protein |
| | | | | | isoform 1 |
| PLS3 | NM_001282337 | chrX | 114795176 | 114885179 | plastin-3 isoform 3 |
| PLS3-AS1 | NR_110383 | chrX | 114752496 | 114797058 | |
| PLXNB3 | NM_005393 | chrX | 153029650 | 153044801 | plexin-B3 isoform 1 |
| | | | | | precursor |
| PNCK | NM_001135740 | chrX | 152935187 | 152938743 | calcium/calmodulin- |
| | | | | | dependent protein kinase |
| | | | | | type 1B isoform b |
| PNMA3 | NM_013364 | chrX | 152224765 | 152228827 | paraneoplastic antigen Ma3 |
| | | | | | isoform 1 |
| PPEF1-AS1 | NR_046642 | chrX | 18706762 | 18710806 | |
| PRKX-AS1 | NR_046643 | chrX | 3577527 | 3586231 | |
| PRKY | NR_028062 | chrY | 7142012 | 7249588 | |
| PRORY | NM_001282471 | chrY | 23544859 | 23548246 | proline-rich protein, Y- |
| | | | | | linked |
| PRPS1 | NM_001204402 | chrX | 106871653 | 106894256 | ribose-phosphate |
| | | | | | pyrophosphokinase 1 |
| | | | | | isoform 2 |
| PRR32 | NM_001122716 | chrX | 125953746 | 125955768 | proline-rich protein 32 |
| PRRG1 | NM_001173489 | chrX | 37208582 | 37316548 | transmembrane gamma- |
| | | | | | carboxyglutamic acid |
| | | | | | protein 1 isoform 1 |
| | | | | | precursor |
| PRRG3 | NM_024082 | chrX | 150863729 | 150870063 | transmembrane gamma- |
| | | | | | carboxyglutamic acid |
| | | | | | protein 3 precursor |
| PRY | NM_004676 | chrY | 24217902 | 24242154 | PTPN13-like protein, Y- |
| | | | | | linked |
| PRY2 | NM_001002758 | chrY | 24217902 | 24242154 | PTPN13-like protein, Y- |
| | | | | | linked |
| PSMD10 | NM_170750 | chrX | 107327434 | 107334874 | 26S proteasome non-ATPase |
| | | | | | regulatory subunit 10 |
| | | | | | isoform 2 |
| PTCHD1-AS | NR_073010 | chrX | 22277913 | 23311263 | |
| RAB40A | NM_080879 | chrX | 102754680 | 102774417 | ras-related protein Rab-40A |
| RAB40AL | NM_001031834 | chrX | 102192199 | 102193228 | ras-related protein Rab-40A- |
| | | | | | like |
| RAB9B | NM_016370 | chrX | 103077254 | 103087212 | ras-related protein Rab-9B |
| RAI2 | NM_001172743 | chrX | 17818168 | 17879457 | retinoic acid-induced protein |
| | | | | | 2 isoform 1 |
| RAP2C | NM_001271187 | chrX | 131337051 | 131353508 | ras-related protein Rap-2c |
| | | | | | isoform 2 |
| RAP2C-AS1 | NR_110410 | chrX | 131352534 | 131566839 | |
| RBMX | NM_002139 | chrX | 135955605 | 135962939 | RNA-binding motif protein, |
| | | | | | X chromosome isoform 1 |
| RBMY1A3P | NR_001547 | chrY | 9154669 | 9160483 | |
| RBMY2EP | NR_001574 | chrY | 23557033 | 23563448 | |
| RENBP | NM_002910 | chrX | 153200721 | 153210232 | N-acylglucosamine 2- |
| | | | | | epimerase |
| REPS2 | NM_001080975 | chrX | 16964813 | 17171403 | ralBP1-associated Eps |
| | | | | | domain-containing protein 2 |
| | | | | | isoform 2 |
| RGAG1 | NM_020769 | chrX | 109662284 | 109699562 | retrotransposon gag domain- |
| | | | | | containing protein 1 |
| RGAG4 | NM_001024455 | chrX | 71346960 | 71351751 | retrotransposon gag domain- |
| | | | | | containing protein 4 |
| RGN | NM_001282848 | chrX | 46937753 | 46952713 | regucalcin isoform 2 |
| RIBC1 | NM_144968 | chrX | 53449804 | 53456776 | RIB43A-like with coiled- |
| | | | | | coils protein 1 isoform 2 |
| RNA45S5 | NR_046235 | chrUn_gl000220 | 105423 | 118780 | |
| RNA5-8S5 | NR_003285 | chrUn_gl000220 | 155996 | 156152 | |
| RNF113A | NM_006978 | chrX | 119004494 | 119005791 | RING finger protein 113A |
| RP11-87M18.2 | NR_110412 | chrX | 36383740 | 36458375 | |
| RP2 | NM_006915 | chrX | 46696346 | 46741791 | protein XRP2 |
| RPL36A | NM_001199972 | chrX | 100645877 | 100648840 | 60S ribosomal protein L36a |
| | | | | | isoform b |
| RPL36A- | NM_001199973 | chrX | 100645877 | 100669128 | RPL36A-HNRNPH2 protein |
| HNRNPH2 | | | | | isoform a |
| RPL39 | NM_001000 | chrX | 118920466 | 118925622 | 60S ribosomal protein L39 |
| RPS26P11 | NR_002309 | chrX | 71264258 | 71264811 | |
| RPS4X | NM_001007 | chrX | 71492452 | 71497141 | 40S ribosomal protein S4, X |
| | | | | | isoform X isoform |
| RPS4Y1 | NM_001008 | chrY | 2709622 | 2734997 | 40S ribosomal protein S4, Y |
| | | | | | isoform 1 |
| RRAGB | NM_006064 | chrX | 55744109 | 55785207 | ras-related GTP-binding |
| | | | | | protein B short isoform |
| S100G | NM_004057 | chrX | 16668280 | 16672791 | protein S100-G |
| SATL1 | NM_001012980 | chrX | 84347291 | 84363974 | spermidine/spermine N(1)- |
| | | | | | acetyltransferase-like protein |
| | | | | | 1 |
| SCARNA9L | NR_023358 | chrX | 20154183 | 20154531 | |
| SCGB1C2 | NM_001097610 | chr11 | 193079 | 194500 | secretoglobin family 1C |
| | | | | | member 2 precursor |
| SCML1 | NM_001037536 | chrX | 17755568 | 17773108 | sex comb on midleg-like |
| | | | | | protein 1 isoform c |
| SEPT6 | NM_015129 | chrX | 118750908 | 118827333 | septin-6 isoform B |
| SH2D1A | NM_001114937 | chrX | 123480131 | 123507010 | SH2 domain-containing |
| | | | | | protein 1A isoform 2 |
| SH3BGRL | NM_003022 | chrX | 80457302 | 80554046 | SH3 domain-binding |
| | | | | | glutamic acid-rich-like |
| | | | | | protein |
| SLC25A5 | NM_001152 | chrX | 118602362 | 118605359 | ADP/ATP translocase 2 |
| SLC25A5-AS1 | NR_028443 | chrX | 118599995 | 118603083 | |
| SLC25A53 | NM_001012755 | chrX | 103343897 | 103401708 | solute carrier family 25 |
| | | | | | member 53 |
| SLC9A6 | NM_001042537 | chrX | 135067585 | 135129428 | sodium/hydrogen exchanger |
| | | | | | 6 isoform a precursor |
| SLITRK2 | NM_001144009 | chrX | 144902865 | 144907360 | SLIT and NTRK-like protein |
| | | | | | 2 precursor |
| SLITRK4 | NM_173078 | chrX | 142710594 | 142723019 | SLIT and NTRK-like protein |
| | | | | | 4 precursor |
| SMC1A | NM_006306 | chrX | 53401069 | 53449677 | structural maintenance of |
| | | | | | chromosomes protein 1A |
| | | | | | isoform 1 |
| SMIM10 | NM_001163438 | chrX | 134124967 | 134126503 | small integral membrane |
| | | | | | protein 10 |
| SMIM9 | NM_001162936 | chrX | 154051622 | 154062937 | small integral membrane |
| | | | | | protein 9 precursor |
| SMPX | NM_014332 | chrX | 21724089 | 21776278 | small muscular protein |
| SNORA11 | NR_002953 | chrX | 54840802 | 54840933 | |
| SNORA11C | NR_003710 | chrX | 47248048 | 47248175 | |
| SNORA36A | NR_002969 | chrX | 153996802 | 153996932 | |
| SNORA56 | NR_002984 | chrX | 154003272 | 154003401 | |
| SNORA69 | NR_002584 | chrX | 118921315 | 118921447 | |
| SNORD61 | NR_002735 | chrX | 135961357 | 135961430 | |
| SOWAHD | NM_001105576 | chrX | 118892575 | 118894165 | ankyrin repeat domain- |
| | | | | | containing protein |
| | | | | | SOWAHD |
| SOX3 | NM_005634 | chrX | 139585151 | 139587225 | transcription factor SOX-3 |
| SPANXN2 | NM_001009615 | chrX | 142795134 | 142803762 | sperm protein associated |
| | | | | | with the nucleus on the X |
| | | | | | chromosome N2 |
| SPANXN4 | NM_001009613 | chrX | 142113703 | 142122066 | sperm protein associated |
| | | | | | with the nucleus on the X |
| | | | | | chromosome N4 |
| SPIN3 | NM_001010862 | chrX | 57017263 | 57021988 | spindlin-3 |
| SPIN4 | NM_001012968 | chrX | 62567106 | 62571218 | spindlin-4 |
| SPRY3 | NM_005840 | chrY | 59100456 | 59115123 | protein sprouty homolog 3 |
| SRPK3 | NM_001170761 | chrX | 153046455 | 153051187 | SRSF protein kinase 3 |
| | | | | | isoform 3 |
| SRPX2 | NM_014467 | chrX | 99899162 | 99926296 | sushi repeat-containing |
| | | | | | protein SRPX2 precursor |
| SRY | NM_003140 | chrY | 2654895 | 2655782 | sex-determining region Y |
| | | | | | protein |
| SSR4 | NM_001204526 | chrX | 153059903 | 153063967 | translocon-associated protein |
| | | | | | subunit delta isoform 1 |
| | | | | | precursor |
| SSX9 | NR_073393 | chrX | 48160984 | 48165614 | |
| STK26 | NM_016542 | chrX | 131157244 | 131209971 | serine/threonine-protein |
| | | | | | kinase 26 isoform 1 |
| SUPT20HL1 | NM_001136234 | chrX | 24380877 | 24383541 | transcription factor SPT20 |
| | | | | | homolog-like 1 |
| SUPT20HL2 | NM_001136233 | chrX | 24328978 | 24331432 | putative transcription factor |
| | | | | | SPT20 homolog-like 2 |
| SYAP1 | NR_033181 | chrX | 16737706 | 16780807 | |
| SYN1 | NM_133499 | chrX | 47431299 | 47479256 | synapsin-1 isoform Ib |
| SYP-AS1 | NR_046649 | chrX | 49055297 | 49058913 | |
| TAB3 | NM_152787 | chrX | 30845558 | 30907511 | TGF-beta-activated kinase 1 |
| | | | | | and MAP3K7-binding |
| | | | | | protein 3 |
| TBL1Y | NM_134259 | chrY | 6778726 | 6959724 | F-box-like/WD repeat- |
| | | | | | containing protein TBL1Y |
| TCEAL1 | NM_001006640 | chrX | 102883647 | 102885876 | transcription elongation |
| | | | | | factor A protein-like 1 |
| TCEAL2 | NM_080390 | chrX | 101380659 | 101382684 | transcription elongation |
| | | | | | factor A protein-like 2 |
| TCEAL3 | NM_001006933 | chrX | 102862833 | 102864855 | transcription elongation |
| | | | | | factor A protein-like 3 |
| TCEAL4 | NM_001300901 | chrX | 102831158 | 102842664 | transcription elongation |
| | | | | | factor A protein-like 4 |
| | | | | | isoform 5 |
| TCEAL5 | NM_001012979 | chrX | 102528617 | 102531797 | transcription elongation |
| | | | | | factor A protein-like 5 |
| TCEAL6 | NM_001006938 | chrX | 101394932 | 101397388 | transcription elongation |
| | | | | | factor A protein-like 6 |
| TCEAL7 | NM_152278 | chrX | 102585113 | 102587251 | transcription elongation |
| | | | | | factor A protein-like 7 |
| TCEAL8 | NM_001006684 | chrX | 102507922 | 102510121 | transcription elongation |
| | | | | | factor A protein-like 8 |
| TCEANC | NM_001297564 | chrX | 13671224 | 13683527 | transcription elongation |
| | | | | | factor A N-terminal and |
| | | | | | central domain-containing |
| | | | | | protein isoform 2 |
| TCP11X2 | NM_001277423 | chrX | 101715239 | 101726732 | T-complex protein 11 |
| | | | | | homolog |
| TDGF1P3 | NR_002718 | chrX | 109763539 | 109766249 | |
| TENM1 | NM_001163279 | chrX | 123509755 | 124097666 | teneurin-1 isoform 2 |
| TEX13A | NM_031274 | chrX | 104463610 | 104465377 | testis-expressed sequence |
| | | | | | 13A protein |
| TFDP3 | NM_016521 | chrX | 132350696 | 132352376 | transcription factor Dp |
| | | | | | family member 3 |
| TGIF2LY | NM_139214 | chrY | 3447125 | 3448082 | homeobox protein TGIF2LY |
| THOC2 | NM_001081550 | chrX | 122734411 | 122866904 | THO complex subunit 2 |
| TIMP1 | NM_003254 | chrX | 47441689 | 47446190 | metalloproteinase inhibitor 1 |
| | | | | | precursor |
| TLR7 | NM_016562 | chrX | 12885201 | 12908480 | toll-like receptor 7 precursor |
| TLR8 | NM_138636 | chrX | 12924738 | 12941288 | toll-like receptor 8 isoform 2 |
| | | | | | precursor |
| TLR8-AS1 | NR_030727 | chrX | 12920935 | 12961419 | |
| TMEM164 | NM_017698 | chrX | 109245862 | 109421016 | transmembrane protein 164 |
| | | | | | isoform a precursor |
| TMEM255A | NM_017938 | chrX | 119392504 | 119445391 | transmembrane protein |
| | | | | | 255A isoform 1 |
| TMEM257 | NM_004709 | chrX | 144908927 | 144911370 | transmembrane protein 257 |
| TMEM27 | NM_020665 | chrX | 15645438 | 15683154 | collectrin precursor |
| TMEM31 | NM_182541 | chrX | 102965836 | 102968960 | transmembrane protein 31 |
| TMLHE-AS1 | NR_039991 | chrX | 154696200 | 154723771 | |
| TMSB15A | NM_021992 | chrX | 101768609 | 101771699 | thymosin beta-15A |
| TMSB4Y | NM_004202 | chrY | 15815446 | 15817902 | thymosin beta-4, Y- |
| | | | | | chromosomal |
| TNMD | NM_022144 | chrX | 99839789 | 99854882 | tenomodulin |
| TREX2 | NM_080701 | chrX | 152710177 | 152711945 | three prime repair |
| | | | | | exonuclease 2 |
| TRO | NR_073148 | chrX | 54946995 | 54957866 | |
| TRPC5OS | NM_001195578 | chrX | 111119427 | 111147213 | putative uncharacterized |
| | | | | | protein TRPC5OS |
| TSC22D3 | NM_004089 | chrX | 106956451 | 106960291 | TSC22 domain family |
| | | | | | protein 3 isoform 2 |
| TSIX | NR_003255 | chrX | 73012039 | 73049066 | |
| TSPAN6 | NM_001278742 | chrX | 99882104 | 99892101 | tetraspanin-6 isoform c |
| | | | | | precursor |
| TSPY10 | NM_001282469 | chrY | 9365507 | 9368122 | testis-specific Y-encoded |
| | | | | | protein 10 |
| TSPYL2 | NM_022117 | chrX | 53111541 | 53117728 | testis-specific Y-encoded- |
| | | | | | like protein 2 |
| TSR2 | NM_058163 | chrX | 54466852 | 54471731 | pre-rRNA-processing |
| | | | | | protein TSR2 homolog |
| TTTY1 | NR_001538 | chrY | 9590764 | 9611898 | |
| TTTY11 | NR_001548 | chrY | 8651358 | 8685423 | |
| TTTY12 | NR_001551 | chrY | 7672964 | 7678723 | |
| TTTY15 | NR_001545 | chrY | 14774297 | 14804153 | |
| TTTY16 | NR_001552 | chrY | 7567397 | 7569288 | |
| TTTY18 | NR_001550 | chrY | 8551410 | 8551919 | |
| TTTY19 | NR_001549 | chrY | 8572512 | 8573324 | |
| TTTY1B | NR_003589 | chrY | 9590764 | 9611928 | |
| TTTY2 | NR_001536 | chrY | 9573894 | 9596085 | |
| TTTY20 | NR_001546 | chrY | 9167488 | 9172441 | |
| TTTY21 | NR_001535 | chrY | 9555261 | 9558905 | |
| TTTY21B | NR_003588 | chrY | 9555261 | 9558905 | |
| TTTY22 | NR_001539 | chrY | 9638761 | 9650854 | |
| TTTY2B | NR_003590 | chrY | 9573894 | 9596085 | |
| TTTY3 | NR_001524 | chrY | 27874636 | 27879535 | |
| TTTY3B | NR_002176 | chrY | 27874636 | 27879535 | |
| TTTY6 | NR_001527 | chrY | 24585739 | 24587606 | |
| TTTY6B | NR_002175 | chrY | 24585736 | 24587584 | |
| TTTY7 | NR_001534 | chrY | 9544432 | 9552871 | |
| TTTY7B | NR_003592 | chrY | 9544432 | 9552871 | |
| TTTY8 | NR_001533 | chrY | 9528708 | 9531308 | |
| TTTY8B | NR_003591 | chrY | 9528708 | 9531308 | |
| TTTY9A | NR_001530 | chrY | 20891767 | 20901083 | |
| TTTY9B | NR_002159 | chrY | 20891767 | 20901083 | |
| TXLNG | NM_018360 | chrX | 16804554 | 16862642 | gamma-taxilin isoform 1 |
| TXLNGY | NR_045129 | chrY | 21729243 | 21752309 | |
| UBA1 | NM_153280 | chrX | 47050198 | 47074527 | ubiquitin-like modifier- |
| | | | | | activating enzyme 1 |
| UBE2A | NM_003336 | chrX | 118708429 | 118718392 | ubiquitin-conjugating |
| | | | | | enzyme E2 A isoform 1 |
| UBE2DNL | NR_024062 | chrX | 84189156 | 84189896 | |
| UBE2E4P | NR_110506 | chrX | 14262386 | 14263545 | |
| UPF3B | NM_023010 | chrX | 118967988 | 118986991 | regulator of nonsense |
| | | | | | transcripts 3B isoform 2 |
| UQCRBP1 | NR_002308 | chrX | 56763220 | 56764017 | |
| USP11 | NM_004651 | chrX | 47092313 | 47107727 | ubiquitin carboxyl-terminal |
| | | | | | hydrolase 11 |
| USP26 | NM_031907 | chrX | 132159506 | 132162300 | ubiquitin carboxyl-terminal |
| | | | | | hydrolase 26 |
| USP27X | NM_001145073 | chrX | 49644469 | 49647168 | ubiquitin carboxyl-terminal |
| | | | | | hydrolase 27 |
| USP27X-AS1 | NR_026742 | chrX | 49641326 | 49643959 | |
| USP9Y | NM_004654 | chrY | 14813159 | 14972768 | probable ubiquitin carboxyl- |
| | | | | | terminal hydrolase FAF-Y |
| UTY | NR_047602 | chrY | 15360258 | 15592550 | |
| UXT | NM_153477 | chrX | 47511190 | 47518579 | protein UXT isoform 1 |
| UXT-AS1 | NR_028119 | chrX | 47518231 | 47519510 | |
| VGLL1 | NM_016267 | chrX | 135614310 | 135638966 | transcription cofactor |
| | | | | | vestigial-like protein 1 |
| VMA21 | NM_001017980 | chrX | 150565656 | 150577836 | vacuolar ATPase assembly |
| | | | | | integral membrane protein |
| | | | | | VMA21 |
| VSIG1 | NM_182607 | chrX | 107288199 | 107322414 | V-set and immunoglobulin |
| | | | | | domain-containing protein 1 |
| | | | | | isoform 2 precursor |
| VSIG4 | NM_001184830 | chrX | 65241579 | 65259967 | V-set and immunoglobulin |
| | | | | | domain-containing protein 4 |
| | | | | | isoform 4 precursor |
| WBP5 | NM_016303 | chrX | 102611379 | 102613397 | WW domain-binding protein |
| | | | | | 5 |
| WNK3 | NM_001002838 | chrX | 54219255 | 54384438 | serine/threonine-protein |
| | | | | | kinase WNK3 isoform 2 |
| XAGE2 | NM_130777 | chrX | 52380347 | 52387021 | X antigen family member 2 |
| XAGE3 | NM_130776 | chrX | 52891557 | 52896332 | X antigen family member 3 |
| XAGE5 | NM_130775 | chrX | 52841227 | 52847322 | X antigen family member 5 |
| XGY2 | NR_003254 | chrY | 2620336 | 2643037 | |
| XIAP | NR_037916 | chrX | 122994016 | 123047829 | |
| XIST | NR_001564 | chrX | 73040485 | 73072588 | |
| XK | NM_021083 | chrX | 37545132 | 37591383 | membrane transport protein |
| | | | | | XK precursor |
| XKRX | NM_212559 | chrX | 100168430 | 100183898 | XK-related protein 2 |
| XKRY | NM_004677 | chrY | 20297334 | 20298915 | testis-specific XK-related |
| | | | | | protein, Y-linked 2 |
| XKRY2 | NM_001002906 | chrY | 20297334 | 20298915 | testis-specific XK-related |
| | | | | | protein, Y-linked 2 |
| XRCC6P5 | NR_024608 | chrX | 98716599 | 99194841 | |
| YIPF6 | NM_173834 | chrX | 67718623 | 67757127 | protein YIPF6 isoform A |
| YY2 | NM_206923 | chrX | 21874104 | 21876845 | transcription factor YY2 |
| ZBTB33 | NM_001184742 | chrX | 119384609 | 119392251 | transcriptional regulator |
| | | | | | Kaiso |
| ZC3H12B | NM_001010888 | chrX | 64708614 | 64727767 | probable ribonuclease |
| | | | | | ZC3H12B |
| ZC4H2 | NM_001178033 | chrX | 64135681 | 64196413 | zinc finger C4H2 domain- |
| | | | | | containing protein isoform 3 |
| ZCCHC13 | NM_203303 | chrX | 73524024 | 73524869 | zinc finger CCHC domain- |
| | | | | | containing protein 13 |
| ZFP92 | NM_001136273 | chrX | 152683780 | 152687086 | zinc finger protein 92 |
| | | | | | homolog |
| ZFX-AS1 | NR_046657 | chrX | 24164341 | 24167771 | |
| ZFY | NM_001145276 | chrY | 2803111 | 2850547 | zinc finger Y-chromosomal |
| | | | | | protein isoform 3 |
| ZMAT1 | NM_001282400 | chrX | 101137259 | 101187039 | zinc finger matrin-type |
| | | | | | protein 1 isoform 4 |
| ZNF157 | NM_003446 | chrX | 47229998 | 47273098 | zinc finger protein 157 |
| ZNF275 | NM_001080485 | chrX | 152599612 | 152618384 | zinc finger protein 275 |
| ZNF41 | NM_007130 | chrX | 47305560 | 47342345 | zinc finger protein 41 |
| ZNF630-AS1 | NR_046742 | chrX | 47915698 | 47925970 | |
| ZNF674 | NM_001146291 | chrX | 46357159 | 46404892 | zinc finger protein 674 |
| | | | | | isoform 2 |
| ZNF674-AS1 | NR_015378 | chrX | 46404924 | 46407910 | |
| ZNF711 | NM_021998 | chrX | 84498996 | 84528368 | zinc finger protein 711 |
| ZNF81 | NM_007137 | chrX | 47696300 | 47781655 | zinc finger protein 81 |
| ZRSR2 | NM_005089 | chrX | 15808573 | 15841382 | U2 small nuclear |
| | | | | | ribonucleoprotein auxiliary |
| | | | | | factor 35 kDa subunit- |
| | | | | | related protein 2 |

TABLE 11
The candidate reference loci for use with tissue samples collected
from healthy subjects, patients with myocardial infarction, and
cancer-unaffected tissues of cancer patients.

| Symbol | Refseq | Chr | Start | End | Description |
|---|---|---|---|---|---|
| DDX11L16 | NR_110561 | chrY | 59358328 | 59360854 | |
| LINC00685 | NR_027231 | chrY | 231384 | 232054 | |
| MIR6089 | NR_106737 | chrY | 2477231 | 2477295 | |

TABLE 12
The genes, whose CN can be measured using Human Breast Cancer Copy Number
PCR Array kit (Qiagen)

| Symbol | Refseq | Chr | Start | End | Description |
|---|---|---|---|---|---|
| AKT1 | NM_001014431 | chr14 | 105235686 | 105262080 | RAC-alpha |
| | | | | | serine/threonine- |
| | | | | | protein kinase |
| AURKA | NM_198437 | chr20 | 54944444 | 54967351 | aurora kinase A |
| BCHE | NM_000055 | chr3 | 165490691 | 165555253 | cholinesterase |
| | | | | | precursor |
| BCL2L1 | NM_001191 | chr20 | 30252260 | 30310656 | bcl-2-like protein 1 |
| | | | | | isoform 2 |
| C11orf30 | NM_001300944 | chr11 | 76156068 | 76263943 | protein EMSY |
| | | | | | isoform 3 |
| CCND1 | NM_053056 | chr11 | 69455872 | 69469242 | G1/S-specific |
| | | | | | cyclin-D1 |
| CDK4 | NM_000075 | chr12 | 58141509 | 58146230 | cyclin-dependent |
| | | | | | kinase 4 |
| CDKN2A | NM_058197 | chr9 | 21967750 | 21974826 | cyclin-dependent |
| | | | | | kinase inhibitor 2A |
| | | | | | isoform p12 |
| CSMD1 | NM_033225 | chr8 | 2792874 | 4852328 | CUB and sushi |
| | | | | | domain-containing |
| | | | | | protein 1 precursor |
| EGFR | NM_201283 | chr7 | 55086724 | 55224644 | epidermal growth |
| | | | | | factor receptor |
| | | | | | isoform c precursor |
| ERBB2 | NM_004448 | chr17 | 37856230 | 37884915 | receptor tyrosine- |
| | | | | | protein kinase erbB- |
| | | | | | 2 isoform a |
| | | | | | precursor |
| FGFR1 | NM_023106 | chr8 | 38268655 | 38326352 | fibroblast growth |
| | | | | | factor receptor 1 |
| | | | | | isoform 4 precursor |
| FGFR2 | NM_001144919 | chr10 | 123241366 | 123357972 | fibroblast growth |
| | | | | | factor receptor 2 |
| | | | | | isoform 9 precursor |
| MTDH | NM_178812 | chr8 | 98656406 | 98742488 | protein LYRIC |
| MYC | NM_002467 | chr8 | 128748314 | 128753680 | myc proto- |
| | | | | | oncogene protein |
| NCOA3 | NM_001174088 | chr20 | 46130600 | 46285621 | nuclear receptor |
| | | | | | coactivator 3 |
| | | | | | isoform d |
| PAK1 | NM_002576 | chr11 | 77033059 | 77185108 | serine/threonine- |
| | | | | | protein kinase PAK |
| | | | | | 1 isoform 2 |
| PPAPDC1B | NM_001102560 | chr8 | 38124497 | 38126738 | phosphatidate |
| | | | | | phosphatase |
| | | | | | PPAPDC1B |
| | | | | | isoform 3 |
| PTEN | NM_000314 | chr10 | 89623194 | 89728532 | phosphatidylinositol |
| | | | | | 3,4,5-trisphosphate |
| | | | | | 3-phosphatase and |
| | | | | | dual-specificity |
| | | | | | protein phosphatase |
| | | | | | PTEN |
| PTK2 | NM_001199649 | chr8 | 141668480 | 142011412 | focal adhesion |
| | | | | | kinase 1 isoform c |
| RB1 | NM_000321 | chr13 | 48877882 | 49056026 | retinoblastoma- |
| | | | | | associated protein |
| TFDP1 | NR_026580 | chr13 | 114239002 | 114295788 | |
| TOP2A | NM_001067 | chr17 | 38544772 | 38574202 | DNA |
| | | | | | topoisomerase 2- |
| | | | | | alpha |

TABLE 13
Genes with high expression and CN-invariant in the TCGA EOC samples.

| Symbol | Refseq | Chr | Start | End | Description |
|---|---|---|---|---|---|
| ABCB4 | NM_018849 | chr7 | 87031360 | 87105019 | multidrug resistance |
| | | | | | protein 3 isoform B |
| ABHD5 | NM_016006 | chr3 | 43732374 | 43764217 | 1-acylglycerol-3- |
| | | | | | phosphate O- |
| | | | | | acyltransferase ABHD5 |
| ACYP2 | NM_138448 | chr2 | 54342409 | 54532435 | acylphosphatase-2 |
| AFF3 | NM_001025108 | chr2 | 100163715 | 100722045 | AF4/FMR2 family |
| | | | | | member 3 isoform 2 |
| AGAP1 | NM_001244888 | chr2 | 236402732 | 236761846 | arf-GAP with GTPase, |
| | | | | | ANK repeat and PH |
| | | | | | domain-containing |
| | | | | | protein 1 isoform 3 |
| AMD1 | NM_001287216 | chr6 | 111195986 | 111216915 | S-adenosylmethionine |
| | | | | | decarboxylase |
| | | | | | proenzyme isoform 5 |
| ANK2 | NM_001127493 | chr4 | 113739238 | 114304896 | ankyrin-2 isoform 3 |
| ARSE | NM_001282628 | chrX | 2852672 | 2882494 | arylsulfatase E isoform 1 |
| ASAP1 | NM_018482 | chr8 | 131064350 | 131455906 | arf-GAP with SH3 |
| | | | | | domain, ANK repeat |
| | | | | | and PH domain- |
| | | | | | containing protein 1 |
| | | | | | isoform 1 |
| ASCC3 | NM_001284271 | chr6 | 101163006 | 101329248 | activating signal |
| | | | | | cointegrator 1 complex |
| | | | | | subunit 3 isoform c |
| ATAD2B | NM_001242338 | chr2 | 23971533 | 24149984 | ATPase family AAA |
| | | | | | domain-containing |
| | | | | | protein 2B isoform 2 |
| ATF7IP2 | NM_024997 | chr16 | 10479911 | 10577495 | activating transcription |
| | | | | | factor 7-interacting |
| | | | | | protein 2 isoform 1 |
| ATXN7 | NM_001128149 | chr3 | 63953419 | 63989136 | ataxin-7 isoform c |
| AUTS2 | NM_015570 | chr7 | 69063904 | 70258054 | autism susceptibility |
| | | | | | gene 2 protein isoform 1 |
| BATF3 | NM_018664 | chr1 | 212859758 | 212873327 | basic leucine zipper |
| | | | | | transcriptional factor |
| | | | | | ATF-like 3 |
| BMPR2 | NM_001204 | chr2 | 203241049 | 203432474 | bone morphogenetic |
| | | | | | protein receptor type-2 |
| | | | | | precursor |
| BTNL8 | NM_001159707 | chr5 | 180326076 | 180377906 | butyrophilin-like protein |
| | | | | | 8 isoform 3 precursor |
| C1orf21 | NM_030806 | chr1 | 184356149 | 184598155 | uncharacterized protein |
| | | | | | C1orf21 |
| CACNB2 | NM_201571 | chr10 | 18429741 | 18830688 | voltage-dependent L- |
| | | | | | type calcium channel |
| | | | | | subunit beta-2 isoform 6 |
| CAMTA1 | NR_038934 | chr1 | 6845383 | 6948261 | |
| CASC5 | NM_170589 | chr15 | 40886446 | 40954881 | protein CASC5 isoform 1 |
| CASQ2 | NM_001232 | chr1 | 116242625 | 116311426 | calsequestrin-2 |
| | | | | | precursor |
| CCDC88A | NM_018084 | chr2 | 55514977 | 55647057 | girdin isoform 2 |
| CHL1 | NR_045572 | chr3 | 239325 | 290282 | |
| CHST15 | NM_014863 | chr10 | 125779168 | 125851940 | carbohydrate |
| | | | | | sulfotransferase 15 |
| | | | | | isoform 2 |
| CLASP1 | NM_001142273 | chr2 | 122095351 | 122407052 | CLIP-associating |
| | | | | | protein 1 isoform 2 |
| CLIC4 | NM_013943 | chr1 | 25071759 | 25170815 | chloride intracellular |
| | | | | | channel protein 4 |
| CLMN | NM_024734 | chr14 | 95648275 | 95786245 | calmin |
| COPA | NM_001098398 | chr1 | 160258376 | 160313354 | coatomer subunit alpha |
| | | | | | isoform 1 |
| CUL3 | NM_001257197 | chr2 | 225334866 | 225450114 | cullin-3 isoform 2 |
| DAB1 | NM_021080 | chr1 | 57463578 | 58716211 | disabled homolog 1 |
| DAPK1 | NM_001288729 | chr9 | 90113449 | 90323549 | death-associated protein |
| | | | | | kinase 1 |
| DDAH1 | NM_012137 | chr1 | 85784167 | 85930889 | N(G),N(G)- |
| | | | | | dimethylarginine |
| | | | | | dimethylaminohydrolase |
| | | | | | 1 isoform 1 |
| DEGS1 | NM_003676 | chr1 | 224370909 | 224381142 | sphingolipid delta(4)- |
| | | | | | desaturase DES1 |
| DEPDC1 | NM_001114120 | chr1 | 68939834 | 68962904 | DEP domain-containing |
| | | | | | protein 1A isoform a |
| DNM3 | NM_015569 | chr1 | 171810617 | 172381857 | dynamin-3 isoform a |
| DPPA4 | NM_018189 | chr3 | 109044987 | 109056419 | developmental |
| | | | | | pluripotency-associated |
| | | | | | protein 4 |
| DYRK1A | NM_001396 | chr21 | 38792601 | 38887679 | dual specificity tyrosine- |
| | | | | | phosphorylation- |
| | | | | | regulated kinase 1A |
| | | | | | isoform 1 |
| EFHC2 | NM_025184 | chrX | 44007127 | 44202923 | EF-hand domain- |
| | | | | | containing family |
| | | | | | member C2 |
| EHBP1 | NM_015252 | chr2 | 62933000 | 63273621 | EH domain-binding |
| | | | | | protein 1 isoform 1 |
| EHD3 | NM_014600 | chr2 | 31456879 | 31491260 | EH domain-containing |
| | | | | | protein 3 |
| EIF5 | NM_001969 | chr14 | 103800338 | 103811361 | eukaryotic translation |
| | | | | | initiation factor 5 |
| ENPP2 | NR_045555 | chr8 | 120569316 | 120605248 | |
| EPB41 | NM_001166007 | chr1 | 29213602 | 29446558 | protein 4.1 isoform 5 |
| EPHB2 | NM_004442 | chr1 | 23037330 | 23241823 | ephrin type-B receptor 2 |
| | | | | | isoform 2 precursor |
| ERBB4 | NM_005235 | chr2 | 212240441 | 213403352 | receptor tyrosine-protein |
| | | | | | kinase erbB-4 isoform |
| | | | | | JM-a/CVT-1 precursor |
| ERC2 | NM_015576 | chr3 | 55542335 | 56502391 | ERC protein 2 |
| ESRRG | NM_206594 | chr1 | 216676587 | 217262987 | estrogen-related |
| | | | | | receptor gamma isoform 2 |
| FAHD2A | NM_016044 | chr2 | 96068447 | 96078879 | fumarylacetoacetate |
| | | | | | hydrolase domain- |
| | | | | | containing protein 2A |
| FAM49A | NM_030797 | chr2 | 16730729 | 16847134 | protein FAM49A |
| FAT1 | NM_005245 | chr4 | 187508936 | 187644987 | protocadherin Fat 1 |
| | | | | | precursor |
| FCGR2A | NM_001136219 | chr1 | 161475204 | 161489360 | low affinity |
| | | | | | immunoglobulin gamma |
| | | | | | Fc region receptor II-a |
| | | | | | isoform 1 precursor |
| FGF12 | NM_004113 | chr3 | 191857181 | 192445388 | fibroblast growth factor |
| | | | | | 12 isoform 2 |
| FGGY | NM_001113411 | chr1 | 59762624 | 60228402 | FGGY carbohydrate |
| | | | | | kinase domain- |
| | | | | | containing protein |
| | | | | | isoform a |
| FHIT | NM_002012 | chr3 | 59735035 | 61237133 | bis(5′-adenosyl)- |
| | | | | | triphosphatase |
| FHL1 | NM_001159702 | chrX | 135229558 | 135293518 | four and a half LIM |
| | | | | | domains protein 1 |
| | | | | | isoform 1 |
| FHL2 | NM_201557 | chr2 | 105977282 | 106055230 | four and a half LIM |
| | | | | | domains protein 2 |
| FUT9 | NM_006581 | chr6 | 96463844 | 96663488 | alpha-(1,3)- |
| | | | | | fucosyltransferase 9 |
| GAP43 | NM_002045 | chr3 | 115342150 | 115440334 | neuromodulin isoform 2 |
| GBE1 | NM_000158 | chr3 | 81538849 | 81810950 | 1,4-alpha-glucan- |
| | | | | | branching enzyme |
| GLI2 | NM_005270 | chr2 | 121554866 | 121750229 | zinc finger protein GLI2 |
| GOLIM4 | NM_014498 | chr3 | 167727653 | 167813417 | Golgi integral |
| | | | | | membrane protein 4 |
| GPBP1L1 | NM_021639 | chr1 | 46092975 | 46152302 | vasculin-like protein 1 |
| GRM8 | NM_001127323 | chr7 | 126078651 | 126892428 | metabotropic glutamate |
| | | | | | receptor 8 isoform b |
| | | | | | precursor |
| GTF2F2 | NM_004128 | chr13 | 45694630 | 45858239 | general transcription |
| | | | | | factor IIF subunit 2 |
| H6PD | NM_001282587 | chr1 | 9299902 | 9331394 | GDH/6PGL |
| | | | | | endoplasmic |
| | | | | | bifunctional protein |
| | | | | | isoform 1 precursor |
| HHAT | NM_001122834 | chr1 | 210501595 | 210849638 | protein-cysteine N- |
| | | | | | palmitoyltransferase |
| | | | | | HHAT isoform 1 |
| HS3ST1 | NM_005114 | chr4 | 11399987 | 11430537 | heparan sulfate |
| | | | | | glucosamine 3-O- |
| | | | | | sulfotransferase 1 |
| | | | | | precursor |
| HTR4 | NM_199453 | chr5 | 147830594 | 148016624 | 5-hydroxytryptamine |
| | | | | | receptor 4 isoform g |
| HYAL3 | NM_003549 | chr3 | 50330258 | 50336899 | hyaluronidase-3 isoform |
| | | | | | 1 precursor |
| IL15 | NR_037840 | chr4 | 142557748 | 142655140 | |
| IL5RA | NM_175726 | chr3 | 3108007 | 3152058 | interleukin-5 receptor |
| | | | | | subunit alpha isoform 1 |
| | | | | | precursor |
| KCNAB1 | NM_172159 | chr3 | 156008775 | 156256927 | voltage-gated potassium |
| | | | | | channel subunit beta-1 |
| | | | | | isoform 3 |
| LAMC3 | NM_006059 | chr9 | 133884503 | 133968446 | laminin subunit gamma- |
| | | | | | 3 precursor |
| LDB2 | NM_001290 | chr4 | 16503164 | 16900424 | LIM domain-binding |
| | | | | | protein 2 isoform a |
| LEF1 | NM_001130714 | chr4 | 108968700 | 109090112 | lymphoid enhancer- |
| | | | | | binding factor 1 isoform 3 |
| LPHN3 | NM_015236 | chr4 | 62362838 | 62938168 | latrophilin-3 precursor |
| LRCH1 | NM_015116 | chr13 | 47127295 | 47319036 | leucine-rich repeat and |
| | | | | | calponin homology |
| | | | | | domain-containing |
| | | | | | protein 1 isoform 2 |
| LRP1B | NM_018557 | chr2 | 140988995 | 142889270 | low-density lipoprotein |
| | | | | | receptor-related protein |
| | | | | | 1B precursor |
| LYST | NM_001301365 | chr1 | 235824330 | 236047008 | lysosomal-trafficking |
| | | | | | regulator |
| MAN1A1 | NM_005907 | chr6 | 119498365 | 119670931 | mannosyl- |
| | | | | | oligosaccharide 1,2- |
| | | | | | alpha-mannosidase IA |
| MCTP1 | NM_001002796 | chr5 | 94041241 | 94417570 | multiple C2 and |
| | | | | | transmembrane domain- |
| | | | | | containing protein 1 |
| | | | | | isoform S |
| MFAP3L | NM_021647 | chr4 | 170907747 | 170947581 | microfibrillar-associated |
| | | | | | protein 3-like isoform 1 |
| | | | | | precursor |
| MORC3 | NM_015358 | chr21 | 37692486 | 37748944 | MORC family CW-type |
| | | | | | zinc finger protein 3 |
| MTA1 | NM_001203258 | chr14 | 105886185 | 105937057 | metastasis-associated |
| | | | | | protein MTA1 isoform |
| | | | | | MTA1s |
| NECAP2 | NM_001145278 | chr1 | 16767166 | 16786584 | adaptin ear-binding |
| | | | | | coat-associated protein 2 |
| | | | | | isoform 3 |
| NEIL3 | NM_018248 | chr4 | 178230990 | 178284092 | endonuclease 8-like 3 |
| NLGN4X | NM_181332 | chrX | 5808066 | 6146923 | neuroligin-4, X-linked |
| NMD3 | NM_015938 | chr3 | 160939098 | 160969795 | 60S ribosomal export |
| | | | | | protein NMD3 |
| NOTCH2 | NM_024408 | chr1 | 120454175 | 120612317 | neurogenic locus notch |
| | | | | | homolog protein 2 |
| | | | | | isoform 1 preproprotein |
| NRP2 | NM_018534 | chr2 | 206547223 | 206641880 | neuropilin-2 isoform 4 |
| | | | | | precursor |
| NRXN1 | NM_004801 | chr2 | 50145642 | 51259674 | neurexin-1-beta isoform |
| | | | | | alpha1 precursor |
| NT5C2 | NM_001134373 | chr10 | 104847773 | 104953063 | cytosolic purine 5′- |
| | | | | | nucleotidase |
| NTNG1 | NM_014917 | chr1 | 107682744 | 108024475 | netrin-G1 isoform 3 |
| | | | | | precursor |
| NUP133 | NM_018230 | chr1 | 229577043 | 229644088 | nuclear pore complex |
| | | | | | protein Nup133 |
| PARN | NM_001134477 | chr16 | 14529556 | 14724128 | poly(A)-specific |
| | | | | | ribonuclease PARN |
| | | | | | isoform 2 |
| PCDH7 | NM_032456 | chr4 | 30722029 | 30726957 | protocadherin-7 isoform |
| | | | | | b precursor |
| PCOLCE2 | NM_013363 | chr3 | 142536701 | 142608045 | procollagen C- |
| | | | | | endopeptidase enhancer |
| | | | | | 2 precursor |
| PDE2A | NM_001146209 | chr11 | 72287183 | 72380108 | cGMP-dependent 3′,5′- |
| | | | | | cyclic |
| | | | | | phosphodiesterase |
| | | | | | isoform PDE2A4 |
| PDE6C | NM_006204 | chr10 | 95372344 | 95425429 | cone cGMP-specific |
| | | | | | 3′,5′-cyclic |
| | | | | | phosphodiesterase |
| | | | | | subunit alpha′ |
| PDIA3 | NM_005313 | chr15 | 44038589 | 44064804 | protein disulfide- |
| | | | | | isomerase A3 precursor |
| PDZK1 | NM_001201325 | chr1 | 145727665 | 145764206 | Na(+)/H(+) exchange |
| | | | | | regulatory cofactor |
| | | | | | NHE-RF3 isoform 1 |
| PHTF1 | NM_006608 | chr1 | 114239823 | 114301777 | putative homeodomain |
| | | | | | transcription factor 1 |
| PLEKHA2 | NM_021623 | chr8 | 38758752 | 38831430 | pleckstrin homology |
| | | | | | domain-containing |
| | | | | | family A member 2 |
| POU2F1 | NM_001198783 | chr1 | 167298280 | 167396582 | POU domain, class 2, |
| | | | | | transcription factor 1 |
| | | | | | isoform 2 |
| PRDM16 | NM_022114 | chr1 | 2985741 | 3355185 | PR domain zinc finger |
| | | | | | protein 16 isoform 1 |
| PRDM5 | NM_001300824 | chr4 | 121613067 | 121844021 | PR domain zinc finger |
| | | | | | protein 5 isfoorm 3 |
| PRKCE | NM_005400 | chr2 | 45879042 | 46415129 | protein kinase C epsilon |
| | | | | | type |
| PRKCZ | NM_001033582 | chr1 | 2036154 | 2116834 | protein kinase C zeta |
| | | | | | type isoform 2 |
| PRUNE | NM_021222 | chr1 | 150980972 | 151008189 | protein prune homolog |
| | | | | | isoform 1 |
| PTGS2 | NM_000963 | chr1 | 186640943 | 186649559 | prostaglandin G/H |
| | | | | | synthase 2 precursor |
| PTPRF | NM_130440 | chr1 | 43996546 | 44089343 | receptor-type tyrosine- |
| | | | | | protein phosphatase F |
| | | | | | isoform 2 precursor |
| PTPRZ1 | NM_002851 | chr7 | 121513158 | 121702090 | receptor-type tyrosine- |
| | | | | | protein phosphatase zeta |
| | | | | | isoform 1 precursor |
| PUM1 | NM_014676 | chr1 | 31404352 | 31538564 | pumilio homolog 1 |
| | | | | | isoform 2 |
| RAD52 | NM_001297419 | chr12 | 1020901 | 1099207 | DNA repair protein |
| | | | | | RAD52 homolog |
| | | | | | isoform a |
| RAI2 | NM_001172743 | chrX | 17818168 | 17879457 | retinoic acid-induced |
| | | | | | protein 2 isoform 1 |
| RNF144A | NM_014746 | chr2 | 7057522 | 7184309 | E3 ubiquitin-protein |
| | | | | | ligase RNF144A |
| SCHIP1 | NM_014575 | chr3 | 158991035 | 159615155 | schwannomin- |
| | | | | | interacting protein 1 |
| | | | | | isoform 1 |
| SERTAD2 | NM_014755 | chr2 | 64858754 | 64881046 | SERTA domain- |
| | | | | | containing protein 2 |
| SLC12A6 | NM_001042495 | chr15 | 34522196 | 34630265 | solute carrier family 12 |
| | | | | | member 6 isoform c |
| SLC15A2 | NM_001145998 | chr3 | 121613170 | 121663034 | solute carrier family 15 |
| | | | | | member 2 isoform b |
| SLC4A4 | NM_003759 | chr4 | 72204769 | 72437804 | electrogenic sodium |
| | | | | | bicarbonate |
| | | | | | cotransporter 1 isoform 2 |
| SMYD3 | NM_022743 | chr1 | 245912641 | 246580714 | histone-lysine N- |
| | | | | | methyltransferase |
| | | | | | SMYD3 isoform 2 |
| SNTG2 | NM_018968 | chr2 | 946553 | 1371384 | gamma-2-syntrophin |
| SPATS2L | NM_001100424 | chr2 | 201170984 | 201346986 | SPATS2-like protein |
| | | | | | isoform b |
| TBL1X | NM_001139468 | chrX | 9431334 | 9687780 | F-box-like/WD repeat- |
| | | | | | containing protein |
| | | | | | TBL1X isoform b |
| TGFBR3 | NM_001195683 | chr1 | 92145899 | 92351836 | transforming growth |
| | | | | | factor beta receptor type |
| | | | | | 3 isoform b precursor |
| THRAP3 | NM_005119 | chr1 | 36690016 | 36770957 | thyroid hormone |
| | | | | | receptor-associated |
| | | | | | protein 3 |
| TIAM1 | NM_003253 | chr21 | 32490735 | 32931290 | T-lymphoma invasion |
| | | | | | and metastasis-inducing |
| | | | | | protein 1 |
| TLE4 | NM_007005 | chr9 | 82186687 | 82341796 | transducin-like enhancer |
| | | | | | protein 4 isoform 3 |
| TNIK | NM_001161561 | chr3 | 170780291 | 171178197 | TRAF2 and NCK- |
| | | | | | interacting protein |
| | | | | | kinase isoform 3 |
| TRIM48 | NM_024114 | chr11 | 55029657 | 55038595 | tripartite motif- |
| | | | | | containing protein 48 |
| TRPM8 | NM_024080 | chr2 | 234826042 | 234928166 | transient receptor |
| | | | | | potential cation channel |
| | | | | | subfamily M member 8 |
| TSPAN9 | NM_001168320 | chr12 | 3186520 | 3395730 | tetraspanin-9 |
| TTF1 | NM_001205296 | chr9 | 135250936 | 135282238 | transcription termination |
| | | | | | factor 1 isoform 2 |
| VPS8 | NM_015303 | chr3 | 184529930 | 184770402 | vacuolar protein sorting- |
| | | | | | associated protein 8 |
| | | | | | homolog isoform b |
| WASF3 | NM_001291965 | chr13 | 27131839 | 27263082 | wiskott-Aldrich |
| | | | | | syndrome protein family |
| | | | | | member 3 isoform 2 |
| WBSCR16 | NM_001281441 | chr7 | 74470621 | 74489717 | Williams-Beuren |
| | | | | | syndrome chromosomal |
| | | | | | region 16 protein |
| | | | | | isoform 3 |
| WDFY3 | NM_014991 | chr4 | 85590692 | 85887544 | WD repeat and FYVE |
| | | | | | domain-containing |
| | | | | | protein 3 |
| WISP1 | NM_080838 | chr8 | 134203281 | 134243932 | WNT1-inducible- |
| | | | | | signaling pathway |
| | | | | | protein 1 isoform 2 |
| | | | | | precursor |
| XRCC5 | NM_021141 | chr2 | 216974019 | 217071016 | X-ray repair cross- |
| | | | | | complementing protein 5 |
| YEATS2 | NM_018023 | chr3 | 183415605 | 183530413 | YEATS domain- |
| | | | | | containing protein 2 |
| ZNF274 | NM_133502 | chr19 | 58694355 | 58724928 | neurotrophin receptor- |
| | | | | | interacting factor |
| | | | | | homolog isoform c |
| ZNF702P | NR_003578 | chr19 | 53471503 | 53496784 | |

TABLE 14
Genes with high expression in GSE9899 and CN-invariant in TCGA EOC samples

| Symbol | Refseq | Chr | Start | End | Description |
|---|---|---|---|---|---|
| ABCB4 | NM_018849 | chr7 | 87031360 | 87105019 | multidrug resistance |
| | | | | | protein 3 isoform B |
| ABHD5 | NM_016006 | chr3 | 43732374 | 43764217 | 1-acylglycerol-3- |
| | | | | | phosphate O- |
| | | | | | acyltransferase ABHD5 |
| ACYP2 | NM_138448 | chr2 | 54342409 | 54532435 | acylphosphatase-2 |
| AFF3 | NM_001025108 | chr2 | 100163715 | 100722045 | AF4/FMR2 family |
| | | | | | member 3 isoform 2 |
| AGAP1 | NM_001244888 | chr2 | 236402732 | 236761846 | arf-GAP with GTPase, |
| | | | | | ANK repeat and PH |
| | | | | | domain-containing |
| | | | | | protein 1 isoform 3 |
| AMD1 | NM_001287216 | chr6 | 111195986 | 111216915 | S-adenosylmethionine |
| | | | | | decarboxylase |
| | | | | | proenzyme isoform 5 |
| ANK2 | NM_001127493 | chr4 | 113739238 | 114304896 | ankyrin-2 isoform 3 |
| ARSE | NM_001282628 | chrX | 2852672 | 2882494 | arylsulfatase E isoform 1 |
| ASAP1 | NM_018482 | chr8 | 131064350 | 131455906 | arf-GAP with SH3 |
| | | | | | domain, ANK repeat |
| | | | | | and PH domain- |
| | | | | | containing protein 1 |
| | | | | | isoform 1 |
| ASCC3 | NM_001284271 | chr6 | 101163006 | 101329248 | activating signal |
| | | | | | cointegrator 1 complex |
| | | | | | subunit 3 isoform c |
| ATAD2B | NM_001242338 | chr2 | 23971533 | 24149984 | ATPase family AAA |
| | | | | | domain-containing |
| | | | | | protein 2B isoform 2 |
| ATF7IP2 | NM_024997 | chr16 | 10479911 | 10577495 | activating transcription |
| | | | | | factor 7-interacting |
| | | | | | protein 2 isoform 1 |
| ATXN7 | NM_001128149 | chr3 | 63953419 | 63989136 | ataxin-7 isoform c |
| AUTS2 | NM_015570 | chr7 | 69063904 | 70258054 | autism susceptibility |
| | | | | | gene 2 protein isoform 1 |
| BATF3 | NM_018664 | chr1 | 212859758 | 212873327 | basic leucine zipper |
| | | | | | transcriptional factor |
| | | | | | ATF-like 3 |
| BMPR2 | NM_001204 | chr2 | 203241049 | 203432474 | bone morphogenetic |
| | | | | | protein receptor type-2 |
| | | | | | precursor |
| BTNL8 | NM_001159707 | chr5 | 180326076 | 180377906 | butyrophilin-like protein |
| | | | | | 8 isoform 3 precursor |
| C1orf21 | NM_030806 | chr1 | 184356149 | 184598155 | uncharacterized protein |
| | | | | | C1orf21 |
| CACNB2 | NM_201571 | chr10 | 18429741 | 18830688 | voltage-dependent L- |
| | | | | | type calcium channel |
| | | | | | subunit beta-2 isoform 6 |
| CAMTA1 | NR_038934 | chr1 | 6845383 | 6948261 | |
| CASC5 | NM_170589 | chr15 | 40886446 | 40954881 | protein CASC5 isoform 1 |
| CASQ2 | NM_001232 | chr1 | 116242625 | 116311426 | calsequestrin-2 |
| | | | | | precursor |
| CCDC88A | NM_018084 | chr2 | 55514977 | 55647057 | girdin isoform 2 |
| CHL1 | NR_045572 | chr3 | 239325 | 290282 | |
| CHST15 | NM_014863 | chr10 | 125779168 | 125851940 | carbohydrate |
| | | | | | sulfotransferase 15 |
| | | | | | isoform 2 |
| CLASP1 | NM_001142273 | chr2 | 122095351 | 122407052 | CLIP-associating |
| | | | | | protein 1 isoform 2 |
| CLIC4 | NM_013943 | chr1 | 25071759 | 25170815 | chloride intracellular |
| | | | | | channel protein 4 |
| CLMN | NM_024734 | chr14 | 95648275 | 95786245 | calmin |
| COPA | NM_001098398 | chr1 | 160258376 | 160313354 | coatomer subunit alpha |
| | | | | | isoform 1 |
| CUL3 | NM_001257197 | chr2 | 225334866 | 225450114 | cullin-3 isoform 2 |
| DAB1 | NM_021080 | chr1 | 57463578 | 58716211 | disabled homolog 1 |
| DAPK1 | NM_001288729 | chr9 | 90113449 | 90323549 | death-associated protein |
| | | | | | kinase 1 |
| DDAH1 | NM_012137 | chr1 | 85784167 | 85930889 | N(G),N(G)- |
| | | | | | dimethylarginine |
| | | | | | dimethylaminohydrolase |
| | | | | | 1 isoform 1 |
| DEGS1 | NM_003676 | chr1 | 224370909 | 224381142 | sphingolipid delta(4)- |
| | | | | | desaturase DES1 |
| DEPDC1 | NM_001114120 | chr1 | 68939834 | 68962904 | DEP domain-containing |
| | | | | | protein 1A isoform a |
| DNM3 | NM_015569 | chr1 | 171810617 | 172381857 | dynamin-3 isoform a |
| DPPA4 | NM_018189 | chr3 | 109044987 | 109056419 | developmental |
| | | | | | pluripotency-associated |
| | | | | | protein 4 |
| DYRK1A | NM_001396 | chr21 | 38792601 | 38887679 | dual specificity tyrosine- |
| | | | | | phosphorylation- |
| | | | | | regulated kinase 1A |
| | | | | | isoform 1 |
| EFHC2 | NM_025184 | chrX | 44007127 | 44202923 | EF-hand domain- |
| | | | | | containing family |
| | | | | | member C2 |
| EHBP1 | NM_015252 | chr2 | 62933000 | 63273621 | EH domain-binding |
| | | | | | protein 1 isoform 1 |
| EHD3 | NM_014600 | chr2 | 31456879 | 31491260 | EH domain-containing |
| | | | | | protein 3 |
| EIF5 | NM_001969 | chr14 | 103800338 | 103811361 | eukaryotic translation |
| | | | | | initiation factor 5 |
| ENPP2 | NR_045555 | chr8 | 120569316 | 120605248 | |
| EPB41 | NM_001166007 | chr1 | 29213602 | 29446558 | protein 4.1 isoform 5 |
| EPHB2 | NM_004442 | chr1 | 23037330 | 23241823 | ephrin type-B receptor 2 |
| | | | | | isoform 2 precursor |
| ERBB4 | NM_005235 | chr2 | 212240441 | 213403352 | receptor tyrosine-protein |
| | | | | | kinase erbB-4 isoform |
| | | | | | JM-a/CVT-1 precursor |
| ERC2 | NM_015576 | chr3 | 55542335 | 56502391 | ERC protein 2 |
| ESRRG | NM_206594 | chr1 | 216676587 | 217262987 | estrogen-related |
| | | | | | receptor gamma isoform 2 |
| FAHD2A | NM_016044 | chr2 | 96068447 | 96078879 | fumarylacetoacetate |
| | | | | | hydrolase domain- |
| | | | | | containing protein 2A |
| FAM49A | NM_030797 | chr2 | 16730729 | 16847134 | protein FAM49A |
| FAT1 | NM_005245 | chr4 | 187508936 | 187644987 | protocadherin Fat 1 |
| | | | | | precursor |
| FCGR2A | NM_001136219 | chr1 | 161475204 | 161489360 | low affinity |
| | | | | | immunoglobulin gamma |
| | | | | | Fc region receptor II-a |
| | | | | | isoform 1 precursor |
| FGF12 | NM_004113 | chr3 | 191857181 | 192445388 | fibroblast growth factor |
| | | | | | 12 isoform 2 |
| FGGY | NM_001113411 | chr1 | 59762624 | 60228402 | FGGY carbohydrate |
| | | | | | kinase domain- |
| | | | | | containing protein |
| | | | | | isoform a |
| FHIT | NM_002012 | chr3 | 59735035 | 61237133 | bis(5′-adenosyl)- |
| | | | | | triphosphatase |
| FHL1 | NM_001159702 | chrX | 135229558 | 135293518 | four and a half LIM |
| | | | | | domains protein 1 |
| | | | | | isoform 1 |
| FHL2 | NM_201557 | chr2 | 105977282 | 106055230 | four and a half LIM |
| | | | | | domains protein 2 |
| FUT9 | NM_006581 | chr6 | 96463844 | 96663488 | alpha-(1,3)- |
| | | | | | fucosyltransferase 9 |
| GAP43 | NM_002045 | chr3 | 115342150 | 115440334 | neuromodulin isoform 2 |
| GBE1 | NM_000158 | chr3 | 81538849 | 81810950 | 1,4-alpha-glucan- |
| | | | | | branching enzyme |
| GLI2 | NM_005270 | chr2 | 121554866 | 121750229 | zinc finger protein GLI2 |
| GOLIM4 | NM_014498 | chr3 | 167727653 | 167813417 | Golgi integral |
| | | | | | membrane protein 4 |
| GPBP1L1 | NM_021639 | chr1 | 46092975 | 46152302 | vasculin-like protein 1 |
| GRM8 | NM_001127323 | chr7 | 126078651 | 126892428 | metabotropic glutamate |
| | | | | | receptor 8 isoform b |
| | | | | | precursor |
| GTF2F2 | NM_004128 | chr13 | 45694630 | 45858239 | general transcription |
| | | | | | factor IIF subunit 2 |
| H6PD | NM_001282587 | chr1 | 9299902 | 9331394 | GDH/6PGL |
| | | | | | endoplasmic |
| | | | | | bifunctional protein |
| | | | | | isoform 1 precursor |
| HHAT | NM_001122834 | chr1 | 210501595 | 210849638 | protein-cysteine N- |
| | | | | | palmitoyltransferase |
| | | | | | HHAT isoform 1 |
| HS3ST1 | NM_005114 | chr4 | 11399987 | 11430537 | heparan sulfate |
| | | | | | glucosamine 3-O- |
| | | | | | sulfotransferase 1 |
| | | | | | precursor |
| HTR4 | NM_199453 | chr5 | 147830594 | 148016624 | 5-hydroxytryptamine |
| | | | | | receptor 4 isoform g |
| HYAL3 | NM_003549 | chr3 | 50330258 | 50336899 | hyaluronidase-3 isoform |
| | | | | | 1 precursor |
| IL15 | NR_037840 | chr4 | 142557748 | 142655140 | |
| IL5RA | NM_175726 | chr3 | 3108007 | 3152058 | interleukin-5 receptor |
| | | | | | subunit alpha isoform 1 |
| | | | | | precursor |
| KCNAB1 | NM_172159 | chr3 | 156008775 | 156256927 | voltage-gated potassium |
| | | | | | channel subunit beta-1 |
| | | | | | isoform 3 |
| LAMC3 | NM_006059 | chr9 | 133884503 | 133968446 | laminin subunit gamma- |
| | | | | | 3 precursor |
| LDB2 | NM_001290 | chr4 | 16503164 | 16900424 | LIM domain-binding |
| | | | | | protein 2 isoform a |
| LEF1 | NM_001130714 | chr4 | 108968700 | 109090112 | lymphoid enhancer- |
| | | | | | binding factor 1 isoform 3 |
| LPHN3 | NM_015236 | chr4 | 62362838 | 62938168 | latrophilin-3 precursor |
| LRCH1 | NM_015116 | chr13 | 47127295 | 47319036 | leucine-rich repeat and |
| | | | | | calponin homology |
| | | | | | domain-containing |
| | | | | | protein 1 isoform 2 |
| LRP1B | NM_018557 | chr2 | 140988995 | 142889270 | low-density lipoprotein |
| | | | | | receptor-related protein |
| | | | | | 1B precursor |
| LYST | NM_001301365 | chr1 | 235824330 | 236047008 | lysosomal-trafficking |
| | | | | | regulator |
| MAN1A1 | NM_005907 | chr6 | 119498365 | 119670931 | mannosyl- |
| | | | | | oligosaccharide 1,2- |
| | | | | | alpha-mannosidase IA |
| MCTP1 | NM_001002796 | chr5 | 94041241 | 94417570 | multiple C2 and |
| | | | | | transmembrane domain- |
| | | | | | containing protein 1 |
| | | | | | isoform S |
| MFAP3L | NM_021647 | chr4 | 170907747 | 170947581 | microfibrillar-associated |
| | | | | | protein 3-like isoform 1 |
| | | | | | precursor |
| MORC3 | NM_015358 | chr21 | 37692486 | 37748944 | MORC family CW-type |
| | | | | | zinc finger protein 3 |
| MTA1 | NM_001203258 | chr14 | 105886185 | 105937057 | metastasis-associated |
| | | | | | protein MTA1 isoform |
| | | | | | MTA1s |
| NECAP2 | NM_001145278 | chr1 | 16767166 | 16786584 | adaptin ear-binding |
| | | | | | coat-associated protein 2 |
| | | | | | isoform 3 |
| NEIL3 | NM_018248 | chr4 | 178230990 | 178284092 | endonuclease 8-like 3 |
| NLGN4X | NM_181332 | chrX | 5808066 | 6146923 | neuroligin-4, X-linked |
| NMD3 | NM_015938 | chr3 | 160939098 | 160969795 | 60S ribosomal export |
| | | | | | protein NMD3 |
| NOTCH2 | NM_024408 | chr1 | 120454175 | 120612317 | neurogenic locus notch |
| | | | | | homolog protein 2 |
| | | | | | isoform 1 preproprotein |
| NRP2 | NM_018534 | chr2 | 206547223 | 206641880 | neuropilin-2 isoform 4 |
| | | | | | precursor |
| NRXN1 | NM_004801 | chr2 | 50145642 | 51259674 | neurexin-1-beta isoform |
| | | | | | alpha1 precursor |
| NT5C2 | NM_001134373 | chr10 | 104847773 | 104953063 | cytosolic purine 5′- |
| | | | | | nucleotidase |
| NTNG1 | NM_014917 | chr1 | 107682744 | 108024475 | netrin-G1 isoform 3 |
| | | | | | precursor |
| NUP133 | NM_018230 | chr1 | 229577043 | 229644088 | nuclear pore complex |
| | | | | | protein Nup133 |
| PARN | NM_001134477 | chr16 | 14529556 | 14724128 | poly(A)-specific |
| | | | | | ribonuclease PARN |
| | | | | | isoform 2 |
| PCDH7 | NM_032456 | chr4 | 30722029 | 30726957 | protocadherin-7 isoform |
| | | | | | b precursor |
| PCOLCE2 | NM_013363 | chr3 | 142536701 | 142608045 | procollagen C- |
| | | | | | endopeptidase enhancer |
| | | | | | 2 precursor |
| PDE2A | NM_001146209 | chr11 | 72287183 | 72380108 | cGMP-dependent 3′,5′- |
| | | | | | cyclic |
| | | | | | phosphodiesterase |
| | | | | | isoform PDE2A4 |
| PDE6C | NM_006204 | chr10 | 95372344 | 95425429 | cone cGMP-specific |
| | | | | | 3′,5′-cyclic |
| | | | | | phosphodiesterase |
| | | | | | subunit alpha′ |
| PDIA3 | NM_005313 | chr15 | 44038589 | 44064804 | protein disulfide- |
| | | | | | isomerase A3 precursor |
| PDZK1 | NM_001201325 | chr1 | 145727665 | 145764206 | Na(+)/H(+) exchange |
| | | | | | regulatory cofactor |
| | | | | | NHE-RF3 isoform 1 |
| PHTF1 | NM_006608 | chr1 | 114239823 | 114301777 | putative homeodomain |
| | | | | | transcription factor 1 |
| PLEKHA2 | NM_021623 | chr8 | 38758752 | 38831430 | pleckstrin homology |
| | | | | | domain-containing |
| | | | | | family A member 2 |
| POU2F1 | NM_001198783 | chr1 | 167298280 | 167396582 | POU domain, class 2, |
| | | | | | transcription factor 1 |
| | | | | | isoform 2 |
| PRDM16 | NM_022114 | chr1 | 2985741 | 3355185 | PR domain zinc finger |
| | | | | | protein 16 isoform 1 |
| PRDM5 | NM_001300824 | chr4 | 121613067 | 121844021 | PR domain zinc finger |
| | | | | | protein 5 isfoorm 3 |
| PRKCE | NM_005400 | chr2 | 45879042 | 46415129 | protein kinase C epsilon |
| | | | | | type |
| PRKCZ | NM_001033582 | chr1 | 2036154 | 2116834 | protein kinase C zeta |
| | | | | | type isoform 2 |
| PRUNE | NM_021222 | chr1 | 150980972 | 151008189 | protein prune homolog |
| | | | | | isoform 1 |
| PTGS2 | NM_000963 | chr1 | 186640943 | 186649559 | prostaglandin G/H |
| | | | | | synthase 2 precursor |
| PTPRF | NM_130440 | chr1 | 43996546 | 44089343 | receptor-type tyrosine- |
| | | | | | protein phosphatase F |
| | | | | | isoform 2 precursor |
| PTPRZ1 | NM_002851 | chr7 | 121513158 | 121702090 | receptor-type tyrosine- |
| | | | | | protein phosphatase zeta |
| | | | | | isoform 1 precursor |
| PUM1 | NM_014676 | chr1 | 31404352 | 31538564 | pumilio homolog 1 |
| | | | | | isoform 2 |
| RAD52 | NM_001297419 | chr12 | 1020901 | 1099207 | DNA repair protein |
| | | | | | RAD52 homolog |
| | | | | | isoform a |
| RAI2 | NM_001172743 | chrX | 17818168 | 17879457 | retinoic acid-induced |
| | | | | | protein 2 isoform 1 |
| RNF144A | NM_014746 | chr2 | 7057522 | 7184309 | E3 ubiquitin-protein |
| | | | | | ligase RNF144A |
| SCHIP1 | NM_014575 | chr3 | 158991035 | 159615155 | schwannomin- |
| | | | | | interacting protein 1 |
| | | | | | isoform 1 |
| SERTAD2 | NM_014755 | chr2 | 64858754 | 64881046 | SERTA domain- |
| | | | | | containing protein 2 |
| SLC12A6 | NM_001042495 | chr15 | 34522196 | 34630265 | solute carrier family 12 |
| | | | | | member 6 isoform c |
| SLC15A2 | NM_001145998 | chr3 | 121613170 | 121663034 | solute carrier family 15 |
| | | | | | member 2 isoform b |
| SLC4A4 | NM_003759 | chr4 | 72204769 | 72437804 | electrogenic sodium |
| | | | | | bicarbonate |
| | | | | | cotransporter 1 isoform 2 |
| SMYD3 | NM_022743 | chr1 | 245912641 | 246580714 | histone-lysine N- |
| | | | | | methyltransferase |
| | | | | | SMYD3 isoform 2 |
| SNTG2 | NM_018968 | chr2 | 946553 | 1371384 | gamma-2-syntrophin |
| SPATS2L | NM_001100424 | chr2 | 201170984 | 201346986 | SPATS2-like protein |
| | | | | | isoform b |
| TBL1X | NM_001139468 | chrX | 9431334 | 9687780 | F-box-like/WD repeat- |
| | | | | | containing protein |
| | | | | | TBL1X isoform b |
| TGFBR3 | NM_001195683 | chr1 | 92145899 | 92351836 | transforming growth |
| | | | | | factor beta receptor type |
| | | | | | 3 isoform b precursor |
| THRAP3 | NM_005119 | chr1 | 36690016 | 36770957 | thyroid hormone |
| | | | | | receptor-associated |
| | | | | | protein 3 |
| TIAM1 | NM_003253 | chr21 | 32490735 | 32931290 | T-lymphoma invasion |
| | | | | | and metastasis-inducing |
| | | | | | protein 1 |
| TLE4 | NM_007005 | chr9 | 82186687 | 82341796 | transducin-like enhancer |
| | | | | | protein 4 isoform 3 |
| TNIK | NM_001161561 | chr3 | 170780291 | 171178197 | TRAF2 and NCK- |
| | | | | | interacting protein |
| | | | | | kinase isoform 3 |
| TRIM48 | NM_024114 | chr11 | 55029657 | 55038595 | tripartite motif- |
| | | | | | containing protein 48 |
| TRPM8 | NM_024080 | chr2 | 234826042 | 234928166 | transient receptor |
| | | | | | potential cation channel |
| | | | | | subfamily M member 8 |
| TSPAN9 | NM_001168320 | chr12 | 3186520 | 3395730 | tetraspanin-9 |
| TTF1 | NM_001205296 | chr9 | 135250936 | 135282238 | transcription termination |
| | | | | | factor 1 isoform 2 |
| VPS8 | NM_015303 | chr3 | 184529930 | 184770402 | vacuolar protein sorting- |
| | | | | | associated protein 8 |
| | | | | | homolog isoform b |
| WASF3 | NM_001291965 | chr13 | 27131839 | 27263082 | wiskott-Aldrich |
| | | | | | syndrome protein family |
| | | | | | member 3 isoform 2 |
| WBSCR16 | NM_001281441 | chr7 | 74470621 | 74489717 | Williams-Beuren |
| | | | | | syndrome chromosomal |
| | | | | | region 16 protein |
| | | | | | isoform 3 |
| WDFY3 | NM_014991 | chr4 | 85590692 | 85887544 | WD repeat and FYVE |
| | | | | | domain-containing |
| | | | | | protein 3 |
| WISP1 | NM_080838 | chr8 | 134203281 | 134243932 | WNT1-inducible- |
| | | | | | signaling pathway |
| | | | | | protein 1 isoform 2 |
| | | | | | precursor |
| XRCC5 | NM_021141 | chr2 | 216974019 | 217071016 | X-ray repair cross- |
| | | | | | complementing protein 5 |
| YEATS2 | NM_018023 | chr3 | 183415605 | 183530413 | YEATS domain- |
| | | | | | containing protein 2 |
| ZNF274 | NM_133502 | chr19 | 58694355 | 58724928 | neurotrophin receptor- |
| | | | | | interacting factor |
| | | | | | homolog isoform c |
| ZNF702P | NR_003578 | chr19 | 53471503 | 53496784 | |

### REFERENCES

Abecasis G R, Altshuler D, Auton A, Brooks L D, Durbin R M, et al. (2010) A map of human genome variation from population-scale sequencing. Nature 467: 1061-1073.
Assmann G, Schulte H (1988) The prospective cardiovascular münster (procam) study: prevalence of hyperlipidemia in persons with hypertension and/or diabetes mellitus and the relationship to coronary heart disease. American heart journal 116: 1713-24.
Bell D, Berchuck A, Birrer M, Chien J, Cramer D, et al. (2011) Integrated genomic analyses of ovarian carcinoma. Nature 474: 609-15.
Benjamin E J, Wolf P A, D'Agostino R B, Silbershatz H, Kannel W B, et al. (1998) Impact of atrial fibrillation on the risk of death: the framingham heart study. Circulation 98: 946-52.
Church D M, Schneider V A, Graves T, Auger K, Cunningham F, et al. (2011) Modernizing reference genome assemblies. PLoS biology 9: 1001091.
Mills R E, Walter K, Stewart C, Handsaker R E, Chen K, et al. (2011) Mapping copy number variation by population-scale genome sequencing. Nature 470: 59-65.
Motakis E, Ivshina A V, Kuznetsov V A (2009) Data-driven approach to predict survival of cancer patients: estimation of microarray genes' prediction significance by cox proportional hazard regression model. IEEE Eng Med Biol Mag 28: 58-66.
Tothill R W, Tinker A V, George J, Brown R, Fox S B, et al. (2008) Novel molecular subtypes of serous and endometrioid ovarian cancer linked to clinical outcome. Clin Cancer Res 14: 5198-5208.

1. An in vitro method for obtaining information on the number of DNA copies (CN) of a given locus of interest in a biological sample, the method comprising:
i) obtaining the CN value of the locus of interest in the biological sample;
ii) obtaining the CN value or values of one or more CN-invariant locus reference(s) (CNILR) in the biological sample, wherein the CNILR is defined as a which is locally CN-invariant, or as a locus with a minimal coefficient of variation value of its CN values across said group;
iii) obtaining the CN value or values of or one or more CN-invariant survival-insignificant locus reference(s) (CNISILR), wherein the CNISILR being defined as a CNILR, whose CN value, or any expression value of the genes within the locus, cannot define more than one subgroup of said group, based on survival prediction analysis; and
iv) normalizing the CN value of the locus of interest by the CN value of said one or more CNISILRs if defined, otherwise normalizing the CN value of the locus of interest by the CN value of said one or more CNILRs.

2. The method according to claim 1, wherein said one or more CNILRs in the biological sample is/are determined by:
i) providing a representative reference data set containing measurements of genome wide CN variation with respect to a group of samples;
ii) identifying a set of loci with the lowest variation across the reference data set as the reference loci;
iii) ranking the reference loci by their median CN values across the reference data set; and
iv) selecting one locus or a set of loci with the highest median CN value(s) as the CNILR(s).

3. The method according to claim 1, wherein said one or more CNISILRs in the biological sample is/are determined by:
i) providing a representative reference data set containing measurements of genome-wide CN variation with respect to a group of samples;
ii) identifying a set of loci with the lowest variation across the reference data set as the reference loci;
iii) identifying a subset of loci, whose functions and/or transcriptional activity are not statistically associated in the reference data set, as loci with no significant statistical association;
iv) ranking the loci with no significant statistical association by the coefficients of variation of the expression values of the transcripts originating in these loci across the reference data set; and
v) selecting one locus or a set of loci with the lowest coefficient(s) of variation of the CN values as the CNISILRs.

4. The method according to claim 1, wherein normalization is conducted by normalizing the CN value of the locus of interest by the CN value of the CNISILs determined by:
i) providing a representative reference data set containing measurements of genome-wide CN variation with respect to a group of samples;
ii) identifying a set of loci with the lowest variation across the reference data set as the reference loci;
iii) identifying a subset of loci, whose functions and/or transcriptional activity are not statistically associated in the reference data set, as loci with no significant statistical association;
iv) ranking the loci with no significant statistical association by the coefficients of variation of the expression values of the transcripts originating in these loci across the reference data set; and
v) selecting one locus or a set of loci with the lowest coefficient(s) of variation of the CN values as the CNISILRs.

5. The method according to claim 1, wherein normalization is conducted by normalizing the CN values of the locus of interest by the median CN values of more than one CNISILRs determined by:
i) providing a representative reference data set containing measurements of genome-wide CN variation with respect to a group of samples;
ii) identifying a set of loci with the lowest variation across the reference data set as the reference loci;
iii) identifying a subset of lad, whose functions and/or transcriptional activity are not statistically associated in the reference data set, as loci with no significant statistical association;
iv) ranking the loci with no significant statistical association by the coefficients of variation of the expression values of the transcripts originating in these loci across the reference data set; and
v) selecting one locus or a set of loci with the lowest coefficient(s) of variation of the CN values as the CNISILRs.

6. The method according to claim 1, wherein normalization is conducted by normalizing the CN value of the locus of interest by the CN value of one CNILR determined by:
i) providing a representative reference data set containing measurements of genome-wide CN variation with respect to a group of samples;
ii) identifying a set of loci with the lowest variation across the reference data set as the reference loci;
iii) ranking the reference loci by their median CN values across the reference data set; and
iv) selecting one locus or a set of loci with the highest median CN value(s) as the CNILR(s).

7. The method according to claim 1 wherein normalization is conducted by normalizing the CN values of the locus of interest by the median CNILRs determined by:
i) providing a representative reference data set containing measurements of genome-wide CN variation with respect to a group of samples;
ii) identifying a set of loci with the lowest variation across the reference data set as the reference loci;
iii) ranking the reference loci by their median CN values across the reference data set; and
iv) selecting one locus or a set of loci with the highest median CN value(s) as the CNILR(s).

8. The method according to claim 1, wherein said one or more CNILRs or CNISILRs is one or more loci from the group consisting of:
XRCC5; AUTS2; EIF5; PARN; YEATS2; and FHL2.

9. The method according to claim 1, wherein said one or more CNILRs or CNISILRs is/are selected from the loci identified in Table 1, Table 2, Table 3, Table 4, Table 5, Table 8, Table 9, Table 10, Table 11, Table 13 or Table 14.

10. The method according to claim 1, wherein the method for obtaining the CN value of the locus of interest and/or of said reference locus or loci in the biological sample is a qPCR-based assay or qCGH/tiling array-based assay.

11. The method according to claim 1, wherein the CN value of the locus of interest and/or of said reference locus or loci in the biological sample is determined as a gene expression value originating from a transcript of said locus.

12. The method according to claim 1, wherein the sample is obtained from cells or tissues from cancer patients or cell cultures derived from cancer patients.

13. The method according to claim 12, wherein the cancer type or subtype is selected from ovarian cancer, breast invasive carcinomas, head and neck squamous cell carcinoma, lung adenocarcinoma, lung squamous cell carcinoma, prostate adenocarcinoma, colon adenocarcinoma, stomach adenocarcinoma, hepatocellular carcinoma, or cervical squamous cell carcinoma.

14. The method according to claim 1, wherein the loci are cytobands.

15. The method according to claim 1, wherein said one or more CNILRs or CNISILRs is/are selected if the coefficient of variation is less than a computationally or empirically predetermined threshold equal to 0.05.

16. The method according to claim 1 wherein the sample is obtained from cells or tissues obtained from myocardial infarction patients or cell cultures derived from myocardial infarction patients.

17. A kit for use in an in vitro method for obtaining information on the number of DNA copies (CN) of a given locus of interest in a biological sample, the method comprising:
i) obtaining the CN value of the locus of interest in the biological sample;
ii) obtaining the CN value or values of one or more CN-invariant locus reference(s) (CNILR) in the biological sample, wherein the CNILR is defined as a which is locally CN-invariant, or as a locus with a minimal coefficient of variation value of its CN values across said group;
iii) obtaining the CN value or values of or one or more CN-invariant survival-insignificant locus reference(s) (CNISILR), wherein the CNISILR being defined as a CNILR, whose CN value, or any expression value of the genes within the locus, cannot define more than one subgroup of said group, based on survival prediction analysis; and
iv) normalizing the CN value of the locus of interest by the CN value of said one or more CNISILRs if defined, otherwise normalizing the CN value of the locus of interest by the CN value of said one or more CNILRs, wherein the kit comprises:
A) oligonucleotide primers capable of binding to and/or amplifying at least a portion of the nucleic add sequence, and/or cDNA derived therefrom, of at least one locus selected from the group consisting of: XRCC5; AUTS2; EIF5; PARN; YEATS2; and FHL2; or
B) oligonucleotide primers capable of binding to and/or amplifying at least a portion of the nucleic add sequence, and/or cDNA derived therefrom, of at least one locus selected from Table 1, Table 2, Table 3, Table 4, Table 5, Table 8, Table 9, Table 10, Table 11, Table 13, or Table 14.

18. The kit according to claim 17, wherein
A) the primer sequences are selected from or derived from oligonucleotide sequences identified in Table 6 as SEQ ID Nos: 1-24.

19. (canceled)

20. A computer program or a computer device comprising a computer program which is capable of implementing the method comprising:
i) obtaining the CN value of the locus of interest in the biological sample;
ii) obtaining the CN value or values of one or more CN-invariant locus reference(s) (CNILR) in the biological sample, wherein the CNILR is defined as a which is locally CN-invariant, or as a locus with a minimal coefficient of variation value of its CN values across said group;
iii) obtaining the CN value or values of or one or more CN-invariant survival-insignificant locus reference(s) (CNISILR), wherein the CNISILR being defined as a CNILR, whose CN value, or any expression value of the genes within the locus, cannot define more than one subgroup of said group, based on survival prediction analysis; and
iv) normalizing the CN value of the locus of interest by the CN value of said one or more CNISILRs if defined, otherwise normalizing the CN value of the locus of interest by the CN value of said one or more CNILRs.


SEQUENCE LISTING (24 sequences)

SEQ ID NO: 1 - DNA, 20 bp, Homo sapiens
aggtcgtggatgtatgggga

SEQ ID NO: 2 - DNA, 20 bp, Homo sapiens
ggccgcatccaacttgtttt

SEQ ID NO: 3 - DNA, 20 bp, Homo sapiens
gtaaggtgcacgtttcctga

SEQ ID NO: 4 - DNA, 20 bp, Homo sapiens
ctctaactcgcgatggctcc

SEQ ID NO: 5 - DNA, 20 bp, Homo sapiens
accgagaactcttgcagtcg

SEQ ID NO: 6 - DNA, 20 bp, Homo sapiens
agaactggtctgacacgctg

SEQ ID NO: 7 - DNA, 20 bp, Homo sapiens
cccaccatagctgcctgaaa

SEQ ID NO: 8 - DNA, 20 bp, Homo sapiens
catacggcaagccctctcat

SEQ ID NO: 9 - DNA, 20 bp, Homo sapiens
cccgagtgcccatcatcatt

SEQ ID NO: 10 - DNA, 20 bp, Homo sapiens
ccttctgtacttgcagccct

SEQ ID NO: 11 - DNA, 20 bp, Homo sapiens
gaagtgctccctctcactgg

SEQ ID NO: 12 - DNA, 20 bp, Homo sapiens
gcaagattgcctgggtgaga

SEQ ID NO: 13 - DNA, 20 bp, Homo sapiens
accaagtggagacacagcag

SEQ ID NO: 14 - DNA, 20 bp, Homo sapiens
tccccatacatccacgacct

SEQ ID NO: 15 - DNA, 20 bp, Homo sapiens
tgtaaggtgcacgtttcctg

SEQ ID NO: 16 - DNA, 20 bp, Homo sapiens
aggttgacctgttacggctg

SEQ ID NO: 17 - DNA, 20 bp, Homo sapiens
ctgtcaatgtcaaccgcagc

SEQ ID NO: 18 - DNA, 20 bp, Homo sapiens
gcctttgcaacgtcaaccat

SEQ ID NO: 19 - DNA, 20 bp, Homo sapiens
gtggcgctgtgttcactttc

SEQ ID NO: 20 - DNA, 20 bp, Homo sapiens
aatgggctgggacatgttgt

SEQ ID NO: 21 - DNA, 20 bp, Homo sapiens
aggaatgacggggactccat

SEQ ID NO: 22 - DNA, 20 bp, Homo sapiens
aatgatgatgggcactcggg

SEQ ID NO: 23 - DNA, 20 bp, Homo sapiens
tcgagtaaggcacacccaaa

SEQ ID NO: 24 - DNA, 20 bp, Homo sapiens
tagacttgacgcaacgggag